SEQUENCE LISTING (26 sequences)

SEQ ID NO: 1 - DNA, 442 bp, Solanum lycopersicum
gtaaaacgacggccagtggcactcaactttataaaccctgacgaactttcaatgcagtgcattttgatcgcattgaacaggtttcttcaggagaaacatggttcaaaaatggcctttttagatggtaatcctcctgagagactttgcatgccgattgttgaacacattgagtcaaaaggtggccaagtcagactgaactcacgaataaaaaagattgagctgaatgaggatggaagtgtcaagagttttatactgagtgacggtagtgcaatcgagggagatgcttttgtgtttgccgctccagtggatattttcaagcttctattgcctgaagactggaaagagattccatatttccaaaagttggagaagttagtcggagtacctgtgataaatgtacatatatggtttgacagaaaactgaagcatggtcatagctgtt

SEQ ID NO: 2 - DNA, 299 bp, Citrus Sinensis
acccatcatccaaaattcaatctttaacaacaaattctcttcagaaaagcctcttcatatttcatcaactcaaaatctcacattttcgccaaaagagcaacaaaaagagcttaaaacacagtgcaatgcctatgaggctgacaggtcacgtcctctggacataaacatcgaggttcttgatgagcaggctcggtttgaggctgcccagaggctcaaaattggcatatatttcgccacatggtgggctttgaatgtggtgtttaatatctataacaaaaaagtgttgaatgcttttcctt

SEQ ID NO: 3 - DNA, 357 bp, Artificial Sequence
  random dsDNA sequence
gtaaaacgacggccagtgatgagtctgggtggagcgcgccccatttataccgtgagtagggtcgaccaagaaccgcaagatgcgtcggtgtacaaataattgtcaacagaccgtcgtgttttgaaaatggtaccagcatcttcgggcggtctcaatcaagcatggattacggttgaactaatacgtatactttgcacgggttcactgcggtccgttcagagtcgaccaaggacacaatcgagctcccatctgtatgctcgactaacttgtacccaacccccggagcttggcagctcctggggtatcatggagcctctggttcatcccgtgggatatcaagccatggtcatagctgtt

SEQ ID NO: 4 - DNA, 533 bp, Solanum lycopersicum
gtaaaacgacggccagtgtgaaatcgacggttcatttggggagagtgagcactggtggctttaacaatggagagaaggagatttttggggagaagatcagagggagtttgaacaacaatctcaggattaatcagttgtcgaaaagtttgaaacttgagaagaagattaaacctggggttgcttactctgtgatcactactgaaaatgacacagagactgtgttcgtagatatgccacgtcttgagagacgccgggcaaatcccaaggatgtggctgcagtcatattaggaggaggcgaagggaccaagttattcccacttacaagtagaactgcaacccctgctgttccggttggaggatgctacaggctgatagacatcccaatgagcaactgtatcaacagtgctattaacaagatttttgtgctgacacagtacaattctgctgccctgaatcgtcacattgctcgaacgtattttggcaatggtgtgagctttggagatggatttgtcgaggtcatggtcatagctgtt

SEQ ID NO: 5 - DNA, 371 bp, Solanum lycopersicum
aacgtgaagaatcaaagggagaacttggttctccagttggctaactctcaaatgcgccttcaacctccgtctaccgccccggaccgccttcactatggtgttctccggcagttccggcagaagctattgaagaattactcatcttggtgttcatatctggggaagaagtcacaggttcgactgccgagacgtcaaaatccagaaatttctcgtcgtgaactcttgtatgtatgtctgtatttattgatttggggtgaggctgcaaatctccgttttgttcctgaatgtttgtgttatatctatcatcatatggctatggaactaaattacattcttgatggtcacattgatgagaatactggacacccttt

SEQ ID NO: 6 - DNA, 400 bp, Solanum lycopersicum
ttgcttcaagaattccttaagaagcatggtggtgtcagatatccaacaatacttggagttagagagcatatattcactggcagtgtttcatctcttgcatggtttatgtcgaatcaggagactagttttgtaactatagggcagagattgttggctcaccctttgaaagtccggttccactatggtcatccagatgtgtttgatagattgtttcaccttacaagagggggtgtcagcaaagcttccaaagttattaacttaagtgaggacatatttgcaggtttcaattctacactacgtgaaggcaacgtcactcatcatgaatatatacaagttggtaagggaagagatgtaggtcttaatcaaatatctctgtttgaggcaaagatagctaatggta

SEQ ID NO: 7 - DNA, 299 bp, Citrus sinensis
acattcgttcaggaatcaccattataatggagaaagcaacaattgaagatggaatggtcatatagatgagtctcatacctttcatgaactggtttaaaatggggattaatcaagcattggaaagccgtaatgtccgtggctgatgaggaagctatgaagatcacaagagcagcattttggcctggaaagaagcaagctgcgtgtttataccacatgtatattagaagctcctatcctatatgtataaaacgagcctaggacaacaatatatgcgagtagttttcaaataaaatcagtag

SEQ ID NO: 8 - DNA, 634 bp, Solanum lycopersicum
gtaaaacgacggccagtgatggccttctctgctggacaatcttatgcattttcaacaaattttgatcccttgcaacaaaatttatggggttgtaagcctcatatttcatcattttccctcaaagatatcaactttaaacaatgtgataagcctaacatcttgtctaaaaaacctctatacatttcagcagtactaagtggattcggacatgctgatgaatcaaaagagttcaaatctagggaccctttagtccaatgcaatgcatatgaagctagtcgaccccaatcgataccaatcaacattgagtttggccaagaagctcaggctgctgctactcagaagctcaagattggactctattttgcaacatggtgggctttgaatgttgttttcaatatttacaacaagaaggttttgaatgcatttccatttccatggcttacttctactctttctctggctgctggctctcttatgatgttggtttcttgggctactaagattgctgaaactccaaagactgactttgatttttggaaagccttgtttcctgttgctgtggctcacacaattggccatgtggctgcaacagtgagcatgtcaaaagttgctgtttcacatggtcatagctgtt

SEQ ID NO: 9 - DNA, 274 bp, Citrus sinensis
gtaaaacgacggccagtcgggctctgtttgcagttaccaatgttgccactgaggttaactctgttgagcaggcccagaagagatccaaggaaagccagaggccggtgtatccatttactgcaatagtgggacaggaagagatgaaattgtgccttttgttaaatgtgattgatcccaaaattggtggtgttatgattatgggtgatagaggaacggggaaatccaccactgttaggtccttggtagatttactccccgcatggtcatagctgtt

SEQ ID NO: 10 - DNA, 302 bp, Citrus sinensis
gtaaaacgacggccagtgcaaagaccagctcagctgtggagaagagctatgaactacctgatgggcaggtcatcactattggggctgaacgattcaggtgtccagaagtcctcttccaaccatccatgatcgggatggaagctgctggcattcatgaaacaacatacaactccattatgaagtgtgatgtcgatatcagaaaggatctctatggcaatattgtccttagtggtggctccaccatgttcccaggtattgctgacagaatgagcaaggagatcactgccatggtcatagctgtt

SEQ ID NO: 11 - DNA, 20 bp, Artificial Sequence
  Single strand DNA oligonucleotide
ttgctgaccgtatgagcaag

SEQ ID NO: 12 - DNA, 20 bp, Artificial Sequence
  Single strand DNA oligonucleotide
ggacaatggatggaccagac

SEQ ID NO: 13 - DNA, 20 bp, Artificial Sequence
  Single strand DNA oligonucleotide
gttcttcacgttgaagccaa

SEQ ID NO: 14 - DNA, 20 bp, Artificial Sequence
  Single strand DNA oligonucleotide
gtcctcaagcctggtatggt

SEQ ID NO: 15 - DNA, 17 bp, Artificial Sequence
  Single strand DNA oligonucleotide
ttcgtgtggtgggtagc

SEQ ID NO: 16 - DNA, 17 bp, Artificial Sequence
  Single strand DNA oligonucleotide
gagcattgacgggttga

SEQ ID NO: 17 - DNA, 23 bp, Artificial Sequence
  Single strand DNA oligonucleotide
ggctacgtccaacaagtaactca

SEQ ID NO: 18 - DNA, 20 bp, Artificial Sequence
  Single strand DNA oligonucleotide
ccggtgactacacgaaacag

SEQ ID NO: 19 - DNA, 25 bp, Artificial Sequence
  Single strand DNA oligonucleotide
aaccatattgacagaaatgctgata

SEQ ID NO: 20 - DNA, 20 bp, Artificial Sequence
  Single strand DNA oligonucleotide
cagcccaaaatctgatgctc

SEQ ID NO: 21 - protein, 29 aa, Artificial Sequence
  Synthetic Peptide
KHKHKHKHKHKHKHKHKHKKLFKKILKYL

SEQ ID NO: 22 - protein, 32 aa, Artificial Sequence
  Synthetic Peptide
GLFEAIEGFIENGWEGMIDGWYGRRRRRRRRR

SEQ ID NO: 23 - DNA, 20 bp, Artificial Sequence
  Single strand DNA oligonucleotide
tctcagaagacgaagcgtga

SEQ ID NO: 24 - DNA, 19 bp, Artificial Sequence
  Single strand DNA oligonucleotide
cggagagcctcattgtcct

SEQ ID NO: 25 - DNA, 20 bp, Artificial Sequence
  Single strand DNA oligonucleotide
ttgctgaccgtatgagcaag

SEQ ID NO: 26 - DNA, 20 bp, Artificial Sequence
  Single strand DNA oligonucleotide
ggacaatggatggaccagac

(19) United States
(12) Patent Application Publication
(10) Pub. No.: US 2018/0237790 A1
(43) Pub. Date: Aug. 23, 2018

(54) FORMULATIONS AND COMPOSITIONS FOR DELIVERY OF NUCLEIC ACIDS TO PLANT CELLS

(71) Applicant: Forrest Innovation Ltd., Caesarea (IL)

(72) Inventors: Eyal MAORI, Rishon-LeZion (IL); Alon WELLNER, Herzlia (IL); Avital WEISS, Karkur (IL); Roy BOROCHOV, Kfar-Bin-Nun (IL); Jonathan HENEN, Moshav Beit Yitzhak (IL)

(21) Appl. No.: 15/752,274

(22) PCT Filed: Aug. 11, 2016
(86) PCT No.: PCT/IL2016/050877

§ 371 Date: Feb. 13, 2018

Related U.S. Application Data
(60) Provisional application No. 62/204,603, filed on Aug. 13, 2015.

(51) Int. Cl. C12N 15/82

(52) U.S. Cl. CPC C12N 15/8206; C12N 15/8218

(57) ABSTRACT

The present invention, in some embodiments thereof, relates to methods and compositions for delivering polynucleotides into plant cells having a cell wall, and, more particularly, but not exclusively, to methods of delivering dsRNA into plant cells and plants. In particular, the present invention provides compositions and methods for delivering the polynucleotides through the cell wall and enhancing fitness, vigor, biotic and abiotic stress tolerance.

### FIELD AND BACKGROUND OF THE INVENTION

The present invention, in some embodiments thereof, relates to gene silencing in plant cells and plants, and, more particularly, but not exclusively, to compositions and methods for efficient delivery of nucleic acids active in RNA pathways to plant cells and plants.

The process of post-transcriptional gene silencing, an evolutionarily conserved cellular defense mechanism preventing expression of foreign genes in diverse flora and phyla, has been the focus of intense interest since first described by Fire (Trends Genet 1999, 15:358-363). Application of RNAi-based gene silencing technology in plants holds out promise of affecting both endogenous plant traits and, via transfer of dsRNA and cleavage products siRNA and miRNA, gene expression in other, plant-associated (e.g. pathogenic or symbiotic) organisms, including viruses, bacteria, fungi, nematodes, insects, other plant species, and animals (for review see Saurabh et al, Planta 2014).

The lipophilic and anionic nature of cell membranes poses serious challenges for the delivery of negatively charged molecules, such as polynucleotides and even oligonucleotides, into the cells due to their size and charge. Various approaches to deliver negatively-charged biomolecules into cells include viral-based delivery systems and non-viral based delivery systems such as liposomes, polymers, calcium phosphate, electroporation, and micro-injection techniques. In planta methods for delivery include meristem transformation, floral dip and pollen transformation.

Methods for effective delivery of dsRNA to target plants, however, must contend with the unique morphology of the plant, including the waxy cuticle, hardened cortex or bark, and the rigid plant cell wall, and finally, the plant cell membrane. To date, plant recombinant techniques have relied mostly on indirect methods, such as plant viral or pathogenic agrobacterial species (e.g. A. tumefaciens) for efficient transfer of nucleic acids into plant cells, however, significant technical and regulatory hurdles prevent widespread commercial use of such techniques. Thus, methods for direct application in plants, suitable for effective transfer of active dsRNA to plant cells, are in great demand. Despite this interest, however, commercially viable practical solutions for dsRNA delivery to plants are still not available.

U.S. Patent Application Publication No. 2011005836 to Eudes and Chugh describes the use of a carrier moiety which can be loaded with a charged biomolecule (e.g. polynucleotide) and which can traverse plant cell membrane and/or cell wall. Their preferred carrier moiety is a cell penetrating peptide, but effective results still required prior permeabilization of the cells. Jain et al (FEBS 2014) describes the use of such a carrier moiety comprising the antimicrobial peptide tachyplesin as a non-viral macromolecular carrier for plant cell transformation.

Another vehicle (“geodate”) for delivery of a charged (e.g. polynucleotide) cargo across cell membranes, including plant cells, is described by Mannino et al (US 20130224284), comprising lipid and hydrophobic layers.

Peterson et al (US20110203013) provided a delivery vehicle comprising a nanoparticle and microparticle in a lipid compound, for delivery of a biomolecule, including nucleic acids into plant cells by particle bombardment.

Tang et al. (Plant Sci 2006 and U.S. Patent Application Publication No. 20130047298) proposed the use of laser induced stress waves (see US20100216199 to Obara et al and also PCT Publication WO 2009/140701 to Zeiler et al) for dsRNA delivery to plant cells, but demonstrated successful transformation in plant cell culture only.

Sammons et al (U.S. Patent Application Publication No. 20140057789) have described the use of carborundum and/or surfactants to facilitate transfer of polynucleotides to plant cells in planta via direct, topical application.

Other relevant publications include U.S. Pat. Nos. 8,686,222 and 8,664,375.

### SUMMARY OF THE INVENTION

According to an aspect of some embodiments of the present invention, there is provided a method of delivering a polynucleotide to a plant cell comprising contacting the plant cell with the polynucleotide and at least one cell wall degrading enzyme, and at least one of a nucleic acid condensing agent, a transfection reagent, a surfactant, and a cuticle penetrating agent.

According to an aspect of some embodiments of the present invention, there is provided a method of expressing a nucleic acid sequence in a plant cell, the method comprising delivering a polynucleotide to cells of the plant according to the method of the invention, wherein the polynucleotide comprises a nucleic acid construct comprising the nucleic acid sequence transcriptionally connected to a plant expressible promoter.

According to an aspect of some embodiments of the present invention, there is provided a method of increasing vigor, yield and/or tolerance of a plant to biotic and abiotic stress, the method comprising:

delivering a polynucleotide to cells of the plant according to the method of the invention, wherein expression of the polynucleotide in the plant increases vigor, yield and/or tolerance of a plant to biotic and abiotic stress of the plant.

According to an aspect of some embodiments of the present invention, there is provided a method of delivering an agrochemical molecule to a host organism comprising: delivering the agrochemical molecule to a plant comprising:

(a) contacting the plant cell with the agrochemical molecule and a cell wall degrading enzyme and at least one of a nucleic acid condensing agent, a transfection reagent, a surfactant, and a cuticle penetrating agent, thereby delivering the agrochemical molecule to the plant, and

(b) contacting the host organism with the plant,

wherein the host organism ingests cells, tissue or cell contents of the plant.

According to an aspect of some embodiments of the present invention, there is provided a composition of matter comprising a polynucleotide, a cell wall degrading enzyme and at least one of a nucleic acid condensing agent, a transfection reagent, a surfactant, and a cuticle penetrating agent.

According to some embodiments of the present invention, the polynucleotide is an RNA or DNA.

According to some embodiments of the present invention, the polynucleotide is a dsRNA.

According to some embodiments of the present invention, the dsRNA is selected from the group consisting of siRNA, shRNA and miRNA.

According to some embodiments of the present invention, the dsRNA comprises a nucleotide sequence complementary to a sequence of an mRNA selected from the group consisting of Citrus sinensis magnesium-chelatase subunit ChlI, chloroplastic mRNA (SEQ ID NO: 9) Tomato GPT (tomato Glucose phosphate transporter mRNA (SEQ ID NO: 8), Citrus AGPase (citrus glucose-1-phosphate adenylyltransferase large subunit) mRNA (SEQ ID NO: 7) and Citrus CalS Solanum lycopersicum callose synthase mRNA (SEQ ID NO: 6).

According to some embodiments of the present invention, the cell wall degrading enzyme is selected from the group consisting of cellulases, hemicellulases, lignin-modifying enzymes, cinnamoyl ester hydrolases and pectin-degrading enzymes.

According to some embodiments of the present invention, the at least one cell wall degrading enzyme comprises a combination of cellulases, xylases and laminarinases.

According to some embodiments of the present invention, the nucleic acid condensing agent is selected from the group consisting of protamine, spermidine3+, spermine4+, hexamine cobalt, polycationic peptides such as polylysine and polyarginine, histones H1 and H5 and polymers such as PEG, polyaspartate and polyglutamate.

According to some embodiments of the present invention, the transfection reagent is selected from the group consisting of cationic and polycationic polymers, particles and nanoparticles, and cationic and polycationic lipids.

According to some embodiments of the present invention, the surfactant is selected from the group consisting of anionic surfactants, cationic surfactants, amphoteric surfactants and non-ionic surfactants.

According to some embodiments of the present invention, the cuticle penetrating agent is selected from the group consisting of an oil, an abrasive, a fatty acid, a wax, a soap and a grease.

According to some embodiments of the present invention, the contacting is effected by a method selected from the group consisting of spraying, dusting, soaking, injecting, aerosol application, particle bombardment, irrigation, positive or negative pressure application, girdling, ground deposition, trunk drilling and shoot drilling.

According to some embodiments of the present invention, the contacting is effected via spraying, dusting, aerosol application or particle bombardment, the method comprising:

contacting a plant or organ thereof comprising the plant cell with the surfactant or cuticle penetrating agent or both, and

subsequently contacting the plant or organ thereof with the polynucleotide and the cell wall degrading enzyme and the at least one of the nucleic acid, the condensing agent, the transfection reagent and the surfactant,

thereby delivering the polynucleotide to the plant cell.

According to some embodiments of the present invention, the contacting is effected via injection, the method comprising injecting a plant or organ thereof comprising the plant cell with the polynucleotide and the cell wall degrading enzyme and at least one of a the nucleic acid condensing agent, the transfection reagent and the surfactant,

thereby delivering the polynucleotide to the plant cell.

According to some embodiments of the present invention, the contacting is effected via irrigation, the method comprising contacting the a plant or organ thereof comprising the plant cell with the polynucleotide and the cell wall degrading enzyme and at least one of a the nucleic acid condensing agent, the transfection reagent and the surfactant, thereby delivering the polynucleotide to the plant cell.

According to some embodiments of the present invention, the plant cell comprises a cell wall.

According to some embodiments of the present invention, the plant organ is selected from the group consisting of a leaf, a stem, a root, a flower, a fruit, a bud, a shoot, a tuber, a bulb, a seed, an embryo and a seed pod.

According to some embodiments of the present invention, the composition is formulated for administration by a method selected from the group consisting of spraying, dusting, soaking, injecting, aerosol application, particle bombardment, irrigation, positive or negative pressure application, girdling, ground deposition, trunk drilling and shoot drilling.

According to some embodiments of the present invention, the composition is formulated for spraying or topical administration, comprising the polynucleotide, the cell wall degrading enzyme and at least one of a nucleic acid condensing agent, a transfection reagent, a surfactant, and a cuticle penetrating agent.

According to some embodiments of the present invention, the composition is formulated for irrigation, comprising the polynucleotide, the cell wall degrading enzyme and at least one of a nucleic acid condensing agent, a transfection reagent, a surfactant, and a cuticle penetrating agent.

According to some embodiments of the present invention, the composition further comprises an agrochemical molecule.

According to some embodiments of the present invention, the agrochemical molecule is selected from the group consisting of fertilizers, pesticides, fungicides and antibiotics.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying Drawings. With specific reference now to the Drawings in detail, it is stressed that the particulars shown are by way of example and for purposes of illustrative discussion of embodiments of the invention. In this regard, the description taken with the Drawings makes apparent to those skilled in the art how embodiments of the invention may be practiced.

In the Drawings:

FIG. 1 is a photo of agarose gel separation of dsRNA-peptide KH₉-BP100 (SEQ ID NO: 21) complex, prepared in a molar ratio of 20-1000 (Peptide:dsRNA). 500 ng dsRNA was mixed with the indicated amounts of peptide, and 1 μl of the complex was separated on agarose gel. The gel was stained with ethidium bromide;

FIG. 2 illustrates the effect of Sodium Phosphate buffer concentration on dsRNA:Peptide complex aggregation in solution. Binocular microphotographs of drops of freshly prepared solutions of dsRNA:Protein in molar ratios of 10, 500 and 2000:1 (Peptide:dsRNA) at 3 or 10 mM NaPO₄ buffer, pH 6.8 were observed for aggregation (white clumps);

FIG. 3 is a photo of agarose gel separation of the dsRNA-peptide complex formed in 3 or 10 mM NaPO₄ buffer, showing the greater complex formation with higher peptide:dsRNA molar ratios;

FIGS. 4A-4E are a series of photos demonstrating toxicity of different concentrations of cell wall degrading enzyme (CWDE) topically applied to Tiny Tim tomato plants. 100 μl of increasing concentrations of CWDE (0.001, 0.01, 0.1 and 1.0 mg/ml) was applied topically to leaves of 18 day old Tiny Tim tomato plants immediately after spraying with carborundum. T=treated leaves, C=untreated, control leaves;

FIGS. 5A-5D are a series of photographs illustrating toxicity of different concentrations of cell wall degrading enzyme applied via irrigation to Tiny Tim tomato plants. 18 day post seeding Tiny Tim plants were removed from the soil, roots cut and the plants exposed to 1 ml of 0.01 mg/ml to 1.0 mg/ml concentration of CWDE solution for 24 hours, and then replanted. Note the clear growth retardation above 0.01 mg/ml;

FIGS. 6A-6G are a series of photographs illustrating the absence of severe toxicity of different concentrations of cell wall degrading enzyme formulated in sodium phosphate and topically applied to carborundum-sprayed Tiny Tim tomato plants. Selected leaves of 18 day post seeding Tiny Tim tomato plants were sprayed with a carborundum solution, and then 100 μl of 0.1 mg/ml (FIG. 6E) to 1.0 mg/ml (FIG. 6A) CWDE in sodium phosphate topically applied. One leaf of each plant was treated (T) and one leaf untreated (C). Note lack of any significant effects on growth or vigor of the plants;

FIGS. 7A-7I are a series of photographs illustrating the effect of different concentrations of cell wall degrading enzyme (CWDE) in sodium phosphate buffer applied via irrigation to Tiny Tim tomato plants. 18 day post seeding Tiny Tim plants were removed from the soil, roots cut, dried and the plants exposed to 1 ml of 0.001 mg/ml to 1.0 mg/ml concentration of CWDE solution for 24 hours, and then replanted. Note the lack of significant growth retardation below 0.75 mg/ml;

FIGS. 8A-8D illustrate the enhanced stability of cell penetrating peptides-dsRNA complexes in the presence of the CWDE in phosphate buffered saline (PBS). KH9-BP100 peptide (SEQ ID NO: 21) and dsRNA complexes (200 molar ratio) were formed in either ddH2O (lanes 2-5) or PBS (lanes 6-9) and sampled at different time points after the addition of CWDE in different concentrations (0.1 mg/ml—lanes 2 and 6; 0.05 mg/ml—lanes 3 and 7; 0 mg/ml—lanes 4 and 8). FIG. 8A=time 0, immediately after the addition of CWDE; FIG. 8B=1 hr; FIG. 8C=2 hr; FIG. 8D=4 hr after the addition of CWDE. Lanes 5 and 9=500 ng untreated dsRNA. Note the immediate degradation of high molecular weight complexes prepared in ddH2O (lanes 2-4, FIG. 8A), and the persistence of the high molecular weight complexes prepared in PBS (lanes 6-8), up to 2 hours (FIG. 8C) after mixing with the CWDE;

FIGS. 9A-9D illustrate the enhanced stability of sodium phosphate buffer-prepared cell penetrating peptides-dsRNA complexes in the presence of the CWDE. 0.05 and 0.1 KH9-BP100 (SEQ ID NO: 21) or IR9 (SEQ ID NO: 22) peptides and dsRNA complexes (200 molar ratio) were prepared in either ddH2O or sodium phosphate buffer and sampled at different time points (FIG. 9A-time 0, FIG. 9B—1 hr, FIG. 9C—2 hr and FIG. 9D—24 hr) after the addition CWDE in different concentrations. Each time point also shows 500 ng uncomplexed dsRNA with and without treatment. Note the degradation of high molecular weight complexes prepared in ddH2O evident at 1 hour after mixing with the CWDE (FIG. 9B, see box), and the persistence of high molecular weight complexes prepared in sodium phosphate buffer at 2 hours after mixing with CWDE (FIG. 9C, boxes);

FIGS. 10A and 10B illustrate the toxicity of PBS to young plants whether applied topically to the leaves after spraying with carborundum solution or by irrigation. PBS was applied to 18 d post seeding Tiny Tim plants either topically after carborundum spray (FIG. 10A) or by irrigation (as in FIGS. 7A-7I) (FIG. 10B). Note the evidence of toxicity of PBS to the plants when applied in either manner;

FIG. 11 illustrates the absence of toxicity of sodium phosphate to young plants whether applied topically to the leaves after spraying with carborundum solution or by irrigation (as in FIGS. 10A-10B);

FIG. 12 illustrates retention of enzymatic activity of the CWDE in the presence of sodium phosphate. Tomato leaves were cut and placed overnight in 1 ml CWDE solution with 0.625M sucrose in sodium phosphate buffer with gentle agitation. CWDE activity was assessed by detection of protoplasts under low magnification. Red arrow indicates formation of protoplast, seen as green coloration of the media, in 1 mg/ml sodium phosphate;

FIG. 13 summarizes the results of irrigation of 18 day post seeding Tiny Tim tomato plants with KH9-BP100 (SEQ ID NO: 21) or IR9 (SEQ ID NO: 22) peptides and dsRNA complexes (180 or 1800 molar ratio) prepared in sodium phosphate buffer. Peptides/dsRNA complexes were administered to the Tiny Tim tomato plants with irrigation as above (See, for example, FIGS. 7A-7I) using 1 ml of complex solution with or without CWDE. 24 hr after treatment, plants were transplanted. Note the moderate toxicity evident with the KH9-BP100 peptide but not the IR9 peptide (see “Results”);

FIGS. 14A and 14B are agarose gels illustrating the stability of cell penetrating peptide-dsRNA complexes in the presence of the SK EnSpray 99 (EOS) oil. dsRNA/KH9-BP100 peptide (SEQ ID NO: 21) complexes [dsRNA/peptide molar ratios of 200 (lanes 3-6, 10 mM sodium phosphate buffer) and 2000 (lanes 7-10, 3 mM sodium phosphate buffer)] were exposed to 1% EOS oil (lanes 5, 6 and 9, 10), with (lanes 4 and 6, 8 and 10) CWDE or without the enzymes (lanes 3 and 5, 7 and 9), at two different time points: either as soon as the CWDEs were added (FIG. 14A) or 1 hr later (FIG. 14B). Lane 2 is 500 ng untreated dsRNA. Note the persistence, after 1 hour, of high molecular weight complexes in the presence of EOS mineral oil, with or without the CWDE (FIG. 14B, lanes 9 and 10);

FIGS. 15A and 15B are graphic representations of effective PDS gene silencing in tomato plants with carborundum spray and topical application of dsRNA. The indicated formulations were applied topically (100 μl/leaf) on selected leaves of 18 d post-seeding Tiny Tim tomato plants following spraying with carborundum (3 plants in each group). Treated leaves were harvested 24 hours (FIG. 14A) or 48 hours (FIG. 14B) after application and immediately frozen in liquid nitrogen for RNA extraction and qPCR analysis. PDS mRNA levels were normalized relative to actin. Note the significant reduction in PDS expression with application of the complex dsRNA+KH9 peptide (SEQ ID NO: 21)+CWDE;

FIGS. 16A and 16B are graphic representations of effective PDS and AGPase gene silencing in tomato plants with oil spray and topical application of dsRNA. The indicated formulations, comprising AGPase dsRNA (FIG. 16A) or PDS dsRNA (FIG. 16B) were applied topically (100 μl/leaf) on selected leaves of 18 d post-seeding Tiny Tim tomato plants following spraying with 1% EOS oil (3 plants in each group). Treated leaves were harvested 24 hours after application and immediately frozen in liquid nitrogen for RNA extraction and qPCR analysis. Expression levels were normalized relative to actin. “Ran” indicates dsRNA prepared against random sequences. Note the significant reduction in AGPase and PDS expression with application of the complex dsRNA+KH9 peptide (SEQ ID NO: 21)+CWDE;

FIG. 17 is a graphic representation of effective GPT silencing in citrus plants by injection of GPT dsRNA. 125 μg GPT dsRNA (or random sequence dsRNA) formulated with KHP-BP100 (SEQ ID NO: 21) (KHP-BP100:dsRNA molar ratio=2000) and 0.1 mg/ml CWDE6 per tree was injected into 6 HLB (Citrus greening) infected (experimentally infected) trees. Leaves were sampled before treatment and 7 days after treatment, frozen in liquid nitrogen for RNA extraction and qPCR analysis. GPT mRNA levels were normalized relative to elongation factor (EF-1). Note the relatively stable GPT expression in the random dsRNA recipients and nearly complete silencing (50 fold) with GPT dsRNA, delivered as described;

FIG. 18 is a graphic representation of expression of GPT in citrus in response to naked dsRNA injection. 6-10 HLB (Citrus greening) infected (experimentally infected) trees were injected with 25 mg/plant of naked (unformulated) GPT (solid squares) or naked random (solid circles) dsRNA. Leaves were sampled 15 days after treatment and frozen in liquid nitrogen for RNA extraction and qPCR analysis. GPT mRNA levels were normalized relative to elongation factor (EF-1). Note the twofold reduction in GPT expression with injection of the dsRNA.

FIG. 19 is a graphic representation of CalS expression in response to LSO infection in tomatoes. Leaves from 3-4 LSO infected (experimentally infected) plant (orange bars) and leaves from 3-4 LSO non-infected (healthy) plant (blue bars) were sampled 2, 4, 6, 8 days post infection and frozen in liquid nitrogen for RNA extraction and qPCR analysis. Cals mRNA levels were normalized relative to actin. Note the 3-4 fold upregulation in Cals levels 4-6 days post infection.

FIG. 20 is a graphic representation of disease severity index (DSI) levels in different treatment groups. Tomato plants were treated topically (100 μl/leaf, final dsRNA concentration is 100 ng/μl, molar ratio is 8400) with formulations of KH9-BP100 peptide-dsRNA complexes either with or without CWDE on selected leaves of 18 d post-seeding Tiny Tim tomato plants following spraying with 1% EOS oil (26-28 plants in each group). Then, the effect was evaluated using the DSI scoring compared to non-treated plants or plants treated with irrelevant dsRNA sequence (B2) over a period of 42 days. Note the lowest DSI levels over the period of 42 days in the yellow bar, which represents the group treated with formulation of peptide—dsCals and CWDE, compared to control groups and the group treated with formulation of peptide—dsCals, but no CWDE (dark blue bar)

FIG. 21 is a picture of representative plants from each experimental group in FIG. 20, 42 days post LSO infection. On the left, note that the plant in group C (formulation of peptide—dsCals and CWDE) which had the lowest DSI levels the disease symptoms are stunt compared to other groups and especially to the group treated with formulation of peptide—dsCals, but no CWDE (right, C compared to D)

### DESCRIPTION OF SPECIFIC EMBODIMENTS OF THE INVENTION

The present invention, in some embodiments thereof, relates to gene silencing in plant cells and plants, and, more particularly, but not exclusively, to compositions and methods for efficient delivery of nucleic acids active in RNA pathways to plant cells and plants.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

RNA interference (RNAi) pathways for gene silencing have been demonstrated in plants, providing opportunities for influencing expression of endogenous plant genes, as well as the expression of a myriad of other, both beneficial and pathogenic plant associated organisms. While transfer of dsRNA into plant cells (mainly protoplasts) has been successful in the laboratory setting, with ensuing gene silencing in many cases, widespread implementation of RNAi technology in crop plants currently awaits development of compositions and methods suitable for overcoming the formidable physical barriers unique to plants (including, but not exclusively the waxy cuticle, hardened cortex or bark, and the rigid plant cell wall).

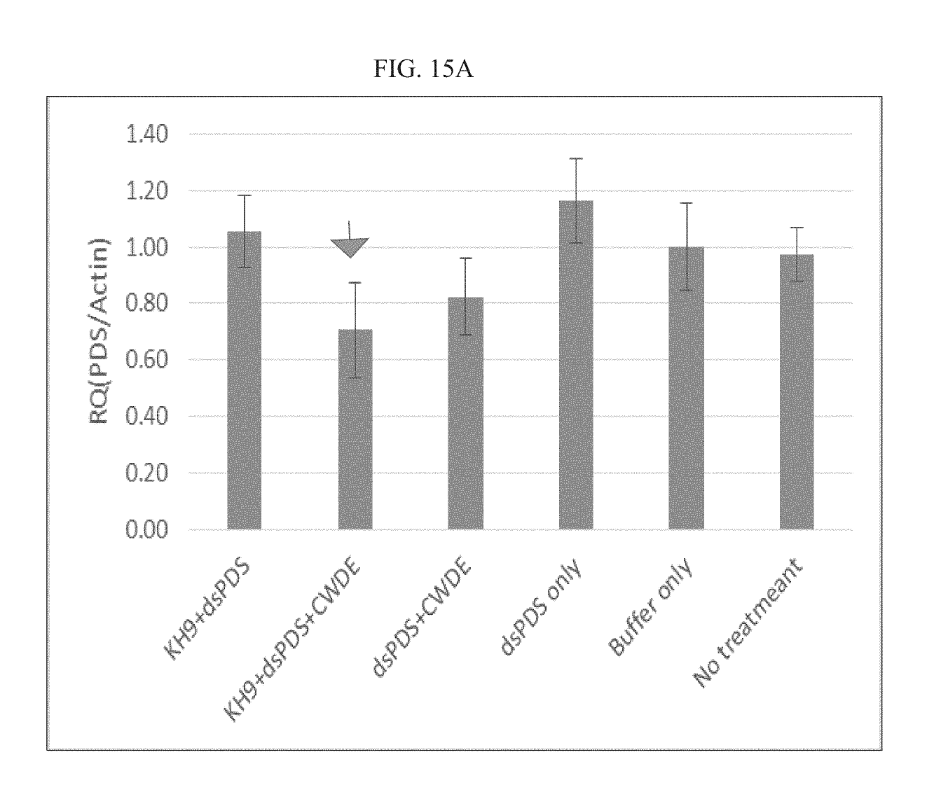

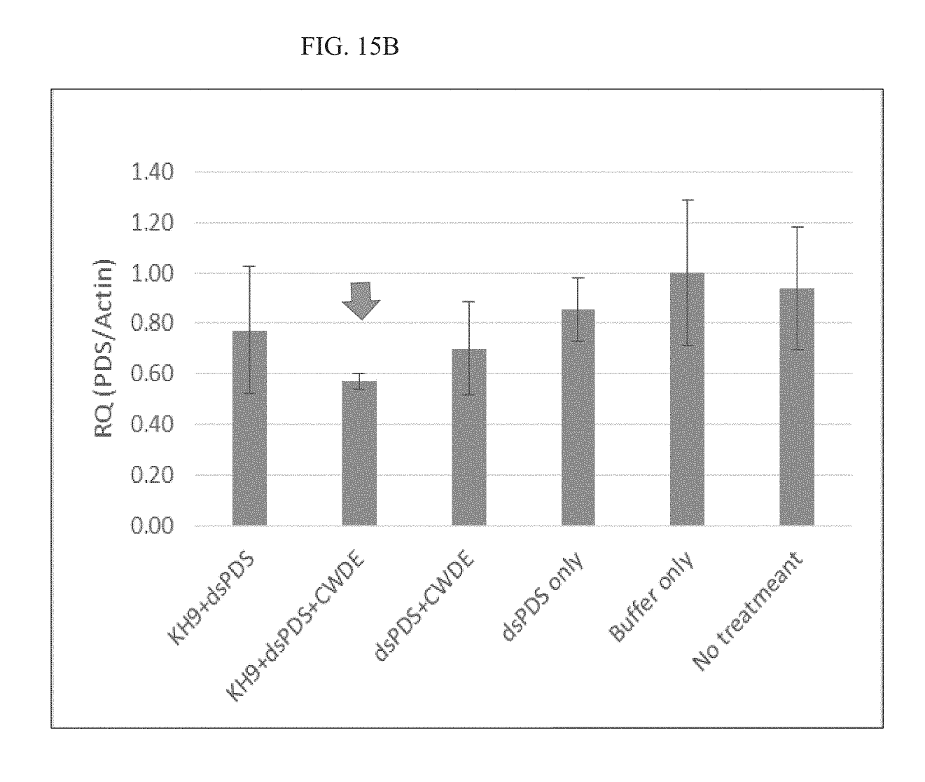

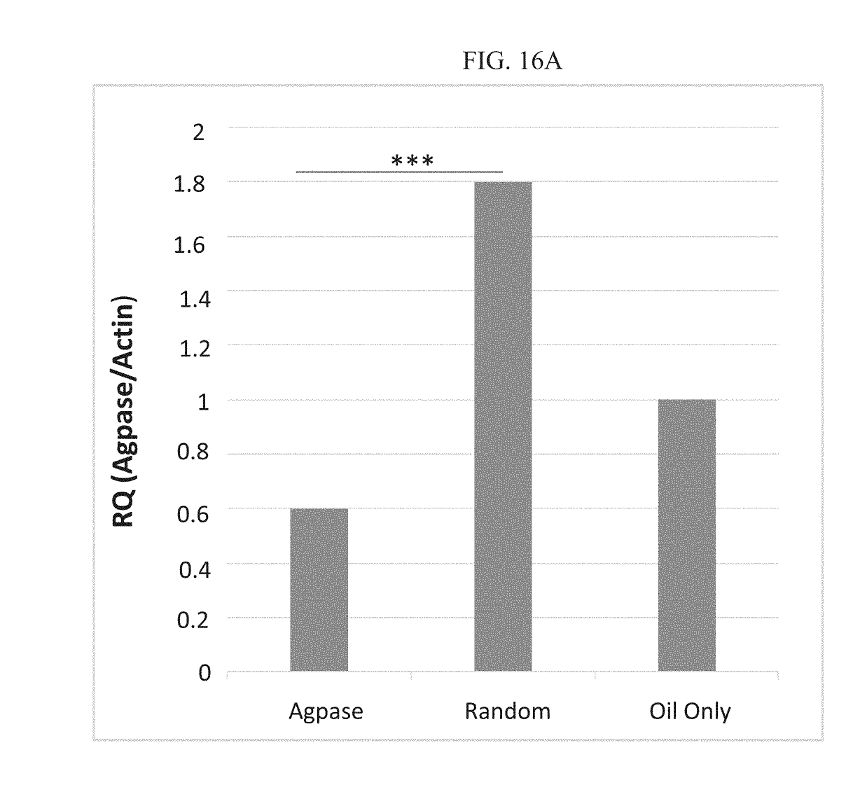

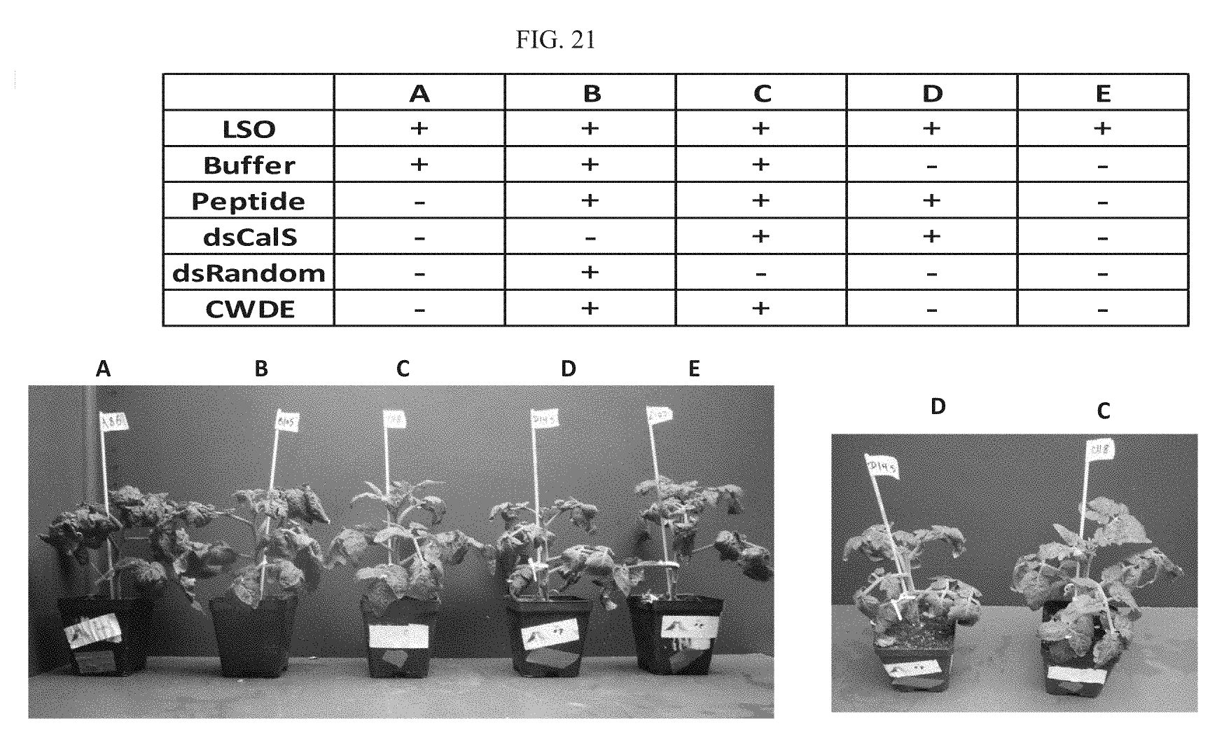

The present inventors have shown that complexing polynucleotides (e.g. dsRNA) with agents effective in facilitating transfer of polynucleotides across cell membranes, with the addition of cell wall degrading enzymes, results in a composition which can deliver dsRNA to plant cells and achieve specific and efficient gene silencing, using different methods of application, and in highly dissimilar plants (e.g. tomato as well as citrus) (see Example V and FIGS. 15-21 of the Examples section).

Thus, according to some embodiments of aspects of the invention there is provided a method of delivering a polynucleotide to a plant cell comprising contacting the plant cell with said polynucleotide and at least one cell wall degrading enzyme, and at least one of a nucleic acid condensing agent, a transfection reagent, a surfactant, and a cuticle penetrating agent. In a specific embodiment, the plant cell is a plant cell with a cell wall.

According to some embodiments of the invention, there is provided a composition of matter comprising a polynucleotide, a cell wall degrading enzyme and at least one of a nucleic acid condensing agent, a transfection reagent, a surfactant and a cuticle penetrating agent. The method of the invention can be effected using such a composition.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art. Where a term is provided in the singular, the inventors also contemplate aspects of the invention described by the plural of that term.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

For example, SEQ ID NO: 10 is expressed in a DNA sequence format (e.g., reciting T for thymine), but it can refer to either a DNA sequence that corresponds to an alpha-amylase nucleic acid sequence, or the RNA sequence of an RNA molecule (e.g., reciting U for uridine) that corresponds to the RNA sequence shown. In any event, both DNA and RNA molecules having the sequences disclosed with any substitutes are envisioned.

According to a specific embodiment, the compositions described herein are cell-free.

Polynucleotides

As used herein, “polynucleotide” refers to a nucleic acid molecule containing multiple nucleotides and generally refers both to “oligonucleotides” (a polynucleotide molecule of 18-25 nucleotides in length) and polynucleotides of 26 or more nucleotides. Embodiments of this invention include compositions including oligonucleotides having a length of 18-25 nucleotides (e.g., 18-mers, 19-mers, 20-mers, 21-mers, 22-mers, 23-mers, 24-mers, or 25-mers), or medium-length polynucleotides having a length of no fewer than 25 nucleotides and having 26 or more nucleotides (e.g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides and no greater than 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, no more than 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

Polynucleotide compositions used in the various embodiments of this invention include compositions including oligonucleotides or polynucleotides or a mixture of both, including RNA or DNA or RNA/DNA hybrids or chemically modified oligonucleotides or polynucleotides or a mixture thereof. In some embodiments, the polynucleotide may be a combination of ribonucleotides and deoxyribonucleotides, e.g., synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In some embodiments, the polynucleotide includes non-canonical nucleotides such as inosine, thiouridine, or pseudouridine. In some embodiments, the polynucleotide includes chemically modified nucleotides. Examples of chemically modified oligonucleotides or polynucleotides are well known in the art; see, e.g., Verma and Eckstein (1998) Annu. Rev. Biochem., 67:99-134. For example, the naturally occurring phosphodiester backbone of an oligonucleotide or polynucleotide can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (e.g., fluorescein or rhodamine) or other label (e.g., biotin).

The polynucleotides can be single- or double-stranded RNA (dsRNA) or single- or double-stranded DNA or double-stranded DNA/RNA hybrids or modified analogues thereof, and can be of oligonucleotide lengths or longer.

In some embodiments, the polynucleotides are dsRNA. In specific embodiments, the polynucleotide, or the dsRNA can be effective in RNA silencing (gene silencing, post-transcriptional gene silencing, “PTGS”), e.g., the dsRNA can be an RNA silencing polynucleotide.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” or “dsRNA silencing agent” refers to an RNA which is capable of specifically inhibiting or “silencing” the expression of a target gene. In certain embodiments, the dsRNA is capable of preventing complete processing (e.g, the full translation and/or expression) of an mRNA molecule through a post-transcriptional silencing mechanism. dsRNA of the invention include noncoding RNA molecules, for example RNA duplexes comprising paired strands, as well as precursor RNAs from which such small non-coding RNAs can be generated. Exemplary dsRNA include dsRNAs such as siRNAs, miRNAs and shRNAs. In one embodiment, the dsRNA is capable of inducing RNA interference. In another embodiment, the dsRNA is capable of mediating translational repression.

According to an embodiment of the invention, the dsRNA is specific to the target RNA (e.g., PDS, AGPase, etc) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or dsRNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs (siRNA) derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment, the dsRNA is greater than 30 bp. The use of long dsRNAs (i.e. dsRNA greater than 30 bp) has been very limited. However, the use of long dsRNAs can provide numerous advantages in that the cell can select the optimal silencing sequence alleviating the need to test numerous siRNAs; long dsRNAs can allow for silencing libraries to have less complexity than would be necessary for siRNAs; and, perhaps most importantly, long dsRNA could prevent viral escape mutations.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned the dsRNA of some embodiments of the invention may also be a hairpin or short hairpin RNA (shRNA).

The term “shRNA”, as used herein, refers to a dsRNA having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop abound (see, for example, Brummelkamp, T. R. et al. (2002) Science 296: 550 and Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

Synthesis of dsRNA suitable for use with some embodiments of the invention can be affected as follows. First, the target RNA sequence (e.g. mRNA sequence) is scanned downstream of the AUG start codon for AA dinucleotide sequences. Occurrence of each AA and the 3′ adjacent 19 nucleotides are recorded as potential siRNA target sites. siRNA target sites may be selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., plant, plant pathogen, etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a random nucleotide sequence or a scrambled nucleotide sequence of the siRNA is preferably used, provided it does not display any significant homology to any other gene.

It will be appreciated that the dsRNA of some embodiments of the invention need not be limited to those molecules containing only RNA, but further encompasses chemically-modified nucleotides and non-nucleotides.

In some embodiments, the dsRNA is designed to silence a gene of interest in the plant. For gene silencing by RNA interference, the dsRNA of the invention must comprise a nucleotide sequence complementary to a nucleotide sequence of the target RNA, thereby capable of hybridizing to the nucleotide sequence of the target.

The dsRNA molecule can be designed for specifically targeting a target gene of interest. It will be appreciated that the dsRNA can be used to down-regulate one or more target genes. If a number of target genes are targeted, a heterogenic composition which comprises a plurality of dsRNA molecules for targeting a number of target genes is used. Alternatively said plurality of dsRNA molecules are separately applied to the seeds (but not as a single composition). According to a specific embodiment, a number of distinct dsRNA molecules for a single target are used, which may be separately or simultaneously (i.e., co-formulation) applied.

According to an embodiment of the invention, the target gene is endogenous to the plant. Downregulating such a gene is typically important for conferring the plant with an improved, agricultural, horticultural, nutritional trait (“improvement” or an “increase” is further defined herein).

As used herein “endogenous” refers to a gene which expression (mRNA or protein) takes place in the plant. Typically, the endogenous gene is naturally expressed in the plant or originates from the plant. Thus, the plant may be a wild-type plant. However, the plant may also be a genetically modified plant (transgenic).

Downregulation of the target gene may be important for conferring improved one of, or at least one of (e.g., two of- or more), biomass, vigor, yield, fruit quality, abiotic and/or biotic stress tolerance or improved nitrogen use efficiency.

Exemplary target genes include, but are not limited to, genes which expression can be silenced to improve the yield, growth rate, vigor, biomass, fruit quality or stress tolerance of a plant. Other examples of target genes which may be subject to modulation according to the present teachings are described herein. In some embodiments, the dsRNA comprises a nucleotide sequence complementary to a sequence of Citrus sinensis magnesium-chelatase subunit ChlI, chloroplastic mRNA (SEQ ID NO: 9) Tomato GPT (tomato Glucose phosphate transporter mRNA (SEQ ID NO: 8), Citrus AGPase (citrus glucose-1-phosphate adenylyltransferase large subunit) mRNA (SEQ ID NO: 7) and Citrus CalS Solanum lycopersicum callose synthase mRNA (SEQ ID NO: 6). In another embodiment, the dsRNA is targeted to RNA sequences associated with susceptibility genes, carotenoid biosynthesis, ethylene biosynthesis, auxin biosynthesis, gibberellin biosynthesis, cytokinin biosynthesis and salicylic acid biosynthesis.

According to another embodiment, the polynucleotide may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses to humans) and have been shown to play a role in development, homeostasis, and disease etiology.

Below is a brief description of the mechanism of miRNA activity.

Genes coding for miRNAs are transcribed leading to production of a miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GPT and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Cell Wall Degrading Enzymes (CWDE)

According to some embodiments of the present invention, the plant cell having a cell wall is contacted with at least one cell wall degrading enzyme. Cell wall degrading enzymes are useful in order to facilitate contact of the polynucleotide with the plant cell membrane.

Three more or less interacting polysaccharide structures can be distinguished in the plant cell wall:

1. The middle lamella forms the exterior cell wall. It also serves as the point of attachment for the individual cells to one another within the plant tissue matrix. The middle lamella consists primarily of calcium salts of highly esterified pectins;

2. The primary wall is situated just inside the middle lamella. It is a well-organized structure of cellulose microfibrils embedded in an amorphous matrix of pectin, hemicellulose, phenolic esters and proteins;

3. The secondary wall is formed as the plant matures.

During the plant's growth and ageing phase, cellulose microfibrils, hemicellulose and lignin are deposited.

There is a high degree of interaction between cellulose, hemicellulose and pectin in the cell wall. The enzymatic degradation of these rather intensively cross-linked polysaccharide structures is not a simple process. A large number of enzymes are known to be involved in the degradation of plant cell walls. They can broadly be subdivided in cellulases, hemicellulases and pectinases. Cellulose is the major polysaccharide component of plant cell walls. It consists of beta 1,4 linked glucose polymers.

“Cellulose degrading enzymes”-Cellulose degrading enzymes include strictly processive exocellulases (cellobiohydrolases found in glycoside hydrolase) and endocellulases (properly called endo-β-1,4-glucanases), which are distributed throughout a larger number of glycoside hydrolase families, and β-Glucosidases. A feature typical for most, but not all, cellulases, and also found in some other CWDEs, is the presence a polysaccharide-binding domain connected by a loop hinge region, which aids in the binding of cellulases to their insoluble substrate.

“Hemicellulose degrading enzymes” “Hemicellulose” is a term used to describe the noncellulosic polysaccharides of the plant cell wall that comprise xyloglucans, xylans, and galactomannans. Although the linkage and sugars in the core chains are different between these major polysaccharides, the side-chain substituents often comprise the same sugar and the same linkage, and therefore the same enzymes are involved in their cleavage.

“Pectin degrading enzymes” are polygalacturonidases comprising endo- and exo-acting enzymes. Pectins are major constituents of the cell walls of edible parts of fruits and vegetables. The middle lamella which is situated between the cell walls are mainly built up from protopectin which is the insoluble form of pectin. Pectins are considered as intracellular adhesives and due to their colloidal nature they also have an important function in the water regulation system of plants. A large number of enzymes are known to degrade pectins. Examples of such enzymes are pectin esterase, pectin lyase (also called pectin transeliminase), pectate lyase, and endo- or exo-polygalacturonase (Pilnik and Voragen (1990). Food Biotech 4, 319-328). Apart from enzymes degrading smooth regions, enzymes degrading hairy regions such as rhamnogalacturonase and accessory enzymes have also been found (Schols et al. (1990), Carbohydrate Res. 206, 105-115; Searle Van Leeuwen et al. (1992). Appl. Microbiol. Biotechn. 38, 347-349). Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by the Enzyme Nomenclature (1992) such as pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82). Pectate lyases degrade un-methylated (polygalacturonate) or low-methylated pectin by beta-elimination of the alpha-1,4-glycosidic bond. The enzymes are generally characterized by an alkaline pH optimum, an absolute requirement for Ca²⁺ (though its role in binding and catalysis is unknown) and good temperature stability.

“Side-Chain Cleaving and Other Accessory Enzymes” In addition to the enzymes described above, additional enzymes are required to cleave the linkage to side chains, to remove modifications (such as methylesters and acetylation).

A non-limiting list of cell wall degrading enzymes suitable for use with the instant invention, their pH optima, and known substrates is provided in Table 1:

TABLE 1
(from Bauer, PNAS 2006, 103: 11417)

| | | | | | |
|---|---|---|---|---|---|
| | Accession | | Tₒₚₜ, | | |
| Enzyme | no. | pHₒₚₜ | ° C. | Activity* | Known substrates |

Active on glucans

| | | | | | |
|---|---|---|---|---|---|
| Endo-β(1,4)-glucanase | AN1602.2 | <6 | ND | 1.12 | Soluble CMC † |
| Endo-β(1,4)-glucanase | AN5214.2 | 4.0 | 52 | 2.85 | Soluble CMC, † cellooligos Glc₄ |
| | | | | | (slow)/Glc₅/Glc₆ |
| Endo-β(1,4)-glucanase | AN1285.2 | 4.0 | 57 | 5.30 | Soluble CMC, † cellooligos |
| | | | | | Glc₄/Glc₅/Glc₆, barley β-glucan and |
| | | | | | lichenan |
| Endo-β(1,4)-glucanase | AN3418.2 | 5.5 | 42 | 1.85 | CMC, † cellooligos Glc₄/Glc₅/Glc₆, |
| | | | | | barley β-glucan and lichenan and |
| | | | | | tamarind XG |
| Cellobiohydrolase | AN0494.2 | ND | ND | 1.82 | Soluble CMC, † cellooligos Glc₃ |
| | | | | | (slow)/Glc₄/Glc₅/Glc₆ |
| Cellobiohydrolase | AN5282.2 | 5.5 | 57 | 2.40 | Soluble CMC, † cellooligos |
| | | | | | Glc₃/Glc₄/Glc₅/Glc₆, barley β- |
| | | | | | glucan and lichenan, avicel |
| Cellobiohydrolase | AN5176.2 | ND | ND | ND | ND |
| β-Glucosidase | AN2227.2 | ND | ND | ND | ND |
| β-Glucosidase | AN2612.2 | ND | ND | ND | ND |
| β-Glucosidase | AN0712.2 | 5.5 | 52 | A | PNP-β-glucoside † |
| β-Glucosidase | AN1551.2 | ND | ND | ND | Not active on PNP-β-glucoside |
| β-Glucosidase | AN1804.2 | 6.0 | 52 | 87.84 | PNP-β-glucoside † |
| α-/β-Glucosidase | AN7345.2 | ND | ND | 1.66 | PNP-β-glucoside † and PNP-α- |
| | | | | | glucoside |
| Mixed-linked glucanase | AN2385.2 | ND | ND | 2.33 | Laminarin, † lichenan, soluble CMC, |
| | | | | | not on pustulan |
| Endo-β(1,3)-glucanase | AN4700.2 | ND | ND | 1.35 | Laminarin † lichenan |
| Exo-β(1,3)-glucanase | AN7533.2 | ND | ND | 0.58 | Lichenan † |
| Endo-β(1,3)-glucanase | AN7950.2 | ND | ND | ND | ND |
| β-(1,6)-Glucanase | AN3777.2 | ND | ND | 0.15 | Pustulan (β-1,6-glucan) † |

Active on xyloglucans

| | | | | | |
|---|---|---|---|---|---|
| XG-specific | AN0452.2 | 6.5 | 47 | 1.97 | Tamarind XG † |
| endoglucanase | | | | | |
| Oligoxyloglucan | AN1542.2 | 3.0 | 42 | 0.08 | Tamarind XG, † tamarind XG |
| reducing | | | | | oligomers |
| End-specific | | | | | |
| xyloglucanase | | | | | |
| α-Fucosidase | AN8149.2 | ND | ND | A | Cotton XG oligomers, not active on |
| | | | | | PNP-fucoside |
| α-Xylosidase | AN7505.2 | ND | ND | 0.21 | PNP-α-xyloside † |

Active on xylans

| | | | | | |
|---|---|---|---|---|---|
| Endo-β(1,4)-xylanase | AN1818.2 | 4.9 | 52 | 51.16 | LWX †, BWX, RAX, OSX, Xyl₄ and |
| | | | | | Xyl₆ |
| Endo-β(1,4)-xylanase | AN3613.2 | 5.4 | 52 | 18.83 | LWX †, BWX, RAX, OSX, Xyl₄ and |
| | | | | | Xyl₆ |
| β-Xylosidase | AN2359.2 | 5.1 | 52 | 29.00 | PNP-β-xyloside, † Xyl₆ |
| β-Xylosidase/α- | AN8401.2 | 4.4 | 48 | 0.28 | RAX † and Xyl₆, not on PNP-β- |
| arabinosidase | | | | | xyloside |
| Acetylxylan esterase | AN3294.2 | ND | ND | 2.33 | Naphthyl acetate, PNP-acetate † |
| Acetylxylan esterase | AN6093.2 | 7.5 | 49 | 33.33 | Naphthyl acetate, PNP-acetate † |
| Ferulic acid esterase | AN5267.2 | 6.1 | 37 | 0.03 | PNP-acetate, methyl ferulate, † |
| | | | | | wheat arabinoxylan |
| α-Glucuronidase | AN9286.2 | 4.0 | 30 | 0.02 | LWX, † APTS labeled 4-O-methyl |
| | | | | | glucuronosyl Xyl₃ |

Active on mannans

| | | | | | |
|---|---|---|---|---|---|
| Endo-β(1,4)-mannanase | AN3297.2 | ND | ND | 0.67 | LBG, † GG |
| Endo-β(1,4)-mannanase | AN3358.2 | 5.5 | 52 | 2.13 | LBG, † GG |
| Endo-β(1,4)-mannanase | AN6427.2 | ND | ND | 2.05 | LBG, † GG |
| Endo-β(1,4)-mannanase | AN7413.2 | ND | ND | ND | ND |
| β-Mannosidase | AN3368.2 | ND | ND | 3.22 | PNP-β-mannoside † |
| α-Galactosidase | AN7152.2 | 5.0 | 52 | 1.50 | PNP-α-galactoside †; not on |
| | | | | | raffinose, LBG, or GG |
| α-Galactosidase | AN7624.2 | ND | ND | ND | Not active on PNP-α-galactoside, |
| | | | | | raffinose |
| α-Galactosidase | AN8138.2 | 3.5 | 52 | 42.17 | PNP-α-galactoside, † raffinose, |
| | | | | | LBG, GG |
| α-Galactosidase | AN9035.2 | ND | ND | ND | Not active on PNP-α-galactoside, |
| | | | | | raffinose |

Active on pectin

| | | | | | |
|---|---|---|---|---|---|
| Pectin lyase | AN2331.2 | 7.0 | ND | 3.50 | Citrus pectin † |
| Pectin lyase | AN2569.2 | ND | ND | 5.00 | Citrus pectin † |
| Pectate lyase | AN0741.2 | 8.5 | 37 | 0.67 | Pectic acid † |
| Pectate lyase | AN3337.2 | 9.2 | 37 | 11.33 | Pectic acid † |
| Pectate lyase | AN7646.2 | 8.5 | 22 | 1.17 | Pectic acid † |
| Pectate lyase | AN8453.2 | 7.8 | 22 | 1.83 | Pectic acid † |
| Endo-polygalacturonase | AN4372.2 | 5.1 | 38 | 21.42 | Pectic acid, † less active on citrus |
| | | | | | pectin |
| Endo-polygalacturonase | AN8327.2 | 4.8 | 48 | 18.58 | Pectic acid, † less active on citrus |
| | | | | | pectin |
| Exo-polygalacturonase | AN8761.2 | 4.4 | 48 | 25.00 | Pectic acid, † less active on citrus |
| | | | | | pectin |
| Exo-polygalacturonase | AN9045.2 | ND | ND | A | GalA oligomers |
| Pectin methyl esterase | AN3390.2 | 8.0 | 30 | 10.00 | Citrus pectin † |
| Rhamnogalacturonase | AN9134.2 | ND | ND | 0.58 | Linseed RG † |
| Rhamnogalacturonan | AN6395.2 | ND | ND | 1.15 | Linseed RG † |
| lyase | | | | | |
| Rhamnogalacturonan | AN7135.2 | ND | ND | 0.55 | Linseed RG † |
| lyase | | | | | |
| Rhamnogalacturonan | AN2528.2 | ND | ND | 11.67 | PNP-acetate, † naphthyl acetate |
| Acetylesterase α-L- | AN10277.3 | 5.0 | ND | A | RG oligomers, not on PNP-α-L- |
| rhamnosidase | | | | | rhamnoside, not on naringin or |
| | | | | | hesperidin |
| Endo-α(1,5)- | AN6352.2 | ND | ND | 0.17 | Debranched arabinan † |
| arabinosidase | | | | | |
| Endo-α(1,5)- | AN8007.2 | ND | ND | 0.02 | Debranched arabinan † |
| arabinosidase | | | | | |
| Endo-α(1,5)- | AN3044.2 | ND | ND | ND | ND |
| arabinosidase | | | | | |
| α-L- | AN1571.2 | 4.8 | 65 | 23.50 | PNP-α-arabinofuranoside, † sugar |
| arabinofuranosidase | | | | | beet arabinan, Ara₇ |
| α-L- | AN7908.2 | 5.4 | 47 | 7.83 | PNP-α-arabinofuranoside, † sugar |
| arabinofuranosidase | | | | | beet arabinan, RAX |
| α-L- | AN1277.2 | ND | ND | A | Ara₇ |
| arabinofuranosidase | | | | | |
| Endo-β(1,4)-galactanase | AN5727.2 | 5.0 | ND | 66.58 | Potato pectic galactan † |
| Xylogalacturonase | Afu8g06890 | ND | ND | A | Water melon xylogalacturonan |
| β-Galactosidase | AN3201.2 | ND | ND | ND | ND |
| Miscellaneous | | | | | |
| β-Glucuronidase | AN5361.2 | ND | ND | ND | ND |
| Cutinase | AN7541.2 | ND | ND | 304.17 | PNP-butyrate † |
| Cutinase | AN7180.2 | ND | ND | 1.33 | PNP-butyrate † |
| α-Glucosidase | AN0941.2 | 5.5 | 52 | 0.22 | PNP-α-glucoside † |
| α-Glucosidase | AN4843.2 | ND | ND | ND | Not active on PNP-α-glucoside |
| Endo-β-(1,6)- | NCU09702.1 | ND | ND | ND | ND |
| galactanase | | | | | |
| α-1,2-Mannosidase | AN0787.2 | ND | ND | ND | Not active on PNP-α-mannoside |
| α-1,2-Mannosidase | AN3566.2 | ND | ND | ND | Not active on PNP-α-mannoside |
| α-1,3-Glucanase | AN7349.2 | ND | ND | 0.40 | α-1,3-Glucan (mutan) † from A. nidulans |
| (mutanase) | | | | | |
| N,O- | AN6470.2 | 4.0 | 24 | 4510.0 ‡ | Dried micrococcus cells (bacterial |
| diacetylmuramidase | | | | | cells) † |

It will be appreciated that the compositions and methods of the present invention are not limited to the cell wall degrading enzymes of Table 1. In some embodiments, the cell wall degrading enzymes are selected from the group consisting of cellulases, hemicellulases, lignin-modifying enzymes, cinnamoyl ester hydrolases and pectin-degrading enzymes. Considering the complexity of cell wall structure, as detailed above, it is possible that efficient cell wall penetration can require more than one cell wall degrading enzyme. Thus, in some embodiments, the at least one cell wall degrading enzyme comprises a combination of cell wall degrading enzymes with distinct substrate specificities, for example, a combination of cellulases, pectinases and hemicellulases, or any other of the enzymes in Table 1. In a particular embodiment, the at least one cell wall degrading enzyme comprises a combination of cellulases, xylases and laminarinases such as, for example, Drisilase™ (Sigma Cat No. D9519, Sigma Chemicals, St Louis, Mo.).

Cell wall degrading enzymes can be detrimental to plants, indeed, are most typically used in the paper and tree-product industry in decomposition of woody materials, and they should be tested for toxicity when prepared for the compositions and methods of the present invention. Toxicity can be evaluated by contacting plants with increasing concentrations of the CWDE and determining vigor and growth (coloration, turgor, etc) of the plant. It will be appreciated that CWDE concentrations suitable for use with the invention will typically be below those concentrations familiar from other industrial use of CWDE. In some embodiments, the CWDE (e.g. Drisilase, Sigma Chemical, St. Louis Mo.) is provided in a sodium phosphate buffer (pH 6.8) at a concentration range of 0.001 to 50 mg/ml, 0.005 to 20 mg/ml, 0.1 to 10 mg/ml, 0.1 to 5 mg/ml, 0.1, 0.5, 1.0 or 2.0 mg/ml. In a specific embodiment, the CWDE is provided at either 0.1 or 1 mg/ml. Further, conditions for optimum CWDE activity can be determined by assaying the release of protoplasts from plant structures (e.g. leaves) using candidate CWDE, buffers and pH ranges (see Example IV of the Examples section hereinbelow).

It will be further appreciated that the effect of CWDE on target plants can vary with mode of application. The inventors have found that, with tomato plants, no toxicity of CWDE to the plants was noted when applied topically or via irrigation, at a concentration of up to 0.75 mg/ml. Thus, in some embodiments, CWDE are provided via irrigation, at concentrations in the range of 0.1-0.75 mg/ml, 0.2-0.5 mg/ml or 0.3-0.4 mg/ml.

The inventors have found that, in some formulations, and at some concentrations, the presence of CWDE has a destabilizing effect on the dsRNA-cell penetrating peptide complex. Thus, in some embodiments, CWDE is mixed with the peptide:dsRNA complex immediately before, or a few (e.g. 5-30) minutes before application of the peptide:dsRNA complex to the plant.

In some embodiments, the action of cell wall degrading enzymes can be enhanced by incorporating additional cell-wall active agents, such as expansins (e.g. swollenin), cell wall extensibility factors capable of “relaxing” cell wall architecture (for a review see Peaucelle, Front Plant Sci 2012 3; art 121).

Further, it will be appreciated that delivering the polynucleotide to some families and species of plants, plant structures or organs (seeds, leaves, etc), or plants at specific stages of their life cycle (shoots v stems, etc), having individually characteristic cell wall composition, may require specially formulated cell wall degrading enzymes or combinations thereof, and that treatment of plants (for example, crop plants) according to the method of the invention may require use of different compositions at different stages of growth of the plant or crop.

Nucleic Acid Condensing Agents

Bioactive macromolecules, and nucleic acids and polynucleotides in particular, are typically large in size, and carry a significant charge (due mostly to the negative ribose-phosphate backbone), therefore making transport of the polynucleotides into cells, via the lipophilic and hydrophobic cell membrane a major undertaking. One approach to facilitating the transfer of polynucleotides into the cell is to condense the polynucleotide mass, using a condensing agent, or agents. Thus, in some embodiments, the compositions and methods of the present invention can comprise a nucleic acid condensing agent or agents.

As used herein, the term “nucleic acid condensing agent” refers to any agent which interacts with a nucleic acid (e.g. DNA, RNA) to reduce the volume occupied by the nucleotide in a solution. Highly effective nucleotide condensing agents can reduce the nucleic acid to a compact state in which the volume fractions of the solvent and the nucleic acid in solution are comparable. Entities capable of inducing DNA condensation are numerous, including small molecules (e.g. multivalent cations and cationic lipids), polymeric materials (e.g. linear and branched polymers and dendrimers), biomolecules (e.g., peptides and proteins), and nanomaterials (e.g. nanoparticles and carbon nanotubes).

Some exemplary nucleic acid condensing agents include, but are not limited to, cations of charge +3 or greater, such as the naturally occurring polyamines spermidine3+ and spermine4+ (Chattoraj et al., 1978; Gosule & Schellman, 1976) and the inorganic cation hexamine cobalt [Co(NH₃)₆³⁺], cationic polypeptides such as polylysine and polyarginine (Laemmli, 1975), and basic proteins such as histones H1 and H5. Under specific circumstances (water-alcohol mixture), divalent metal cations can also provoke condensation in water at room temperatures in water-alcohol mixtures. Alcohols and neutral or anionic polymers can also provoke polynucleotides condensation (high concentrations of ethanol are commonly used to precipitate DNA, but under carefully controlled conditions it can produce particles of well-defined morphology). Co(NH3)6³⁺ added to ethanol at low ionic strength, acts synergistically. Neutral polymers such as PEG, at high concentrations and in the presence of adequate concentrations of salt produce condensation of polynucleotides. Similar condensation is also produced by anionic polymers, such as polyaspartate, polyglutamate, and the anionic peptides found in the capsid of bacteriophage T4.

Thus, exemplary nucleic acid condensing agents suitable for use in the methods and compositions of the present invention include, but are not limited to protamine, spermidine3+, spermine4+, hexamine cobalt, polycationic peptides such as polylysine and polyarginine, histones H1 and H5 and polymers such as PEG, polyaspartate and polyglutamate. It will be noted that condensation conditions can vary with the size of the polynucleotide (greater condensation with polynucleotides a few hundred bases/base pairs or more), and with pH, ionic strength and other characters of the solution. Typically, for example, spermidine or spermine is added at a concentration of about 100, about 200, about 300 to about 500 μM for effective condensation.

Transfection Regents

In some embodiments, a component of the complexes used in the present invention is a transfection agent. As used herein, the term “transfection reagent” or “transfection agent” refers to an agent effective in facilitating entry of biological molecules, and specifically large, charged biomolecules such as polynucleotides into cells. Suitable transfection agents in the context of the present invention include cationic and polycationic polymers or particles (such as calcium phosphate, gold, silica, carbon nanotubes, quantum dots), and/or cationic and polycationic lipids.

Cationic and polycationic polymers suitable for use in the invention are known in the art and include, for example, linear and branched polysaccharides, dense star dendrimers, PAMAM dendrimers, NH3 core dendrimers, ethylenediamine core dendrimers, dendrimers of generation 5 or higher, dendrimers with substituted groups, dendrimers comprising one or more amino acids, grafted dendrimers and activated dendrimers, polyethyleneimine, polyethyleneimine conjugates, and polyalkylenimine.

In some embodiments, the transformation agent can be a cell penetrating peptide (CPP). CPPs are commonly able to efficiently pass through cell membranes while carrying a wide variety of cargos inside cells. CPP sequences are known to vary considerably; however, several similarities exist between the structural nature of these short peptides. Almost every CPP sequence involves positively charged amino acids: in fact, a chain of arginines forms one of the most widely used CPPs. The membranolytic properties of a given CPP can also be governed by its secondary structure, specifically, it has been shown that peptides with an R-helical region can more efficiently enter cells. Some commonly used CPPs, and modifications that can enhance their efficiency are described in detail in Copolovici et al, (ACS Nano, 2014, 8:1972).

A specific example of cell penetrating peptide modification includes CPPs combined with a polycation moiety (see, for example, Namura et al, 2014). Exemplary peptides which have been shown to be effective in facilitating transfer of dsRNA to plant cells in the methods and compositions of the invention include (KH)9-BP100 (KHKHKHKHKHKHKHKHKHKKLFKKILKYL-NH 2, SEQ ID NO: 21) and IR9 (GLFEAIEGFIENGWEGMIDGWYGRRRRRRRRR)(SEQ ID NO: 22).

Combination of the polynucleotide with cell penetrating peptide transforming agents, in order to effectively condense and aid transformation of the polynucleotide (e.g. dsRNA) will be most effective at a specific range of transforming agent (peptide):polynucleotide (dsRNA), which can be generalized for a number of transforming agent:dsRNA combinations, or may be unique for an individual or small group of combinations. The present inventors have found that, using peptides (KH)9-Bp100 and IR9, stability of the peptide:dsRNA complex was significantly improved at peptide:dsRNA molar ratios greater than 100, in the range of 200-2000. Thus, in some embodiments, effective cell penetrating peptide:dsRNA molar ratio is in the range of 10 to 10,000, 50 to 5000, 75 to 4000, 100 to 3000, 150 to 2000, 200 to 2000, 250 to 1500, about 10, about 25, about 50, about 100, about 150, about 200, about 250, about 300, about 350, about 400, about 450, about 500, about 550, about 600, about 650, about 700, about 800, about 900, about 1000, about 1100, about 1200, about 1300, about 1400, about 1500, about 1600, about 1700, about 1800, about 1900, about 2000, about 2100, about 2300, about 2500, about 2750, about 3000, about 4000, about 5000, about 6000, about 7000, about 8000, about 10,000. In a specific embodiment, the peptide:dsRNA molar ratio is 200 or 2000.

It will be noted that the present invention is not limited to use of these polycationic polymer transfection agents.

In some embodiments, the transfection agent is a lipid, for example, a cationic lipid (or a mixture of a cationic lipid and neutral lipid). The lipid can be used to form a peptide- or protein-nucleic acid-lipid aggregate which facilitates introduction of the anionic nucleic acid through cell membranes. Transfection compositions of this invention comprising peptide- or protein-nucleic acid complexes and lipid can further include other non-peptide agents that are known to further enhance transfection.

Inclusion of a peptide- or protein-nucleic acid complex or a modified peptide- or protein-nucleic acid complex in a cationic lipid transfection composition can significantly enhance transfection (often by 2-fold or more, and in some cases by over 30 fold) of the nucleic acid compared to transfection of the nucleic acid mediated by the cationic lipid alone.

Monovalent or polyvalent cationic lipids can be employed in cationic lipid transfecting compositions. Illustrative monovalent cationic lipids include DOTMA (N-[1-(2.3-dioleoyloxy)-propyl]-N,N,N-timethyl ammonium chloride), DOTAP (1,2-bis(oleoyloxy)-3-3-(trimethylammonium)propane), DMRIE (1,2-dimyristyloxypropyl-3-dimethyl-hydroxy ethyl ammonium bromide), DDAB (dimethyl dioctadecyl ammonium bromide), DC-Chol (3-(dimethylaminoethane)-carbamoyl-cholestrerol). Suitable polyvalent cationic lipids are lipospermines, specifically, DOGS (Dioloctadecylaminoglycyl spermine), DOSPA (2,3-dioleyloxy-N-[2(sperminecarboxamido)ethyl]-N,N-dimethyl-1-propanamin-ium trifluoroacetate) and DOSPER (1,3-dioleoyloxy-2-(6carboxy spermyl)-propyl-amid; N-1-dimethyl-N-1-(2,3-dialkyloxypropyl)-2-hydroxypropane-1,3-diamine including but not limited to N-1-dimethyl-N-1-(2,3-diaoleoyloxypropyl)-2-hydroxypropane-1,3-diamine, N-1-dimethyl-N-1-(2,3-diamyristyloxypropyl)-2-hydroxypropane-1,3-diamine, N-1-dimethyl-N-1-(2,3-diapalmityloxypropyl)-2-hydroxypropane-1,3-diamine; N-1-dimethyl-N-1-(2,3-dialkyloxypropyl)-2-(3-amino-2-hydroxypropyloxy)propane-1,3-diamine including but not limited to N-1-dimethyl-N-1-(2,3-diaoleoyloxypropyl)-2-(3-amino-2-hydroxypropyloxy)propane-1,3-diamine, N-1-dimethyl-N-1-(2,3-diamyristyloxypropyl)-2-(3-amino-2-hydroxypropyloxy)propane-1,3-diamine, N-1-dimethyl-N-1-(2,3-diapalmityloxypropyl)-2-(3-amino-2-hydroxypropyloxy)propane-1,3-diamine; and the di- and tetra-alkyl-tetra-methyl spermines, including but not limited to TMTPS (tetramethyltetra-palmitoyl spermine), TMTOS (tetramethyltetraoleyl sp. ermine), TMTLS (tetramethlytetralauryl spermine), TMTMS (tetramethyltetramyristyl spermine) and TMDOS (tetramethyldioleyl spermine); and 1,4,-bis[(3-amino-2-hydroxypropyl)-alkylamino]-butane-2,3-diol including but not limited to 1,4,-bis[(3-amino-2-hydroxypropyl)-oleylamino]-butane-2,3-diol, 1,4,-bis[(3-amino-2-hydroxypropyl)-palmitylamino]-butane-2,3-diol, 1,4,-bis[(3-amino-2-hydroxypropyl)-myristylamino]-butane-2,3-diol; and 1,4-bis(3-alkylaminopropyl)piperazine including but not limited to 1,4-bis[(3-oleylamino)propyl]piperazine, 1,4-bis[(3-myristylamino)propyl]piperazine, 1,4-bis[(3-palmitylamino)propyl]piperazine; and a 1,4-bis[(3-(3-aminopropyl)-alkylamino)propyl)piperazine including but not limited to 1,4-bis[(3-(3-aminopropyl)-oleylamino)propyl]piperazine, 1,4-bis[(3-(3-aminopropyl)-myristylamino)propyl]piperazine, 1,4-bis[(3-(3-aminopropyl)-palmitylamino)propyl]piperazine; and 1,4-bis[(3-(3-amino-2-hydroxypropyl)-alkylamino)propyl]piperazine including but not limited to 1,4-bis[(3-(3-amino-2-hydroxypropyl)-oleylamino)propyl]piperazine, 1,4-bis[(3-(3-amino-2-hydoxypropyl)-myristylamino)propyl]piperazine, 1,4-bis[(3-(3-amino-2-hydroxypropyl)-palmitylamino)propyl]piperazine, 1,4-bis[(3-(3-aminopropyl)-alkylamino)-2-hydroxypropyl]piperazine including but not limited to 1,4-bis[(3-(3-aminopropyl)-oleylamino)-2-hydroxy-propyl]piperazine, I,4-bis[(3-(3-aminopropyl)-myristylamino)-2-hydroxypropyl]piperazine, 1,4-bis[(3-(3-aminopropyl)-palmitylamino)-2-hydroxy-propyl]piperazine.

In certain illustrative examples the cationic lipids that may be used include the commercial agents LipofectAmine™ 2000, LipofectAmine™, Lipofectin®, DMRIE-C, CellFectin® (Invitrogen), Oligofectamine® (Invitrogen), LipofectAce® (Invitrogen), Fugene® (Roche, Basel, Switzerland), Fugene® HD (Roche), Tranffectam® (Tranfectam, Promega, Madison, Wis.), Tfx-10® (Promega), TN-20® (Promega), Tfx-50® (Promega), Transfectin™ (BioRad, Hercules, Calif.), SilentFect™ (Bio-Rad), Effectene® (Qiagen, Valencia, Calif.), DC-chol (Avanti Polar Lipids), GenePorter® (Gene Therapy Systems, San Diego, Calif.), DharmaFect I® (Dharmacon, Lafayette, Colo.), DharmaFect 2® (Dharmacon), DharmaFect 3® (Dharmacon), DharmaFect 4® (Dharmacon), Escort™ III (Sigma, St. Louis, Mo.) and Escort™ IV (Sigma).

Cationic lipids can also be combined with non-cationic lipids, particularly neutral lipids, for example lipids such as DOPE (dioleoylphosphatidylethanolamine), DPhPE (diphytanoylphosphatidylethanolamine) or cholesterol. The ratio can vary from 1:1 (molar) to 4:1 (molar) of cationic to neutral lipids.

Exemplary transfection compositions include those which induce substantial transfection of a plant cells. Inclusion of a peptide- or protein-nucleic acid or modified peptide- or protein-nucleic acid complex in a polycationic polymer transfection composition may significantly enhance transfection.

Transfection Enhancing Agents

The complexes formed between the polynucleotide, the cell wall degrading enzyme, with or without additional transfection agent may be further enhanced by inclusion of moieties such as proteins or peptides that function for nuclear or other sub-cellular localization, function for transport or trafficking, are receptor ligands, comprise cell-adhesive signals, cell-targeting signals, cell-internalization signals, endocytosis signals, or even cell penetration signals as nucleic acid sequences encoding one or more protein chains.

Surfactants

Surfactants can be employed in the methods and compositions of the present invention. Surfactants may aid in penetrating waxy cuticle or bark of some plants and plant structures, can aid in “spreading” topically applied liquids on plant surfaces and may facilitate access of the complexed polynucleotide-cell wall degrading enzyme to the cell wall of target plant cells.

As used herein, the term “surfactant” refers to any compound or composition that acts to lower the surface tension (or interfacial tension) between two liquids or between a liquid and a solid. Surfactants can be, inter alia, wetting agents, emulsifiers, foaming agents and dispersants, and are commonly divided into anionic surfactants (negative charge), cationic surfactants (positive charge) and amphoteric surfactants (both positive and negative charges).

Exemplary surfactants used in agriculture, that can be used with the methods and compositions of the present invention include, but are not limited to alkyl glucosides, amino acid based surfactants, ascorbic based surfactants, carbohydrate based surfactants, carbohydrate esters, cellulose ether surface active polymers, fatty amide surfactants, insulin based surface active polymers, lactic acid surfactants, lignosulfonates, lysine based surfactants, nitrogen based surfactants, phospholipids, polar lipid based surfactants, polyethylene glycol fatty acid esters, polyglycerol fatty acid esters, protein based surfactants, rhamnolipids, saponins, sophorlipids and sterol ethoxylates. In one specific embodiment, the surfactant is a lecithin, and more specifically, a soy lecithin.

Specific surfactants suitable for in the present invention are not particularly limited, and examples of the surfactants can be grouped into the following (A), (B), and (C). These may be used singly or in combination.

(A) Nonionic surfactants: A measurement frequently used to describe surfactants is the HLB (hydrophilic/lipophilic balance). The HLB describes the ability of the surfactant to associate with hydrophilic and lipophilic compounds. Surfactants with a high HLB balance associate better with water soluble compounds than with oil soluble compounds. Herein, the HLB value should be 12 or greater, or at least 13. As noninionic surfactants, organo-silicone surfactants such as polyalkyleneoxide-modified heptamethyltrisiloxane are suitable for the present invention. A commercial product is Silwet L77™ spray adjuvant from GE Advanced Materials.

(A-1) Polyethylene glycol type surfactants: examples of polyethylene glycol type surfactants include polyoxyethylene alkyl (C12-18) ether, ethylene oxide adduct of alkylnaphthol, polyoxyethylene (mono or di) alkyl (C8-12) phenyl ether, formaldehyde condensation product of polyoxyethylene (mono or di) alkyl (C8-12) phenyl ether, polyoxyethylene (mono, di, or tri) phenyl phenyl ether, polyoxyethylene (mono, di, or tri) benzyl phenyl ether, polyoxypropylene (mono, di, or tri) benzyl phenyl ether, polyoxyethylene (mono, di, or tri) styryl phenyl ether, polyoxypropylene (mono, di or tri) styryl phenyl ether, a polymer of polyoxyethylene (mono, di, or tri) styryl phenyl ether, a polyoxyethylene polyoxypropylene block polymer, an alkyl (C12-18) polyoxyethylene polyoxypropylene block polymer ether, an alkyl (C8-12) phenyl polyoxyethylene polyoxypropylene block polymer ether, polyoxyethylene bisphenyl ether, polyoxyethylene resin acid ester, polyoxyethylene fatty acid (C12-18) monoester, polyoxyethylene fatty acid (C12-18) diester, polyoxyethylene sorbitan fatty acid (C12-18) ester, ethylene oxide adduct of glycerol fatty acid ester, ethylene oxide adduct of castor oil, ethylene oxide adduct of hardened caster oil, ethylene oxide adduct of alkyl (C12-8) amine and ethylene oxide adduct of fatty acid (C12-18) amide;

(A-2) Polyvalent alcohol type surfactants: examples of polyvalent alcohol type surfactants include glycerol fatty acid ester, polyglycerin fatty acid ester, pentaerythritol fatty acid ester, sorbitol fatty acid (C12-18) ester, sorbitan fatty acid (C12-8) ester, sucrose fatty acid ester, polyvalent alcohol alkyl ether, and fatty acid alkanol amide;

(A-3) Acetylene-type surfactants: examples of acetylene type surfactants include acetylene glycol, acetylene alcohol, ethylene oxide adduct of acetylene glycol and ethylene oxide adduct of acetylene alcohol.

(B) Anionic Surfactants:

(B-1) Carboxylic acid type surfactants: examples of carboxylic acid type surfactants include polyacrylic acid, polymethacrylic acid, polymaleic acid, a copolymer of maleic acid and olefin (for example, isobutylene and diisobutylene), a copolymer of acrylic acid and itaconic acid, a copolymer of methacrylic acid and itaconic acid, a copolymer of maleic acid and styrene, a copolymer of acrylic acid and methacrylic acid, a copolymer of acrylic acid and methyl acrylate, a copolymer of acrylic acid and vinyl acetate, a copolymer of acrylic acid and maleic acid, N-methyl-fatty acid (C12-18) sarcosinate, carboxylic acids such as resin acid and fatty acid (C12-18) and the like, and salts of these carboxylic acids.

(B-2) Sulfate ester type surfactants: examples sulfate ester type surfactants include alkyl (C12-18) sulfate ester, polyoxyethylene alkyl (C12-18) ether sulfate ester, polyoxyethylene (mono or di) alkyl (C8-12) phenyl ether sulfate ester, sulfate ester of a polyoxyethylene (mono or di) alkyl (C8-12) phenyl ether polymer, polyoxyethylene (mono, di, or tri) phenyl phenyl ether sulfate ester, polyoxyethylene (mono, di, or tri) benzyl phenyl ether sulfate ester, polyoxyethylene (mono, di, or tri) styryl phenyl ether sulfate ester, sulfate ester of a polyoxyethylene (mono, di, or tri) styryl phenyl ether polymer, sulfate ester of a polyoxyethylene polyoxypropylene block polymer, sulfated oil, sulfated fatty acid ester, sulfated fatty acid, sulfate ester of sulfated olefin and the like, and salts of these sulfate esters.

(B-3) Sulfonic acid type surfactants: examples of sulfonic acid type surfactants include paraffin (C12-22) sulfonic acid, alkyl (C8-12) benzene sulfonic acid, formaldehyde condensation product of alkyl (C8-12) benzene sulfonic acid, formaldehyde condensation product of cresol sulfonic acid, -olefin (C14-16) sulfonic acid, dialkyl (C8-12) sulfosuccinic acid, lignin sulfonic acid, polyoxyethylene (mono or di) alkyl (C8-12) phenyl ether sulfonic acid, polyoxyethylene alkyl (C12-18) ether sulfosuccinate half ester, naphthalene sulfonic acid, (mono, or di) alkyl (C1-6) naphthalene sulfonic acid, formaldehyde condensation product of naphthalene sulfonic acid, formaldehyde condensation product of (mono, or di) alkyl (C1-6) naphthalene sulfonic acid, formaldehyde condensation product of creosote oil sulfonic acid, alkyl (C8-12) diphenyl ether disulfonic acid, Igepon T (tradename), polystyrene sulfonic acid, sulfonic acids of a styrene sulfonic acid-methacrylic acid copolymer and the like, and salts of these sulfonic acids.

(B-4) Phosphate ester type surfactants: examples of phosphate ester type surfactants include alkyl (C8-12) phosphate ester, polyoxyethylene alkyl (C12-18) ether phosphate ester, polyoxyethylene (mono or di) alkyl (C8-12) phenyl ether phosphate ester, phosphate ester of a polyoxyethylene (mono, di, or tri) alkyl (C8-12) phenyl ether polymer, polyoxyethylene (mono, di, or tri) phenyl phenyl ether phosphate ester, polyoxyethylene (mono, di, or tri) benzyl phenyl ether phosphate ester, polyoxyethylene (mono, di, or tri) styryl phenyl ether phosphate ester, phosphate ester of a polyoxyethylene (mono, di, or tri) styryl phenyl ether polymer, phosphate ester of a polyoxyethylene polyoxypropylene block polymer, phosphatidyl choline, phosphate ester of phosphatidyl ethanolimine and condensed phosphoric acid (for example, such as tripolyphosphoric acid) and the like, and salts of these phosphate esters. Salts of above-mentioned (B-1) to (B-4) include alkaline metals (such as lithium, sodium and potassium), alkaline earth metals (such as calcium and magnesium), ammonium and various types of amines (such as alkyl amines, cycloalkyl amines and alkanol amines).

(C) Amphoteric surfactants: Examples of amphoteric surfactants include betaine type surfactants and amino acid type surfactants.

The above surfactants may be used singly or in combination of two or more surfactants. Notably, organo-silicone surfactants may be combined with other surfactants. The total concentration of surfactants in the aqueous suspension of the invention may be easily tested by conducting comparative spraying experiments, similarly as done in the examples. However, in general, the total concentration of surfactants may be between 0.005 and 2 volume-%, between 0.01 and 0.5 volume-%, between 0.025 and 0.2 volume-% of the composition for application to the plant or plants. Since the density of surfactants is generally close to 1.0 g/ml, the total concentration of surfactants may be defined as being between 0.05 and 20 g per liter of the composition for application, between 0.1 and 5.0 g, or between 0.25 and 2.0 g per liter of the composition for application to the plants.

Cuticle Penetrating Agents

The methods and compositions of the present invention can include one or more cuticle penetrating agents, in order to penetrate waxy cuticle (or bark) of some plants and plant structures and facilitate access of the complexed polynucleotide-cell wall degrading enzyme to the cell wall of target plant cells.

As used herein, the term “cuticle penetrating agent” refers to any composition or compound which can weaken, permeabilize, ablate or otherwise alter a plant cuticle to allow penetration of the otherwise excluded or partially excluded compounds or compositions.

The plant cuticle consists of lipid and hydrocarbon polymers impregnated with wax, and is synthesized exclusively by the epidermal cells. The cuticle is composed of an insoluble cuticular membrane impregnated by and covered with soluble waxes. Cutin (a cross-linked polyester polymer) is the best-known structural component of the cuticular membrane. The cuticle can also contain the non-saponifiable hydrocarbon polymer cutan. Cuticle penetrating agents can be broadly classified into oils, fatty acids, waxes, soaps and grease, which may penetrate the cuticle through chemical interaction with cuticular waxy components, and abrasives, which can penetrate the cuticle by mechanically disrupting the waxy layers of the cuticle.

Care must be taken, though, in choosing a cuticle penetrating agent, as gaining access through the cuticle must be balanced with the extent of wounding the cuticle, exposing the softer tissue of the plant to the drying and physically erosive effects of the ambient environment. One abrasive suitable for use in the invention comprises a particulate material that is essentially insoluble in aqueous medium. The abrasive is believed to weaken, (notably if used together with a wetting agent), the surface of plant tissue such as leaves, and thereby facilitates penetration of the polynucleotide-cell wall degrading enzyme complex into the intercellular space of plant tissue, increasing the efficiency of transport of the polynucleotide into the plant cell.

The particulate material to be used as the abrasive of the invention may be carrier material as commonly used as carriers in wettable powder (WP) of pesticide formulations. In the context of wettable powders, these carriers are also referred to in the field of pesticide formulations as “fillers” or “inert fillers”. Wettable powder formulations are part of the general knowledge in the field of plant protection. Reference is made to the handbook PESTICIDE SPECIFICATIONS, “Manual for Development and Use of FAO and WHO Specifications for Pesticides”, edited by the World Health Organisation (WHO) and the FOOD and Agriculture Organization of the United States, Rome, 2002, ISBN 92-5-104857-6. Wettable powder formulations for plant protection are for example described in EP 1810569, EP1488697, EP1908348 and EP0789510. The abrasive may be a mineral material, typically an inorganic material. Examples of such carrier materials are diatomaceous earth, talc, clay, calcium carbonate, bentonite, acid clay, attapulgite, zeolite, sericite, sepiolite or calcium silicate. It is also possible to use quartz powder such as the highly pure quartz powder described in WO02/087324. Examplary abrasives are silica, such as precipitated and fumed hydrophilic silica, and carborundum, sand (silica oxide), pumice, aluminium oxide, silicon carbide and tungsten carbide.

The abrasive properties of diluents or fillers such as silica used in wettable powders are known (see “Pesticide Application Methods” by G. A. Matthews, third edition, Blackwell Science, 2000, on page 52 thereof).

As commercial products of particulate inorganic materials for use as abrasives in the invention, the hydrophilic silica Sipernat™ 22S and Sipernat™ 50 S, manufactured by Evonic Degussa may be mentioned. Other products are “Hi-Sil™ 257”, a synthetic, amorphous, hydrated silica produced by PPG Industries Taiwan Ltd. or “Hubersorb 600 ™”, a synthetic calcium silicate, manufactured by Huber Corporation. A commercial sub-micron sized silica is Hi-Sil™ 233 (PPG Industries) having an average particle size of around 0.02 μm.

The abrasive may have a median particle size between 0.01 and 40, between 0.015 and 30, between 0.05 and 30, between 0.1 and 30, between 0.1 and 20, between 0.5 and 20, and between 1.0 and 16 μm. In one embodiment, the median particle size is between 0.015 and 1 or between 0.02 and 0.5 μm. The median particle size is the volume median particle size that can be measured by laser diffraction using a Mastersizer™ from Malvern Instruments, Ltd. When the abrasive is applied by spraying, in order to avoid clogging of spraying nozzles, the maximum particle size of the largest particles contained in the abrasive should be at most 45 μm, or at most 40 μm, which may be determined by sieving. Typically, the particle sizes above relate to primary particle sizes.

The content of the abrasive in the composition of the invention may be between 0.01 and 3, between 0.02 and 2, between 0.05 and 1 and between 0.1 and 0.5% by weight of the composition for application onto the plant.

The cuticle penetrating agent can be an oil. Oils suitable for use as cuticular penetrating agents in the methods and compositions of the invention can be any oils which are tolerated by plants, e.g. are found non-toxic to the plant, and which facilitate penetration of the cuticle. Currently in common use for agricultural and horticultural application are a variety of plant-based oils, and narrow range petroleum spray oils (narrow range oil), also known as horticultural mineral oils. Most commonly mineral or petroleum spray oils are oils with ≥92% unsulfonated residues and distillation ranges at reduced pressure of ≤44 degrees centigrade between the 10% and 90% distillation points. (These oils were once commonly referred to as 60 s SUS viscosity petroleum spray oils, and are now generally equivalent to nC21 horticultural mineral oils). Less commonly, but also suitable are oils with 50% distillation points equal to 224 degrees C.±5 degrees and 10% to 90% distillation ranges≤52.8 degrees C. (once commonly referred to as 70 s SUS viscosity petroleum spray oils, now generally either nC23 horticultural or agricultural mineral oils).

Table 2 details a non-limiting list of commercially available oil and oil combinations used in agriculture/horticulture, suitable for use as cuticle penetrating agents in the compositions and methods of the present invention.

TABLE 2
Oils and Oil combinations for cuticle penetrating agents

| | PRINCIPAL | | | | |
|---|---|---|---|---|---|
| | FUNCTIONING | | | | MANUFACTURER/ |
| CATEGORY | AGENTS | USE RANGE | COMMENTS | PRODUCT NAME | DISTRIBUTOR |
| Nonionic Surfactant | Proprietary blend of | 0.25-1% | For aerial use | AERO DYNE-AMIC | Helena Chemical Co. |
| and | ethoxylated alkyl | | only, pH reduction | | |
| Methylated or | phosphate esters, | | and buffering, | | |
| Ethylated Vegetable | polyalkylene modified | | NIS and oil blend | | |
| Oil | polydimethylsiloxane, | | | | |
| and | nonionic emulsifiers and | | | | |
| Buffering Agent or | methylated vegetable oils | | | | |
| Acidifier | | | | | |
| Methylated or | Methylated seed oil, | 0.125-0.5% | *CPDA certified, | AIRFORCE | United Suppliers, Inc. |
| Ethylated Vegetable | organosilicones, and | | wetter, canopy | | |
| Oil | nonionic surfactant | | penetration | | |
| Methylated or | Methylated vegetable oil | 1-2 pt/A | | BRANDT MSO | Brandt Consolidated, |
| Ethylated Vegetable | and surfactant blend | | | | Inc. |
| Oil | | | | | |
| Organo-Silicone | Proprietary blend of | 0.125-0.75% | | CHEMPRO S-172 | Chemorse, Ltd. |
| Surfactant | polyalkyleneoxide modified | | | | |
| and | polydimethylsiloxane, | | | | |
| Methylated or | nonionic emulsifiers and | | | | |
| Ethylated Vegetable | methylated seed oil | | | | |
| Oil | | | | | |
| Methylated or | Methylated canola oil, | see label | | CM2 CAN-HANCE | Brandt Consolidated, |
| Ethylated Vegetable | polyalkyleneoxide | | | | Inc. |
| Oil | heptamethyltrisiloxane, | | | | |
| | alkylphenol ethoxylate | | | | |
| Methylated or | Ethyl oleate, sorbitan | 1-4 pt/A | *CPDA certified, | COMPETITOR | Wilbur-Ellis Company |
| Ethylated Vegetable | alkylpolyethoxylate ester, | | modified | | |
| Oil | dialkyl polyoxyethylene | | vegetable oil | | |
| | glycol | | | | |
| Methylated or | Methylated vegetable oil | 1-2 pt/A | | CONQUER | Chemorse, Ltd. |
| Ethylated Vegetable | and surfactant blend | | | | |
| Oil | | | | | |
| Methylated or | Alkaline buffered | 1% | Increases water | CORNBELT BASE | Van Diest Supply Co. |
| Ethylated Vegetable | methylated seed oil, | | pH | | |
| Oil | nitrogen-based fertilizer | | | | |
| and | solution, nonionic | | | | |
| Basic Blend | surfactant and antifoam | | | | |
| | agent | | | | |
| Methylated or | Blend of methylated | 1.5-2 pt/A | Contains antifoam | CORNBELT | Van Diest Supply Co. |
| Ethylated Vegetable | soybean oil and surfactant | | | METHYLATED SOY- | |
| Oil | emulsifier | | | STIK | |
| Nonionic Surfactant | Fatty acid complex | 0.25% | 95% active low- | CORNBELT TROPHY | Van Diest Supply Co. |
| and | alkoxylate, free fatty | | foaming | GOLD | |
| Methylated or | acids, and | | replacement for | | |
| Ethylated Vegetable | alkylarylalkoxylate | | COCs and MSOs | | |
| Oil | | | | | |
| Methylated or | Cottonseed oil and | 1-8 qt/A | | COTTONSEED PLUS | Wilbur-Ellis Company |
| Ethylated Vegetable | emulsifiers | | | | |
| Oil | | | | | |
| Methylated or | Methylated soybean oil | 0.125-1% | Discontinued | DESTINY | Winfield Solutions, |
| Ethylated Vegetable | plus emulsifiers | | product | | LLC |
| Oil | | | | | |
| Methylated or | Methylated seed and | 0.5-1% | Deposition agent | DLZ | Helena Chemical Co. |
| Ethylated Vegetable | paraffin oil, nonionic | | | | |
| Oil | surfactants | | | | |
| and | | | | | |
| Other | | | | | |
| Nonionic Surfactant | Proprietary blend of | 0.375-0.75% | Blend of | DYNE-AMIC | Helena Chemical Co. |
| and | polyethoxlated dimethyl | | organosilicone | | |
| Organo-Silicone | siloxanes, alkylaryl | | surfactants and | | |
| Surfactant | ethoxylates and | | methylated seed | | |
| and | methylated seed oils | | oils | | |
| Methylated or | | | | | |
| Ethylated Vegetable | | | | | |
| Oil | | | | | |
| Methylated or | Modified vegetable oil, | 0.25-1% | | ELITE SUPREME | Red River Specialties, |
| Ethylated Vegetable | blend of organosilicone | | | | Inc. |
| Oil | and nonionic emulsifier | | | | |
| | 84-16 | | | | |
| Methylated or | Methylated seed oil, | 1-2.5% | Buffers pH to | ENTRO LIQUID | KALO, Inc. |
| Ethylated Vegetable | nitrogen based fertilizer | | minimize acid | | |
| Oil | solution, surfactant blend, | | hydrolysis, | | |
| and | alkali buffer and antifoam | | retention aid | | |
| Basic Blend | | | | | |
| Methylated or | Poly (methylene-p-nonyl | 0.5-1% | Nonionic | EXIT | Miller Chemical Co. |
| Ethylated Vegetable | phenoxy) poly | | activator- | | |
| Oil | (oxypropylene) propanol, | | enhancer | | |
| | modified resin, esterfied | | | | |
| | oil | | | | |
| Methylated or | Soybean oil, methyl ester, | 1-2 pt/A | Higher rates on | EXURO | United Suppliers, Inc. |
| Ethylated Vegetable | polymeric nonionic and | | hard-to-control/ | | |
| Oil | alkyl and aryl ethoxylate | | stressed weeds | | |
| | blend | | | | |
| Organo-Silicone | Proprietary blend of | 0.75-2% | Blend of refined | FASTSTRIKE | J.R. Simplot |
| Surfactant | polyalkyleneoxide modified | | and modified | | Company |
| and | polydimethylsiloxane | | spray oil and | | |
| Methylated or | nonionic emulsifiers and | | nonionic | | |
| Ethylated Vegetable | methylated vegetable oil | | organosilicone | | |
| Oil | | | | | |
| Methylated or | Methylated seed oil, | 1 pt/A | | FIRE ZONE | Helena Chemical Co. |
| Ethylated Vegetable | polyethoxylated aliphatics, | | | | |
| Oil | phosphorated | | | | |
| | polyethoxlated alkanoates | | | | |
| Nonionic Surfactant | Lecithin, methylesters of | see label | Penetrant, wetter | FRANCHISE | Loveland Products, |
| and | fatty acids and alcohol | | | | Inc. |
| Methylated or | ethoxylate | | | | |
| Ethylated Vegetable | | | | | |
| Oil | | | | | |
| Methylated or | Methylated soybean oil | 20-24 oz/A | | FS MSO ULTRA | GROWMARK, Inc. |
| Ethylated Vegetable | and surfactants | | | | |
| Oil | | | | | |
| Methylated or | Methylated canola oil and | 20-24 oz/A | *CPDA certified | FS OPTIQUE | GROWMARK, Inc. |
| Ethylated Vegetable | surfactants | | | | |
| Oil | | | | | |
| Methylated or | Ethyl oleate, polyethylene | see label | *CPDA certified | HASTEN | Wilbur-Ellis Company |
| Ethylated Vegetable | dialky ester, ethoxylated | | | | |
| Oil | nonylphenol | | | | |
| Methylated or | Methylated soybean oil | 1-2 pt/A | Easily dispersed | HOME RUN | Conklin Co., Inc. |
| Ethylated Vegetable | plus emulsifier package | | for trouble-free | | |
| Oil | | | mixing | | |
| Organo-Silicone | Proprietary blend of | 0.25-0.63% | Blend of MSO and | INERGY | Winfield Solutions, |
| Surfactant | modified vegetable oil, | | organo silicone | | LLC |
| and | polyalkyleneoxide modified | | | | |
| Methylated or | dimethylpolysiloxane and | | | | |
| Ethylated Vegetable | nonionic emulsifiers | | | | |
| Oil | | | | | |
| Methylated or | Methyl esters of fatty | 1.5-2 pt/A or | | INVADE | Innvictis Crop Care, |
| Ethylated Vegetable | acids, nonylphenol | 1% | | | LLC |
| Oil | ethoxylate | | | | |
| Organo-Silicone | Methyl esters of fatty | 4-32 oz/A | | INVADE RST | Innvictis Crop Care, |
| Surfactant | acids, nonylphenol | | | | LLC |
| and | ethoxylate and | | | | |
| Methylated or | ethoxylated | | | | |
| Ethylated Vegetable | heptamethyltrisiloxane | | | | |
| Oil | | | | | |
| Crop Oil | D-Limonene and natural | 0.125-0.5% | Odor masking | KAMMO PLUS | Helena Chemical Co. |
| (Petroleum) | plant oils | | | | |
| Concentrate | | | | | |
| and | | | | | |
| Methylated or | | | | | |
| Ethylated Vegetable | | | | | |
| Oil | | | | | |
| Nonionic Surfactant | Lecithin, methyl esters of | 0.125-1% | Penetrant, | LIBERATE | Loveland Products, |
| and | fatty acids, and alcohol | | deposition, drift | | Inc. |
| Methylated or | ethoxylate | | reduction | | |
| Ethylated Vegetable | | | | | |
| Oil | | | | | |
| and | | | | | |
| Deposition (Drift | | | | | |
| Control) and/or | | | | | |
| Retention Agent | | | | | |
| Methylated or | Methylated seed oil plus | 1.5-2 pt/A | | MES-100 | Drexel Chemical Co. |
| Ethylated Vegetable | emulsifiers | | | | |
| Oil | | | | | |
| Methylated or | Methylated canola oil plus | 0.5-1% | | METH-N-OIL | Jay-Mar, Inc. |
| Ethylated Vegetable | surfactants | | | | |
| Oil | | | | | |
| Methylated or | Methylated seed oil | 1.5-2 pt/A | | METHSOYOIL | AgXplore |
| Ethylated Vegetable | | | | | International, Inc. |
| Oil | | | | | |
| Methylated or | Methylated soybean oil | 1.5-2 pt/A | | METHYLATED | UCPA LLC |
| Ethylated Vegetable | and surfactant blend | | | SOYBEAN OIL PLUS | |
| Oil | | | | | |
| Methylated or | Methylated soyoil plus | 1.5-2 pt/A | Contains antifoam | MISSION | Southern States |
| Ethylated Vegetable | surfactants and emulsifiers | | | | Cooperative, Inc. |
| Oil | 90-10 | | | | |
| Methylated or | Modified vegetable oil, | 1.5-2 pt/A | Methylated | MODIFIED | KALO, Inc. |
| Ethylated Vegetable | alkylphenol ethoxylate, | | soybean oil with | VEGETABLE OIL | |
| Oil | polysiloxane | | emulsifier | | |
| Methylated or | Modified seed oil, alkyl | 0.5-2 pt/A | | MONTEREY MSO | Brandt Consolidated, |
| Ethylated Vegetable | phenol ethoxylate and free | | | | Inc. |
| Oil | fatty acids | | | | |
| Methylated or | Proprietary blend of | 1-2 pt/A | | MSO | Helena Chemical Co. |
| Ethylated Vegetable | methylated oils and | | | | |
| Oil | nonionic surfactant | | | | |
| and | | | | | |
| Methylated or | | | | | |
| Ethylated Vegetable | | | | | |
| Oil | | | | | |
| Methylated or | Methylated seed oils plus | 1-2 pt/A | *CPDA certified | MSO | Loveland Products, |
| Ethylated Vegetable | emulsifying surfactants | | | CONCENTRATE | Inc. |
| Oil | | | | WITH LECITECH | |
| Methylated or | Proprietary blend of | 1-2 pt/A or | 80% MSO with | MSO ULTRA | Precision Labs, Inc. |
| Ethylated Vegetable | methyl soyate, nonionic | 1% | 20% surfactant | | |
| Oil | surfactants and emulsifiers | | | | |
| | 80-20 | | | | |
| Methylated or | Methylated soyoil plus | 1-2 pt/A | Contains | NOBLE | Winfield Solutions, |
| Ethylated Vegetable | surfactants/emulsifiers | | surfactant | | LLC |
| Oil | (90:10) | | | | |
| High Surfactant Oil | Methylated vegetable oil, | 0.375-0.63% | Blend of modified | ORGANOSILICONE | KALO, Inc. |
| Concentrate | polyether modified | | spray oil and | MVO | |
| and | polysiloxane, alkylphenol | | organosilicone | | |
| Methylated or | ethoxylate | | surfactant | | |
| Ethylated Vegetable | | | | | |
| Oil | | | | | |
| Methylated or | 85-15 methylated canolate | 1-2 pt/A or | *CPDA certified | PERSIST ULTRA | Precision Labs, Inc. |
| Ethylated Vegetable | | 1% | | | |
| Oil | | | | | |
| Methylated or | Methylated vegetable oil, | 1.5-2 pt/A | Methylated canola | PERSIST ULTRA | J.R. Simplot |
| Ethylated Vegetable | alkylphenol ethoxylates | | seed oil | | Company |
| Oil | | | | | |
| Organo-Silicone | Methylated seed oil plus | 0.125-0.5% | none | PHASE | Loveland Products, |
| Surfactant | organosilicone surfactant | | | | Inc. |
| and | | | | | |
| Methylated or | | | | | |
| Ethylated Vegetable | | | | | |
| Oil | | | | | |
| Methylated or | Carbamides, alcohol | 1% | Improves uptake, | PHASE II | Loveland Products, |
| Ethylated Vegetable | ethoxylates, methyl esters | | spreading, | | Inc. |
| Oil | and polyether modified | | coverage, | | |
| | polysiloxane | | penetration | | |
| Methylated or | Methylated soybean oil | 1.5-2 pt/A | none | PIERCE | J.R. Simplot |
| Ethylated Vegetable | with selected emulsifiers | | | | Company |
| Oil | | | | | |
| Methylated or | Methylated soybean oil, | 1-2 pt/A | Soy based, | PREMIUM MSO | Garrco Products, Inc. |
| Ethylated Vegetable | emulsifer blend | | narrow range | | |
| Oil | | | methyl ester | | |
| Nonionic Surfactant | Modified vegetable oil, | 1-2.5% | Unique blend, | RENEGADE | Wilbur-Ellis Company |
| and | ammonium solution, | | high load of N | | |
| Methylated or | nonionic surfactant | | | | |
| Ethylated Vegetable | | | | | |
| Oil | | | | | |
| and | | | | | |
| Basic Blend | | | | | |
| Organo-Silicone | Methylated seed oil plus | 0.375-0.75% | none | RIVET | Winfield Solutions, |
| Surfactant | organosilicone surfactant | | | | LLC |
| and | | | | | |
| Methylated or | | | | | |
| Ethylated Vegetable | | | | | |
| Oil | | | | | |
| Methylated or | Proprietary blend of oils, | 0.5-1% | High or low | RRSI SUNRISE | Red River Specialties, |
| Ethylated Vegetable | emulsifiers, organosilicone | | volume | | Inc. |
| Oil | and formulation aids | | applications | | |
| Methylated or | Methylated seed oil blend | 0.75-12 qt/A | none | RRSI SUNSET | Red River Specialties, |
| Ethylated Vegetable | and emulsifiers | | | | Inc. |
| Oil | | | | | |
| Organo-Silicone | Blend of organosilicone, | 0.375-0.75% | 100% active, | SIL-MES 100 | Drexel Chemical Co. |
| Surfactant | methylated seed oil, | | wetter, spreader | | |
| and | alcohol ethoxylate and NIS | | | | |
| Methylated or | | | | | |
| Ethylated Vegetable | | | | | |
| Oil | | | | | |
| Methylated or | Methylated seed oils plus | 0.25-0.75% | Contains | SPECTRUM | Coastal |
| Ethylated Vegetable | nonionic surfactant | | organosilicone | | Agrobusiness, Inc. |
| Oil | | | surfactant | | |
| Methylated or | Methylated canola oil plus | 0.375-0.63% | none | SPIRIT | Jay-Mar, Inc. |
| Ethylated Vegetable | organosilicone surfactant | | | | |
| Oil | | | | | |
| Methylated or | Methylated soybean oil | 1-2 pt/A | *CPDA certified, | SUCCEED | United Suppliers, Inc. |
| Ethylated Vegetable | and surfactant blend | | methyl ester soy | | |
| Oil | | | oil, emulsifier | | |
| High Surfactant Oil | Soybean oil, methyl ester, | 0.25-0.75% | One half the rate | SUCCEED ULTRA | United Suppliers, Inc. |
| Concentrate | fatty acid, alkylphenol | | of conventional | | |
| and | ethoxylates, and alcohol | | methylated seed | | |
| Methylated or | ethoxylates | | oil concentrates | | |
| Ethylated Vegetable | | | | | |
| Oil | | | | | |
| Methylated or | Blend of methylated seed | 0.25-0.5% | Seed oil | SUN WET | Brewer International |
| Ethylated Vegetable | oil and emulsifiers | | surfactant | | |
| Oil | | | | | |
| Methylated or | Methylated soyoil plus | 1.5-2 pt/A | *CPDA certified, | SUNDANCE II | Rosen's, Inc. |
| Ethylated Vegetable | surfactants and emulsifiers | | contains antifoam | | |
| Oil | 90-10 | | | | |
| Methylated or | Methyl soyate UAN 28 | 40 oz/A | none | SUPER KIX | Wilbur-Ellis Company |
| Ethylated Vegetable | blend | | | | |
| Oil | | | | | |
| and | | | | | |
| Nitrogen Source | | | | | |
| Methylated or | Methyl soyate nonylphenol | 1-4 pt/A | *CPDA certified, | SUPER SPREAD | Wilbur-Ellis Company |
| Ethylated Vegetable | blend | | 100% active | MSO | |
| Oil | | | | | |
| Organo-Silicone | Organosilicone and | see label | *CPDA certified, | SYL-TAC | Wilbur-Ellis Company |
| Surfactant | modified vegetable seed | | | | |
| and | oil blend | | | | |
| Methylated or | | | | | |
| Ethylated Vegetable | | | | | |
| Oil | | | | | |
| Methylated or | Methylated seed oils, | 0.5%-1% | none | SYNERGY | Coastal |
| Ethylated Vegetable | surfactants and emulsifiers | | | | Agrobusiness, Inc. |
| Oil | | | | | |
| Organo-Silicone | Proprietary blend of | 0.25-0.63% | Blend of MSO and | TURBULENCE | Winfield Solutions, |
| Surfactant | modified vegetable oil, | | organo silicone | | LLC |
| and | polyalkyleneoxide modified | | | | |
| Methylated or | dimethylpolysiloxane and | | | | |
| Ethylated Vegetable | nonionic emulsifiers | | | | |
| Oil | | | | | |
| Methylated or | Proprietary blend of | 1% v/v | Compatibility | ZAAR | Helena Chemical Co. |
| Ethylated Vegetable | methylated oils and | | agent | | |
| Oil | nonionic surfactant | | | | |
| and | | | | | |
| Buffering Agent or | | | | | |
| Acidifier | | | | | |
| and | | | | | |
| Water Conditioning | | | | | |
| Agent | | | | | |

In a specific embodiment, refined mineral oil such as SK EnSpray99™ (SK Corp, Seoul Korea) is used as a cuticle penetrating agent. Oils suitable for use as cuticle penetrating agents can be provided in a range of concentrations, varying, for example, according to the type of plant and/or plant structure (leaf, stem, etc). In some embodiments, the refined mineral oil is provided in a sprayable form, in an aqueous carrier (e.g. phosphate buffer, water), at concentrations in the range of 0.05% to 5%, 0.1% to 3%, 0.5% to 2%, 0.75% to 2%, 1% to 1.5%, or 0.1, 0.2, 0.3, 0.4, 0.5, 0.6, 0.7, 0.8, 0.9, 1.0. 1.25, 1.5, 1.75, 2.0, 2.25, 2.5, 2.75, 3.0, 3.5, 4, 4.5, 5, 5.5 or 6.0% of the oil. In a specific embodiment, the oil is refined mineral oil and is provided to the plant at a concentration of about 1% weight/volume.

The compositions and methods of the invention can be used to deliver a polynucleotide to a plant cell having a cell wall. It will be appreciated that application of the compositions (contacting the plant cell) can be effected via a number of plant structures (e.g. leaves, stem, root) and in a number of different ways. Methods of application suitable for use with the compositions and methods of the invention include, but are not limited to spraying, dusting, soaking, injecting, aerosol application, particle bombardment, irrigation, positive or negative pressure application, girdling, ground deposition, trunk drilling and shoot drilling. Briefly, the methods of application can be divided into topical, irrigation and invasive.

Topical: Spraying, Dusting, Aerosol

Spraying—a way of covering crop foliage with a fluid based medium (i.e. water) mixed with compositions of interest. The method is based on producing high pressure within the tank and release of this pressure through the specialised spray equipment is what assists in covering the total plant foliage with the water and its contents. Spraying can be done from the ground manually with hand held back pack sprayers or with high pressure air-blast spraying equipment either pulled by tractors or self propelled or from the air with aircraft equipped with the necessary equipment to spray fields or orchards from above.

Aerosol application—similar to spraying, however, the composition can be formed into an aerosol (fine particles) from a liquid or non-liquid (dry). Aerosol application can be delivered from a high pressurised can or similar container.

Dusting—a method of spraying crops with products in powder form either from the ground or from the air with specialised aircraft. Components of the compositions of the invention that can be delivered in dry (non-liquid) form can be provided by dusting.

Brushing—Fluid or semi-fluid compositions of interest can be applied topically, directly, by brushing onto the surface of the plant or plant structure.

Irrigation: Irrigation, Drenching (Soaking)

Irrigation—the artificial application of water to land or soil. Compositions which can be dissolved in liquid (water) or formed into suspensions can be provided by irrigation. Irrigation is suitable for agricultural crops, maintenance of landscapes and gardens. Common methods of irrigation include flood, sprinkler and drip irrigation.

Drenching—a specific method of irrigation whereby the product of interest which is to be applied to the plant is mixed in a small amount of water which is applied around and in immediate proximity to the plant and its root systems.

Similar to Irrigation:

Ground deposition—the application of a composition for plants via the soil but not directly through irrigation or watering. The solid or liquid composition is inserted manually just under or on the surface of the top soil and then taken up by the plant roots when they are activated or incorporated into the soil by active irrigation or rain.

Invasive: Injection, Particle Bombardment, Girdling, Drilling

Injecting—an infusion method based on application of a fluid comprising a composition of interest into the upper foliage of a crop plant—trunk, stem, petiole, branches, leaves, etc usually with syringe, or similar pump equipment designed to create high pressure at a single entry point through a hollow needle or similar, which is inserted/pierced through the outer cuticle, bark, membrane, etc of the plant to a sufficient depth for the contents to be administered into the plant.

Particle bombardment—is commonly used method for genetic transformation of plants and other organisms. It is also known as biolistics and is the process by which large numbers of metal particles coated with a composition of interest (polynucleotide, dsRNA, etc) are shot at cells or plant tissue using a biolistic device or “gene gun”. It allows or enables cell wall penetration in order to assist in transferring large molecules (e.g. polynucleotides) of interest into plant cells.

Girdling—the complete removal of a strip of bark (consisting of cork cambium, phloem, cambium and sometimes going into the xylem) from around the entire circumference of either a branch or trunk of a woody plant, and application of the composition of interest directly on the de-barked area. In some cases only the layer just under the bark can be removed for application purposes (in order to minimize damage to the tree).

Trunk and shoot drilling—the insertion of a composition of interest directly into the tree trunk or shoot by directly physically drilling a hole in the trunk or shoot and applying the composition of interest (e.g. dsRNA-peptide-CWDE) through this hole either using gravity or by a pressure pump—either manually or mechanically. For young green shoots a metal needle and syringe can be used to produce the hole and can then be inserted into the hole for delivery.

In some embodiments, the plant cell is contacted with the polynucleotide and CWDE, or other compositions of the invention by topical application. In one embodiment, the plant is prepared for topical application (e.g. spraying, dusting or brushing) of the composition by abrasive treatment of the plant surface, to remove or partially remove the cuticle or bark and expose plant cell walls to the action of the CWDE. Abrasive spray can be delivered by an airbrush, for example, with high accuracy and safety to the plant. In other embodiments, the plant surface is first exposed by spraying of oil or surfactant. The inventors have found that, for tomato and citrus plants, for example, spraying of mineral oil, at about 1% w/v, is well tolerated by the plants and provides access for the polynucleotide and CWDE, or other compositions of the invention to the plant cells. In some embodiments, the oil is spraying on to the plant(s), until run off, the plants washed with water and then dried.

Spraying of oil or abrasives, in preparation for application of the compositions of the invention can be performed using any device providing a pressurized compartment for the sprayed material, connected to a spray nozzle (e.g. full cone, hollow cone, fan type nozzles). Spraying pressure can be in the range of 1-100 PSI, 5-80 PSI, 10-50 PSI, 15-45 PSI, 20-30 PSI, specifically about 1, about 2, about 3, about 4, about 5, about 6, about 7, about 8, about 9, about 10, about 12, about 15, about 18, about 20, about 23, about 28, about 30, about 35, about 40, about 45, about 50 PSI or more. In some embodiments, when spraying oils, the pressure can be in the range of 1-15, 3-12 or 5-10 PSI. In specific embodiments, the pressure for spraying oils (e.g. mineral oil) is 5-10 PSI. In some embodiments, when spraying abrasives (e.g. carborundum), the pressure can be in the range of 5-25, 10-30 or 15-50 PSI. In specific embodiments, the pressure for spraying abrasives is about 40 PSI. It will be appreciated that individual pressure and duration of spraying can vary with the type of plant, stage of growth, plant structure targeted, type of sprayed material, type of spray nozzle, weather conditions, etc.

Duration of spraying suitable for use with the compositions and methods of the invention can be in the range of 0.1-10 seconds, 0.5-5 seconds, 1.0-5 seconds, 2-4 seconds, about 1-1.5 seconds, specifically about 0.1, about 0.2, about 0.3, about 0.4, about 0.5, about 0.6, about 0.7, about 0.8, about 0.9, about 1.0, about 1.2, about 1.5, about 1.8, about 2.0, about 2.3, about 2.8, about 3.0, about 3.5, about 4.0, about 4.5, about 5.0 seconds or more. In some embodiments, the spraying can be 1-1.5 seconds. It will be appreciated that spraying large areas of crops can be achieved by mechanized equipment, such as tractor-powered sprayers, or aerial spray equipment (especially for spraying oil), and that spray duration will depend on speed of the sprayer and width of spray “cone”. Manufacturers specifications regarding distance from plant and pressure can provide guidelines for determination of spray pressure and duration.

It will be appreciated that the oils and/or abrasives can be applied separately, i.e. prior to application of polynucleotides and CWDE, or other compositions of interest. Following the exposure of the plant surface by abrasives, surfactants or oils, the polynucleotides and CWDE, or other compositions of interest can then be topically applied, by spraying, aerosol, dusting and/or brushing onto the plant (e.g. leaves) surface. In other embodiments, the oils and/or abrasives can be sprayed onto the plants along with polynucleotides and CWDE, or other compositions of interest, for example, the oils and/or abrasives and polynucleotides and CWDE, or other compositions of interest formulated together for spraying in a single composition or formulation.

In some embodiments, the CWDE is mixed with the compositions of the invention briefly (i.e. no more than 5, 10, 15, 20, 30, 40, 50 minutes, one hour, two hours, three hours, five hours, six, seven eight, ten, twelve hours, up to one day) or days (no more than one day, two days, three days, four days, five days, six days, one week or ten days) before application to the plant surface.

In some embodiments, the plant cell is contacted with the polynucleotide and CWDE, or other compositions of the invention by irrigation. Due to the absence of bark or cuticle barriers in the underground portions of most plants, when applied by irrigation, methods for exposing the plant cells (abrasives, surfactant, oils) may be foregone, and the polynucleotide and CWDE, or other compositions of the invention can be provided directly to the plant.

When the contacting of the plant cell is effected via injection and girdling, methods for exposing the plant cells can be foregone, due to the direct application of the compositions below the strata of wax or bark.

Thus, in some embodiments, wherein the contacting is effected via spraying, dusting, aerosol application or particle bombardment, the method of the invention comprises contacting a plant or organ thereof comprising the plant cell with the surfactant or cuticle penetrating agent or both, and subsequently contacting the plant or organ thereof with the polynucleotide and the cell wall degrading enzyme and at least one of a nucleic acid, a condensing agent, a transfection reagent and a surfactant, thereby delivering the polynucleotide to the plant cell.

In embodiments wherein the contacting is effected via injection, the method of the invention comprising injecting a plant or organ thereof comprising the plant cell with the polynucleotide and the cell wall degrading enzyme and at least one of a nucleic acid condensing agent, a transfection reagent and a surfactant thereby delivering the polynucleotide to the plant cell. It will be appreciated that in some cases, when delivered by injection, or via girdling, or in some cases even by topical application, the compositions of the invention can be delivered to plant tissues providing direct access to cell contents, for example, within cells comprising the sieve tubes, which allow rapid dissemination of the composition and dsRNA (or RNAi products thereof) of the invention.

In embodiments wherein the contacting is effected via irrigation, the method of the invention comprises contacting a plant or organ thereof comprising the plant cell with the polynucleotide and the cell wall degrading enzyme and at least one of a nucleic acid condensing agent, a transfection reagent and a surfactant, thereby delivering the polynucleotide to the plant cell. In embodiments wherein the contacting is via irrigation, the composition of the invention can be formulated for irrigation, i.e. as a fluid which is easily applied and taken up by the soil, for example, as an aqueous formulation, with or without agriculturally acceptable fluid carrier Wherein the contacting is dusting or aerosol, the composition may also be formulated as a dry powder or solid, with or without agriculturally acceptable carriers and/or fillers, excipients and the like. Wherein the contacting is by topical application, such as brushing, or by injection, the composition may be formulated as a fluid, as a dry powder or solid, or as a gel, with or without agriculturally acceptable carriers.

Thus, in some embodiments, the composition of the invention comprises a polynucleotide, a cell wall degrading enzyme and a nucleic acid condensing agent, or a polynucleotide, a cell wall degrading enzyme and a transfection reagent, or a polynucleotide, a cell wall degrading enzyme and a surfactant, or a polynucleotide, a cell wall degrading enzyme and a cuticle penetrating agent. According to some embodiments, the composition comprises a polynucleotide, a cell wall degrading enzyme and any combination of two or more of a nucleic acid condensing agent, a transfection reagent, a surfactant, and a cuticle penetrating agent. In some embodiments (for example, for irrigation and/or injection), the composition may be absent the cuticle penetrating agent.

It will be noted that in some cases, RNA interference has been shown to spread throughout a plant in response to local application of dsRNA. Thus, beneficial effects of the presence and action of dsRNA delivered to plant cells by the methods and compositions of the present invention can be afforded to remote organs and structures of the plant, for example, delivery of dsRNA to roots by irrigation may provide RNAi products (siRNA and miRNA) to stems, leaves, shoots and flowers of the plant.

The term “plant” as used herein encompasses whole plants, ancestors and progeny of the plants and plant parts, including leaves, flowers, fruit, buds, seeds, bulbs, embryo, seed pod, shoots, stems, roots (including tubers), and isolated plant cells, tissues and organs. The plant may be in any form including suspension cultures, embryos, meristematic regions, callus tissue, leaves, gametophytes, sporophytes, pollen, and microspores.

As used herein the phrase “plant cell” refers to plant cells which are derived and isolated from disintegrated plant cell tissue or plant cell cultures.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present invention may comprise a particular type of a plant cell or a plurality of different types of plant cells. It should be noted that optionally plant cultures featuring a particular type of plant cell may be originally derived from a plurality of different types of such plant cells.

Any commercially or scientifically valuable plant is envisaged in accordance with these embodiments of the invention. Plants that are particularly useful in the methods of the invention include all plants which belong to the super family Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, eucalyptus, a tree, an ornamental plant, a perennial grass and a forage crop. Alternatively algae and other non-Viridiplantae can be used for the methods of the present invention.

According to some embodiments of the invention, the plant used by the method of the invention is a crop plant including, but not limited to, cotton, Brassica vegetables, oilseed rape, sesame, olive tree, palm oil, banana, wheat, corn or maize, barley, alfalfa, peanuts, sunflowers, rice, oats, sugarcane, soybean, turf grasses, barley, rye, sorghum, sugar cane, chicory, lettuce, tomato, zucchini, bell pepper, eggplant, cucumber, melon, watermelon, beans, hibiscus, okra, apple, rose, strawberry, chile, garlic, pea, lentil, canola, mums, arabidopsis, broccoli, cabbage, beet, quinoa, spinach, squash, onion, leek, tobacco, potato, sugarbeet, papaya, pineapple, mango, Arabidopsis thaliana, and also plants used in horticulture, floriculture or forestry, such as, but not limited to, poplar, fir, eucalyptus, pine, an ornamental plant, a perennial grass and a forage crop, coniferous plants, moss, algae, as well as other plants listed in World Wide Web (dot) nationmaster (dot) com/encyclopedia/Plantae.

According to a specific embodiment of the present invention, the plant comprises tomato plants. In some embodiments, the tomato plant is Tiny Tim tomato.

“Zebra chip” (or “papa manchada” or “papa rayada”) is a disease in potatoes caused by Candidatus Liberibacter solanacearum, vectored by the potato psyllid, which causes discoloration and impaired flavor of the potato when fried. Potato crops worldwide are now endangered by the rapid spread of this bacterial disease. Delivery of dsRNA, targeting the pathogen itself, the vector or components of the potato's response mechanisms, to potato crops, within the context of the methods and compositions of the present invention, may provide effective means for prevention and treatment to counter the growing threat to this important branch of world agriculture. Any method of application of the compositions of the invention is suitable for potato plants, but as potato is a tuber, administration to the below ground structures, such as irrigation, drenching and the like, or to the above ground structures of the plant (e.g. leaves), such as spraying, dusting and the like, may be most advantageous in treating potato plants.

Thus, according to a specific embodiment, the plant cell or plant of the invention is a potato plant. In some embodiments, the potato plant is a diseased potato plant, for example, having had contact with Candidatus Liberibacter solanacearum. In other embodiments, the potato plant at risk of contact with C. Liberibacter solanacearum (LSO).

According to some embodiments, the plant used by the method of the invention is a crop plant.

According to a specific embodiment, the plant is selected from the group consisting of citrus plants, including, but not limited to all citrus species and subspecies, including sweet oranges commercial varieties (Citrus sinensis Osbeck (L.), clementines (C. reticulata), limes (C. aurantifolia), lemon (C. limron), sour orange (C. aurantium), hybrids and relatives (Citranges, Citrumelos, Citrandarins), Balsamocitrus dawei, C. maxima, C. jambhiri, Clausena indica, C. lansium, Triphasia trifolia, Swinglea glutinosa, Micromellum tephrocarpa, Merope spp., Eremolemon; Atalantia spp., Severinia buxifolia; Microcitrus spp., Fortunella spp., Calodendrum capense, Murraya spp. and Poncirus trifoliate. In some embodiments the citrus plant is an orange, a lemon, a lime, a grapefruit, a clementine, a tangerine or a pomello tree. The citrus tree can be a seed-grown tree or a grafted tree, grafted onto a different citrus rootstock.

According to some embodiments of the invention, delivering the polynucleotide to the plant cell increases at least one of yield, growth rate, vigor, biomass or stress tolerance of the plant. In some embodiments, the polynucleotide is delivered to the plant cell and can be expressed within the plant cell. Recombinant expression is effected by cloning a nucleic acid of interest (e.g., encoding a protein, an RNA of interest (dsRNA, RNAi) etc) into a nucleic acid expression construct under the translational control of a plant promoter.

Thus, there is provided a nucleic acid construct comprising a nucleic acid sequence of interest said nucleic acid sequence being under a transcriptional control of a regulatory sequence such as a plant tissue specific promoter.

A coding nucleic acid sequence is “operably linked” or “transcriptionally linked to a regulatory sequence (e.g., promoter)” if the regulatory sequence is capable of exerting a regulatory effect on the coding sequence linked thereto.

The term “regulatory sequence”, as used herein, means any DNA, that is involved in driving transcription and controlling (i.e., regulating) the timing and level of transcription of a given DNA sequence, such as a DNA coding for a miRNA or siRNA, precursor or inhibitor of same. For example, a 5′ regulatory region (or “promoter region”) is a DNA sequence located upstream (i.e., 5′) of a coding sequence and which comprises the promoter and the 5′-untranslated leader sequence. A 3′ regulatory region is a DNA sequence located downstream (i.e., 3′) of the coding sequence and which comprises suitable transcription termination (and/or regulation) signals, including one or more polyadenylation signals.

For the purpose of the invention, the promoter is a plant-expressible promoter. As used herein, the term “plant-expressible promoter” means a DNA sequence which is capable of controlling (initiating) transcription in a plant cell. This includes any promoter of plant origin, but also any promoter of non-plant origin which is capable of directing transcription in a plant cell, i.e., certain promoters of viral or bacterial origin. Thus, any suitable promoter sequence can be used by the nucleic acid construct of the present invention. According to some embodiments of the invention, the promoter is a constitutive promoter, a tissue-specific promoter or an inducible promoter (e.g. an abiotic stress-inducible promoter).

As used herein, the phrase “stress tolerance” refers to both tolerance to biotic stress, and tolerance to abiotic stress. The phrase “abiotic stress” as used herein refers to any adverse effect on metabolism, growth, viability and/or reproduction of a plant caused by a-biotic agents. Abiotic stress can be induced by any of suboptimal environmental growth conditions such as, for example, water deficit or drought, flooding, freezing, low or high temperature, strong winds, heavy metal toxicity, anaerobiosis, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present disclosure contemplates situations in which there is a single abiotic stress condition or alternatively situations in which two or more abiotic stresses occur.

As used herein the phrase “abiotic stress tolerance” refers to the ability of a plant to endure an abiotic stress without exhibiting substantial physiological or physical damage (e.g. alteration in metabolism, growth, viability and/or reproducibility of the plant).

According to some embodiments, delivering the polynucleotide to the plant cell using the methods and composition of the invention increases crop production. Crop production can be measured by biomass, vigor or yield, and can be used to calculate nitrogen use efficiency and fertilizer use efficiency. As used herein, the phrase “nitrogen use efficiency (NUE)” refers to a measure of crop production per unit of nitrogen fertilizer input. Fertilizer use efficiency (FUE) is a measure of NUE. The plant's nitrogen use efficiency is typically a result of an alteration in at least one of the uptake, spread, absorbance, accumulation, relocation (within the plant) and use of nitrogen absorbed by the plant. Improved crop production, vigor, yield, NUE or FUE is with respect to that of a plant lacking the polynucleotide of the invention of the same or similar species and developmental stage and grown under the same or similar conditions.

As used herein the term/phrase “biomass”, “biomass of a plant” or “plant biomass” refers to the amount (e.g., measured in grams of air-dry tissue) of a tissue produced from the plant in a growing season. An increase in plant biomass can be in the whole plant or in parts thereof such as aboveground (e.g. harvestable) parts, vegetative biomass, roots and/or seeds or contents thereof (e.g., oil, starch etc.).

As used herein the term/phrase “vigor”, “vigor of a plant” or “plant vigor” refers to the amount (e.g., measured by weight) of tissue produced by the plant in a given time. Increased vigor could determine or affect the plant yield or the yield per growing time or growing area. In addition, early vigor (e.g. seed and/or seedling) results in improved field stand.

As used herein the term/phrase “yield”, “yield of a plant” or “plant yield” refers to the amount (e.g., as determined by weight or size) or quantity (e.g., numbers) of tissues or organs produced per plant or per growing season. Increased yield of a plant can affect the economic benefit one can obtain from the plant in a certain growing area and/or growing time.

According to one embodiment, the yield is measured by cellulose content, oil content, starch content and the like.

According to another embodiment, the yield is measured by oil content.

According to another embodiment, the yield is measured by protein content.

According to another embodiment, the yield is measured by seed number, seed weight, flower number or flower weight, fruit number or fruit weight per plant or part thereof (e.g., kernel, bean).

A plant yield can be affected by various parameters including, but not limited to, plant biomass; plant vigor; plant growth rate; seed yield; seed or grain quantity; seed or grain quality; oil yield; content of oil, starch and/or protein in harvested organs (e.g., seeds or vegetative parts of the plant); flower development, number of flowers (e.g. florets) per panicle (e.g. expressed as a ratio of number of filled seeds over number of primary panicles); harvest index; number of plants grown per area; number and size of harvested organs per plant and per area; number of plants per growing area (e.g. density); number of harvested organs in field; total leaf area; carbon assimilation and carbon partitioning (e.g. the distribution/allocation of carbon within the plant); resistance to shade; resistance to lodging, number of harvestable organs (e.g. seeds, flowers), seeds per pod, weight per seed, flowers per plant; and modified architecture [such as increase stalk diameter, thickness or improvement of physical properties (e.g. elasticity)].

According to some embodiments of aspects of the invention, fruit quality and yield are increased by introduction into the plant of the polynucleotide. Fruit yield can be measured according to harvest index (see above), expressed as number and/or size of fruit per plant or per growing area, and/or according to the quality of the fruit—fruit quality can include, but is not limited to sugar content, appearance of the fruit, shelf life and/or suitability for transport of the fruit, ease of storage of the fruit, increase in commercial value, fruit weight, juice weight, juice weight/fruit weight, rind weight, TSS—total soluble solids (°Brix), seed quality, symmetry, dry weight, TA—titrable acidity. MI—maturity index, CI—Colour index, peel colour, nutraceutical properties, vitamin C-ascorbic acid-content, hesperidin content, total flavonoids content and the like.

Improved plant NUE is translated in the field into either harvesting similar quantities of yield, while deploying less fertilizer, or increased yields gained by implementing the same levels of fertilizer. Thus, improved NUE or FUE has a direct effect on plant yield in the field.

As used herein “biotic stress” refers stress that occurs as a result of damage done to plants by other living organisms, such as bacteria, viruses, fungi, parasites, beneficial and harmful insects, weeds, and cultivated or native plants. It will be appreciated that, in some embodiments, improving or increasing vigor or growth rate of a plant according some aspects of some methods of the invention contributes to the overall health and robustness of the plant, thereby conferring improved tolerance to biotic, as well as abiotic stress.

In some embodiments of the invention, delivery of the polynucleotide to the plant cells according to the methods of the invention results in: improved tolerance of abiotic stress (e.g., tolerance of water deficit or drought, heat, cold, non-optimal nutrient or salt levels, non-optimal light levels) or of biotic stress (e.g., crowding, allelopathy, or wounding); a modified primary metabolite (e.g., fatty acid, oil, amino acid, protein, sugar, or carbohydrate) composition; a modified secondary metabolite (e.g., alkaloids, terpenoids, polyketides, non-ribosomal peptides, and secondary metabolites of mixed biosynthetic origin) composition; a modified trace element (e.g., iron, zinc), carotenoid (e.g., beta-carotene, lycopene, lutein, zeaxanthin, or other carotenoids and xanthophylls), or vitamin (e.g., tocopherols) composition; improved yield (e.g., improved yield under non-stress conditions or improved yield under biotic or abiotic stress); improved ability to use nitrogen or other nutrients; modified agronomic characteristics (e.g., delayed ripening; delayed senescence; earlier or later maturity; improved shade tolerance; improved resistance to root or stalk lodging; improved resistance to “green snap” of stems; modified photoperiod response); modified growth or reproductive characteristics; improved harvest, storage, or processing quality (e.g., improved resistance to pests during storage, improved fruit harvest, fruit storage, or fruit processing quality (e.g., improved resistance to pests during storage, improved resistance to breakage, improved appeal to consumers); or any combination of these traits.

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in NUE, in tolerance to stress, in growth rate, in yield, in biomass, in fruit quality, in height, in flower number, in water uptake or in vigor of a plant, as compared to the same or similar plant not receiving the polynucleotide according to the methods and compositions of the invention.

As used herein the term “decreasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater decrease in disease signs such as DSI, starch accumulation and the like of a plant.

According to some embodiments of the invention, plant parameters are monitored in the treated plants following delivery of the polynucleotide. In some embodiments, parameters of plant health, vigor, etc are monitored, for example, expression of pathogen resistance response genes, parameters of the plant's tolerance to stress, growth rate, yield, biomass, fruit quality or vigor of the plant. In some embodiments, monitoring of the plant parameters (of gene expression and/or plant tolerance to stress, growth rate, etc) can be used to determine regimen of treatment of the plant, for example, additional introduction of the nucleic acid agent of the invention, augmentation of the treatment with other treatment modalities (e.g. insecticide, antibiotics, plant hormones, etc), or in order to determine timing of fruit harvest or irrigation times. Selection of plants for monitoring in a crop or field of plants can be random or systematic (for example, sentinel plants can be pre-selected prior to the treatment).

Polynucleotides delivered to plant cells by the methods and compositions of the invention, once within the plant tissues, can be taken up by other organisms associated with the plant, for example, by parasitic bacteria, fungi, protozoa or insects which utilize plant tissue for their benefit. For example, spread of RNAi products of dsRNA delivered to the plant via the methods of the invention can result in accumulation of biologically active siRNA and miRNA in plant tissues and fluids, such as pollen, leaves, stems, roots and other structures, fruit, flowers and the like. Organisms utilizing plants and plant structures, such as herbivorous insects and animals, plant parasites such as mites and nematodes, and even other plants, e.g. parasitic plants, can thus be exposed to the delivered polynucleotide(s), or their products. Thus, in some embodiments, the methods and compositions of the invention can be used to deliver an agrochemical molecule to a host organism, the method comprising contacting the plant cell with the agrochemical molecule and a cell wall degrading enzyme and at least one of a nucleic acid condensing agent, a transfection reagent, a surfactant, and a cuticle penetrating agent, thereby delivering the agrochemical molecule to the plant, and contacting the host organism with the plant, wherein said the organism ingests or imbibes cells, tissue or cell contents of the plant. As used herein, the term “agrochemical molecule” relates to any molecule having an effect on the metabolism, physiology, environment or functions of a plant. In some embodiments, the agrochemical molecule is a fertilizer, a pesticide, a fungicide, an antibiotic. In some embodiment, the agrochemical molecule is a dsRNA, a siRNA, a miRNA.

The compositions of the present invention can be provided in an agrochemical composition. Thus, according to some embodiments, there is provided an agrochemical composition comprising a composition of matter comprising a polynucleotide, a cell wall degrading enzyme and at least one of a nucleic acid condensing agent, a transfection agent, a surfactant and a cuticle penetrating agent. As used herein, the phrase “agrochemical composition” is defined as a composition for agrochemical use, and, as further defined, the agrochemical composition comprises at least one agrochemically active substance. Thus, in addition to the a polynucleotide, a cell wall degrading enzyme and at least one of a nucleic acid condensing agent, a transfection agent, a surfactant and a cuticle penetrating agent, the agrochemical composition of the present invention can include additional plant-beneficial or agrochemically active compounds. Exemplary plant-beneficial or agrochemically active compounds include, but not are limited to fertilizers, antibiotics, biocides, pesticides, pest repellents, herbicides, plant hormones, bacteriocides such as copper and the like. In some particular embodiments, the agrochemical composition comprises plant hormones. As used herein, the term “plant hormone” is used to indicate a plant-generated signaling molecule that normally affects at least one aspect of plant development, including but not limited to, growth, seed development, flowering and root growth. One of skill in the art will readily understand the term plant hormone and what entities fall under the scope of this term. For example, plant hormones include but are not limited to, abscisic acid (ABA) or a derivative thereof, gibberellins (GA), auxins (IAA), ethylene, cytokinins (CK), brassinosteroids (BR), jasmonates (JA), salicylic acid (SA), strigolactones (SL). In select embodiments, the fusion proteins of the present invention comprise a plant hormone binding domain that binds abscisic acid (ABA), gibberellins (GA), auxins (IAA) and/or jasmonates (JA).

Further, the agrochemical composition can optionally comprise one or more additives favoring optimal dispersion, atomization, deposition, leaf wetting, distribution, retardation of degradation by soil organisms and their secretion (for example, by addition of bacteriocides such as copper), retention and/or uptake of the agrochemical composition by the plant. As a non-limiting example such additives are diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents.

Exemplary concentrations of dsRNA in the composition include, but are not limited to, 0.01-0.3 μg/μl, 0.01-0.15 μg/μl, 0.04-0.15 μg/μl, 0.1-100 μg/μl; 0.1-50 μg/μl, 0.1-10 μg/μl, 0.1-5 μg/μl, 0.1-1 μg/μl, 0.1-0.5 μg/μl, 0.15-0.5 μg/μl, 0.1-0.3 μg/μl, 0.01-0.1 μg/μl, 0.01-0.05 μg/μl, 0.02-0.04 μg/μl, 0.001-0.02 μg/μl. According to further embodiments, the concentration of dsRNA in the treating solution includes, but is not limited to, 0.01-0.3 ng/μl, 0.01-0.15 ng/μl, 0.04-0.15 ng/μl, 0.1-100 ng/μl; 0.1-50 ng/μl, 0.1-10 ng/μl, 0.1-5 ng/μl, 0.1-1 ng/μl, 0.1-0.5 ng/μl, 0.15-0.5 ng/μl, 0.1-0.3 ng/μl, 0.01-0.1 ng/μl, 0.01-0.05 ng/μl, 0.02-0.04 ng/μl, 0.001-0.02 ng/μl. According to a specific embodiment, the concentration of the dsRNA in the treating solution is 0.1-1 μg/μl. According to some embodiments, the nucleic acid agent is provided in amounts effective to reduce or suppress expression of at least one plant pathogen resistance gene product. As used herein “a suppressive amount” or “an effective amount” refers to an amount of dsRNA which is sufficient to down regulate (reduce expression of) the target gene by at least 20%, 30%, 40%, 50%, or more, say 60%, 70%, 80%, 90% or more even 100%.

According to some embodiments of the present invention, the concentration of dsRNA is provided to the plant in effective amounts, measured in mass/kg plant. Such effective amounts include, but are not limited to, 0.001-0.003 mg/kg, 0.005-0.015 mg/kg, 0.01-0.15 mg/kg, 0.1-100 mg/kg; 0.1-50 mg/kg, 0.1-10 mg/kg, 0.1-5 mg/kg, 0.1-1 mg/kg, 0.1-0.5 mg/kg, 0.15-0.5 mg/kg, 0.1-0.3 mg/kg, 0.01-0.1 mg/kg, 0.01-0.05 mg/kg, 0.02-0.04 mg/kg, 0.001-0.02 mg/kg, 0.001-0.003 g/kg, 0.005-0.015 g/kg, 0.01-0.15 g/kg, 0.1-100 g/kg; 0.1-50 g/kg, 0.1-10 g/kg, 0.1-5 g/kg, 0.1-1 g/kg, 0.1-0.5 g/kg, 0.15-0.5 g/kg, 0.1-0.3 g/kg, 0.01-0.1 g/kg, 0.01-0.05 g/kg, 0.02-0.04 g/kg, 0.001-0.02 g/kg plant. According to a specific embodiment, the effective amount of the dsRNA provided to the plant is 0.0001-10000 mg/kg plant. In another embodiment, the effective amount is 1-1000 mg/kg plant.

The compositions and agrochemical compositions of the present invention are suitable for agrochemical use. “Agrochemical use,” as used herein, not only includes the use of agrochemical compositions as defined above that are suitable and/or intended for use in field grown crops (e.g., agriculture), but also includes the use of agrochemical compositions that are meant for use in greenhouse grown crops (e.g., horticulture/floriculture) or hydroponic culture systems or uses in public or private green spaces (e.g., private gardens, parks, sports fields), for protecting plants or parts of plants, including but not limited to bulbs, tubers, fruits and seeds (e.g., from harmful organisms, diseases or pests), for controlling, preferably promoting or increasing, the growth of plants; and/or for promoting the yield of plants, or the parts of plants that are harvested (e.g., its fruits, flowers, seeds etc.).

“Agrochemical active substance,” as used herein, means any active substance or principle that may be used for agrochemical use, as defined above. Examples of such agrochemical active substances will be clear to the skilled person and may for example include compounds that are active as insecticides (e.g., contact insecticides or systemic insecticides, including insecticides for household use), acaricides, miticides, herbicides (e.g., contact herbicides or systemic herbicides, including herbicides for household use), fungicides (e.g., contact fungicides or systemic fungicides, including fungicides for household use), nematicides (e.g., contact nematicides or systemic nematicides, including nematicides for household use) and other pesticides (for example avicides, molluscicides, piscicides) or biocides (for example, agents for killing bacteria, algae or snails); as well as fertilizers; growth regulators such as plant hormones; micro-nutrients, safeners; pheromones; repellants; baits (e.g., insect baits or snail baits); and/or active principles that are used to modulate (i.e., increase, decrease, inhibit, enhance and/or trigger) gene expression (and/or other biological or biochemical processes) in or by the targeted plant (e.g., the plant to be protected or the plant to be controlled). Agrochemical active substances include chemicals, but also nucleic acids, peptides, polypeptides, proteins (including antigen-binding proteins) and micro-organisms. Examples of such agrochemical active substances will be clear to the skilled person; and for example include, without limitation: Diamides: chlorantraniliprole, cyantraniliprole, flubendiamide, tetronic and tetramic acid derivatives: spirodiclofen, spirotetramat, spiromisifen, modulators of chordotonal organs: pymetrozine, flonicamid; nicotinic acetylcholine receptor agonists: sulfoxaflor, flupyradifurone; spiroxamines, glyphosate, paraquat, metolachlor, acetochlor, mesotrione, 2,4-D,atrazine, glufosinate, sulfosate, fenoxaprop, pendimethalin, picloram, trifluralin, bromoxynil, clodinafop, fluoroxypyr, nicosulfuron, bensulfuron, imazetapyr, dicamba, imidacloprid, thiamethoxam, fipronil, chlorpyrifos, deltamethrin, lambda-cyhalotrin, endosulfan, methamidophos, carbofuran, clothianidin, cypermethrin, abamectin, diflufenican, spinosad, indoxacarb, bifenthrin, tefluthrin, azoxystrobin, thiamethoxam, tebuconazole, mancozeb, cyazofamid, fluazinam, pyraclostrobin, epoxiconazole, chlorothalonil, copper fungicides, trifloxystrobin, prothioconazole, difenoconazole, carbendazim, propiconazole, thiophanate, sulphur, boscalid and other known agrochemicals or any suitable combination(s) thereof. Other suitable agrochemicals will be clear to the skilled person based on the disclosure herein, and may for example be any commercially available agrochemical, and for example include each of the compounds listed in any of the websites of the Herbicide Resistance Action Committee, Fungicide Resistance Action Committee and Insecticide Resistance Action Committee, as well as those listed in Phillips McDougall, AgriService November 2007 V4.0, Products Section—2006 Market, Product Index pp. 10-20. The agrochemical active substances can occur in different forms, including but not limited to, as crystals, as micro-crystals, as nano-crystals, as co-crystals, as a dust, as granules, as a powder, as tablets, as a gel, as a soluble concentrate, as an emulsion, as an emulsifiable concentrate, as a suspension, as a suspension concentrate, as a suspoemulsion, as a dispersion, as a dispersion concentrate, as a microcapsule suspension or as any other form or type of agrochemical formulation clear to those skilled in the art. Agrochemical active substances not only include active substances or principles that are ready to use, but also precursors in an inactive form, which may be activated by outside factors. As a non limiting example, the precursor can be activated by pH changes, caused by plant wounds upon insect damage, by enzymatic action caused by fungal attack, or by temperature changes or changes in humidity.

The agrochemical composition hereof may be in a liquid, semi-solid or solid form and for example be maintained as an aerosol, flowable powder, wettable powder, wettable granule, emulsifiable concentrate, suspension concentrate, microemulsion, capsule suspension, dry microcapsule, tablet or gel or be suspended, dispersed, emulsified or otherwise brought in a suitable liquid medium (such as water or another suitable aqueous, organic or oily medium) for storage or application. Optionally, the composition further comprises one or more further components such as, but not limited to diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents or the like, suitable for use in the composition hereof.

According to some aspects of the present invention, there is also provided a method for manufacturing an agrochemical composition, the method comprising (i) selecting at least one, preferably more, polynucleotides, a cell wall degrading enzymes and at least one of a nucleic acid condensing agent, a transfection agent, a surfactant and a cuticle penetrating agent, and (ii) formulating the polynucleotide, cell wall degrading enzyme and at least one of a nucleic acid condensing agent, a transfection agent, a surfactant and a cuticle penetrating agent in a compound with additional substance or substances, such as an agrochemical active substance, or a combination of compounds, and optionally (iii) adding further components that may be suitable for such compositions, preferably for agrochemical compositions. In some embodiments, the compound is comprised in a carrier.

Reagents of the present invention can be packed in a kit including the composition of the invention, instructions for introducing the composition of the invention into the plants and optionally an agrochemically active agent.

Compositions of some embodiments of the invention may, if desired, be presented in a pack or dispenser device, which may contain one or more dosage forms containing the active ingredient. The pack may, for example, comprise metal or plastic foil, such as a blister pack. The pack or dispenser device may be accompanied by instructions for introduction to the plant.

According to an exemplary embodiment, the polynucleotide, or composition and additives are comprised in separate containers.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Plant Preparation and Growth Conditions

Tomato Tiny Tim seeds were germinated in water-saturated germination soil mixture in germination cones, cones covered to exclude light and incubated for 48-72 hours at 23-26 degrees C., then transferred to 16/8 hour light/dark cycle. Seedlings appeared typically after 5 days. The seedlings were then grown to the four true leaf stage (approximately 3 weeks post germination). Citrus plants were grown using 12 month old rootstocks and 6 months old scions and grown at a green house.

dsRNA Synthesis

dsRNA preparation was performed by standard methods, for example, using the Ambion® MEGAscript® RNAi Kit. dsRNA integrity is verified on gel and purified by a column based method. The concentration of the dsRNA is evaluated both by Nano-drop and gel-based estimation. dsRNA is dissolved in nuclease free water to a final concentration of 10 mg/ml. The purified dsRNA, further to a final concentration of 100-1000 ng/μl, serves for the following experiments.

Peptide Synthesis

(KH)9-Bp100 (KHKHKHKHKHKHKHKHKHKKLFKKILKYL-NH 2) (SEQ ID NO: 21), theoretical pI/Mw: 10.81/3809.71 Da) and IR9 (GLFEAIEGFIENGWEGMIDGWYGRRRRRRRRR)(SEQ ID NO: 22) theoretical pI/Mw: 11.86/3996.55 Da was synthesized using standard 9-fluorenylmethoxycarbonyl (Fmoc) solid-phase peptide synthesis (Fields and Noble, 1990). The polypeptides were purified using high-performance liquid chromatography (HPLC), and the molecular weights were confirmed by matrix-assisted laser desorption/ionization-time-of-flight (MALDI-TOF) mass spectrometry. Peptides were dissolved in nuclease free water to a final concentration of 200-1000 mM as mentioned.

Characterization of Peptide-dsRNA Complexes

Peptide-dsRNA complexes were prepared so that the N/P ratio (ratio of amine groups in the peptide to phosphate groups in the nucleic acid) between the peptide and dsRNA ranges from 0.1-10. The peptide positive charge is calculated by the number of amino acids which are positively charged at neutral pH. The dsRNA negative charges are calculated assuming that each nucleotide carriers 1 negative charge.

To prepare peptide-dsRNA complexes, 1 mM peptide solution is added to dsRNA solution while vortexing in ddH2O or sodium phosphate buffer pH 6.8, as mentioned. Complexes are then incubated at RT for 15 min.

Formation of complexes is verified using a retardation assay. 500 ng of dsRNA were separated on 1% agarose gel for 45 min at 80V. When dsRNA is completely bound by the peptide in a high molecular weight complex, with charges neutralized, it is retarded (will not migrate on the gel).

Preparation of Cell Wall Degrading Enzymes (CWDE)

When cell wall degrading enzymes are used (SIGMA, cat. # D9515), they are dissolved in nuclease free water to a final concentration of 1 mg/ml and let stand at room temperature for 30 minutes, to let insolubilized material sediment.

To test the stability of the peptide-dsRNA complexes in the presence of the CWDE, CWDE are added to the complexes and then, 500 ng of the dsRNA in complex formulation are separated on 1% agarose gel as describe above.

Protoplast Assay (CWDE Activity)

CWDE were diluted 10 fold in 0.625M sucrose solution. Tiny Tim tomato plant leaves were cut into equally sized pieces and placed in 12 well plate. In each well, 1 ml of cell wall degrading enzyme solution was added. The plate was shaken gently at RT over night. Later, formation of protoplasts was detected using a microscope.

Treatment of Tomato Plants with Peptide-dsRNA Complexes

18 d post-seeding Tiny Tim plants were treated with a peptide-dsRNA complex either by irrigation or topical application following spraying.

For administration of the complex by irrigation, plants are removed from pots and as much medium removed as possible. Roots are washed twice with tap water and cut diagonally, to cut both the main and lateral roots. Plants are then dried for 30 min at room temperature (in 25° C.) and placed in an Eppendorf tube containing 1 ml of the indicated solution, under red light and a 16:8 hour D:L cycle until all the solution had been taken up.

When treated by spraying and topical administration, 3 week old Tiny Tim tomato plants are transplanted to bigger pots 24 hr prior to treatment. Treated leaves are either sprayed with carborundum suspension (50 mg in 100 ml of ddH2O) or mineral oil (such as 1% Eco oil spray (EOS) (ADAMA SK EnSpray 99)) at 10-40 PSI using an air brush sprayer. Immediately after spraying, about 50 ul from the formulation is applied on the selected leaf(s). Plants are kept under red light and 16:8 hour D:L cycle. Treated leaves were cut at selected time points and immediately frozen in liquid nitrogen for further RNA extraction.

Treatment of Citrus Trees with Peptide dsRNA-Complexes

Citrus plants are treated with peptide-dsRNA complex (2000 molar ratio) either by injection or topical application following spraying.

For application by injection, the following protocol is used:
1. Prepare a well irrigated tree for application between 10-11 μm.
2. Place the tree in a sunny area but not exposed to extreme heat conditions.
3. Point of entry is 40-80 cm above ground level.
4. For a 10-15 mm trunk use an auger type 3 mm diam. drill type and drill a 10 mm deep hole at a low speed.
5. Change to a 4 mm drill and widen the hole.
6. Attach the plastic adaptor to the tree trunk.
7. Cut a 20 cm long piece of latex tube, 4 mm internal diam.
8. Fill a 30 ml syringe with the chosen solution.
9. Attach the latex tube to the syringe and fill the tube with solution.
10. Attach the tube to the adaptor and empty the solution in the tube.
11. Add additional solution volume to reach a pressure of 60-80 kPa.
12. After 9 hours from application, solution may be found in leaves and after 24 hours in the roots.

When spraying, plants are sprayed with mineral oil (such as 1% Eco oil spray (EOS) (ADAMA SK EnSpray 99) until run off. 1.5 hr later, the trees are washed in excess water and dried. Then, a complex of peptide-dsRNA formulated with the CWDE is topically applied to the treated leaves. At predetermined intervals, treated leaves were cut and immediately frozen in liquid nitrogen for further RNA extraction.

Sample Spraying Procedure

1. Hold the leaf with your free hand with the petiole between your index and middle finger, and the middle and ring finger supporting the blade. This is the best position to keep the leaf stable when spraying.

2. Spraying should be done as close as possible perpendicular (at 90 degrees with respect to the blade) to the leaf.

3. Spray at a distance of 4-5 cm (2 inch) from the blade at 5-10 PSI when spraying Oil (EOS) or 40 PSI when spraying carborundum depending on the type on nozzle used—full cone, hollow cone or fan type nozzles or hand held spray guns (the manufacturer's specifications regarding distance from leaf & pressure should be adhered to for each type of nozzle).

4. The duration of the spray should be about 1-1.5 seconds per leaf.

Quantitative RT-PCR to Measure Gene Knockdown

In order to monitor the levels of target mRNA in the treated plants, quantitative PCR analysis was performed on the RNA extracted from homogenized plant material samples, following synthesis of cDNA copies using reverse transcriptase.

The cDNA from each replicate treatment was then used to assess the extent of RNAi by measuring levels of gene expression using qRT-PCR. Reactions were performed in triplicate and compared to an internal reference to determine relative abundance of transcripts (expression levels).

TABLE 3
List of primers

| | | | |
|---|---|---|---|
| Le_Actin_qRT_F | SEQ ID | qPCR of tomato Actin (HKG) | TTGCTGACCGTATGAGCAAG |
| | NO: 11 | | |
| Le_Actin_qRT_R | SEQ ID | | GGACAATGGATGGACCAGAC |
| | NO: 12 | | |
| EF1_2F | SEQ ID | qPCR for EF (citrus HKG) | GTTCTTCACGTTGAAGCCAA |
| | NO: 13 | | |
| EF1_2R | SEQ ID | | GTCCTCAAGCCTGGTATGGT |
| | NO: 14 | | |
| cGPT_F | SEQ ID | qPCR for citrus GPT | TTCGTGTGGTGGGTAGC |
| | NO: 15 | | |
| cGPT_R | SEQ ID | | GAGCATTGACGGGTTGA |
| | NO: 16 | | |
| Le_PDS_F | SEQ ID | qPCR for tomato PDS | GGCTACGTCCAACAAGTAACTCA |
| | NO: 17 | | |
| Le_PDS_R | SEQ ID | | CCGGTGACTACACGAAACAG |
| | NO: 18 | | |
| Le_Agpase_F | SEQ ID | qPCR for tomato agpase | AACCATATTGACAGAAATGCTGATA |
| | NO: 19 | | |
| Le_Agpase_R | SEQ ID | | CAGCCCAAAATCTGATGCTC |
| | NO: 20 | | |

### Results

### Example I: Characterization of Peptide:dsRNA Complexes

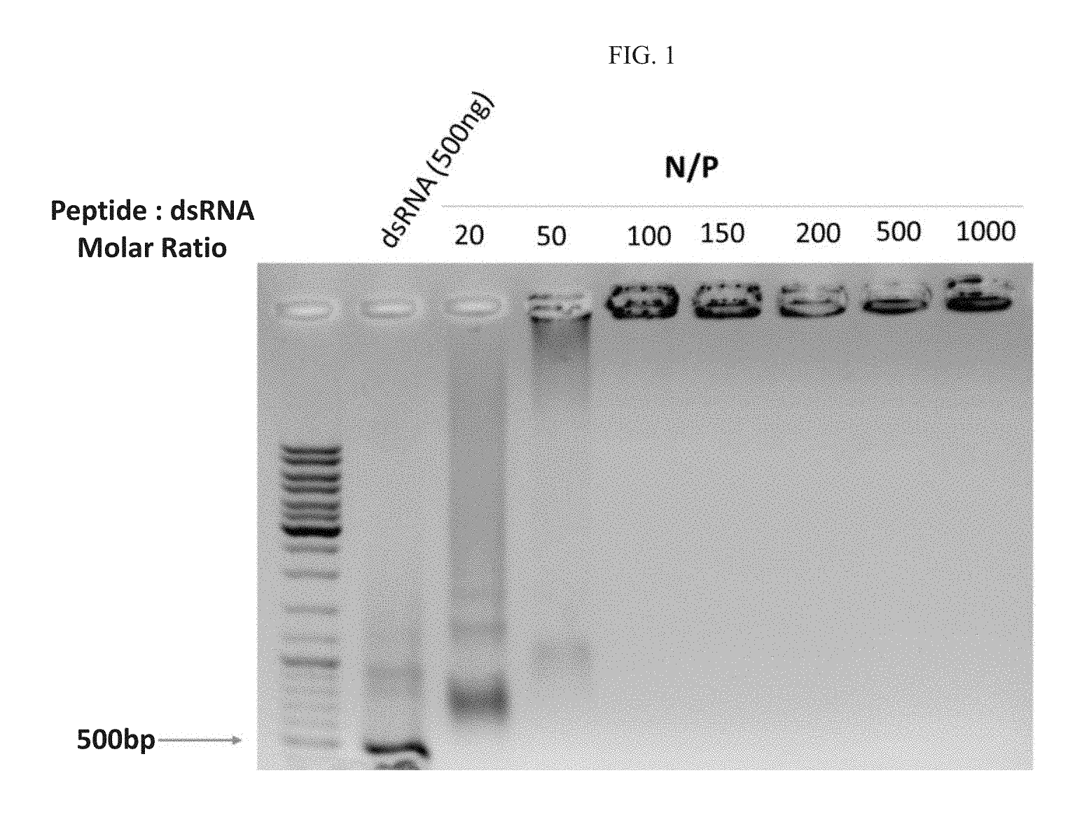

In order to verify the complexation of the positively charged peptide with the negatively charged dsRNA, complexes with different molar ratios were separated on a gel. When the negative charges are completely masked by the positive charges, high molecular weight complexes are expected not to migrate on the gel and the dsRNA will be retained in the well. For example, in FIG. 1, it can be seen that a molar ratio of 100 and higher between the peptide and the dsRNA in ddH2O results in full masking of the dsRNA negative charge and no dsRNA migration into the gel is observed.

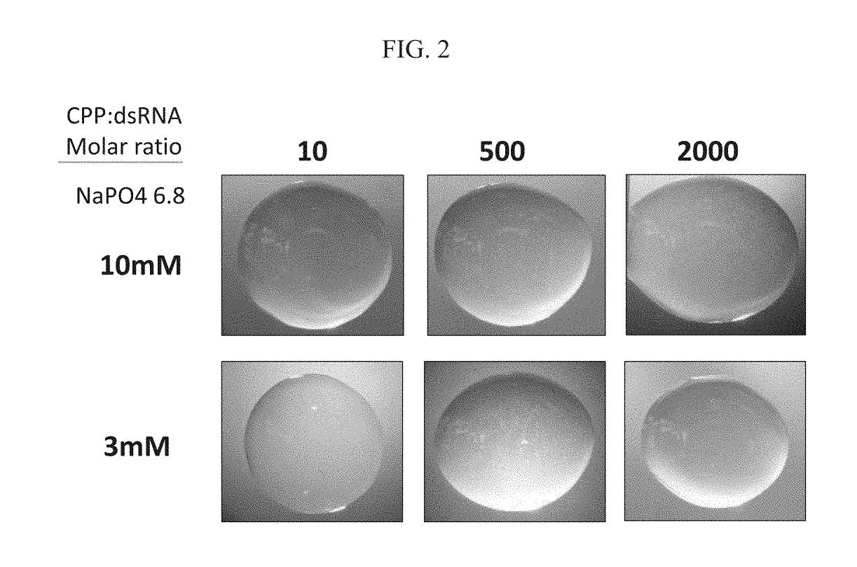

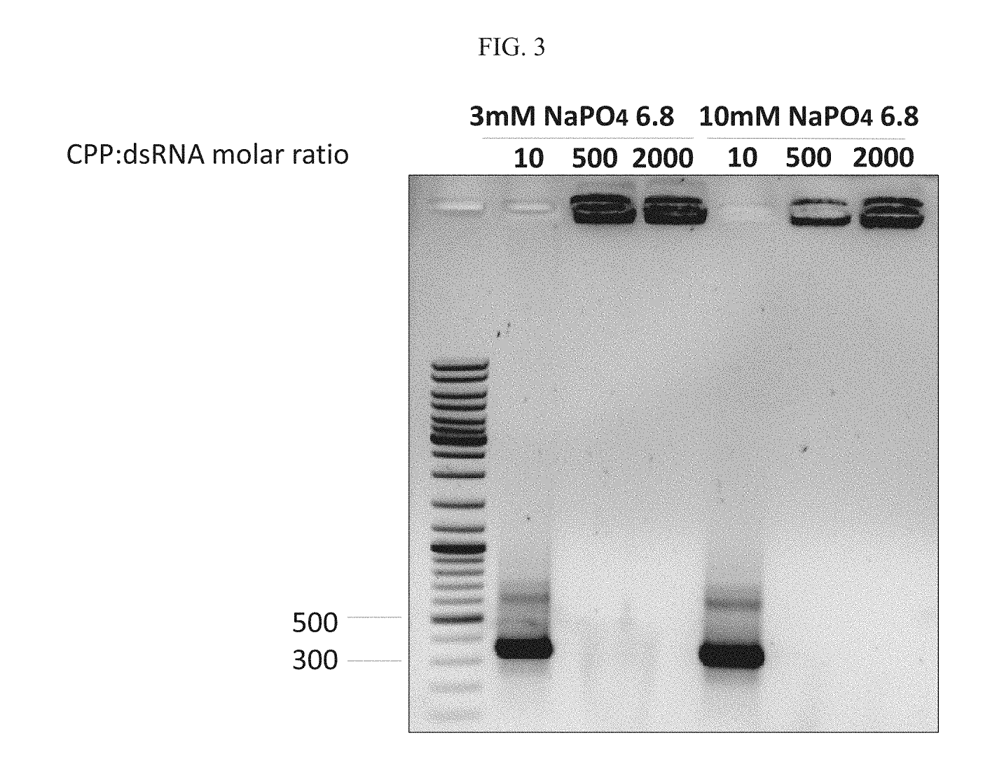

Formation of peptide:dsRNA complex was also tested in different concentrations of the sodium phosphate buffer. Formation of aggregates was evaluated using a binocular, and was seen in 10 mM sodium phosphate buffer in molar ratios of 500 and 2000. However, in 3 mM sodium phosphate buffer, at 2000 molar ratio, a clear solution was detected (FIG. 2). To verify complex formation in all these groups, they were separated on a gel (FIG. 3), and complexation was detected as a band that did not migrate on the gel, suggesting that in 3 mM phosphate buffer even though aggregates are not seen, complexes were indeed formed.

### Example II: Cell Wall Degrading Enzyme (CWDE) Toxicity

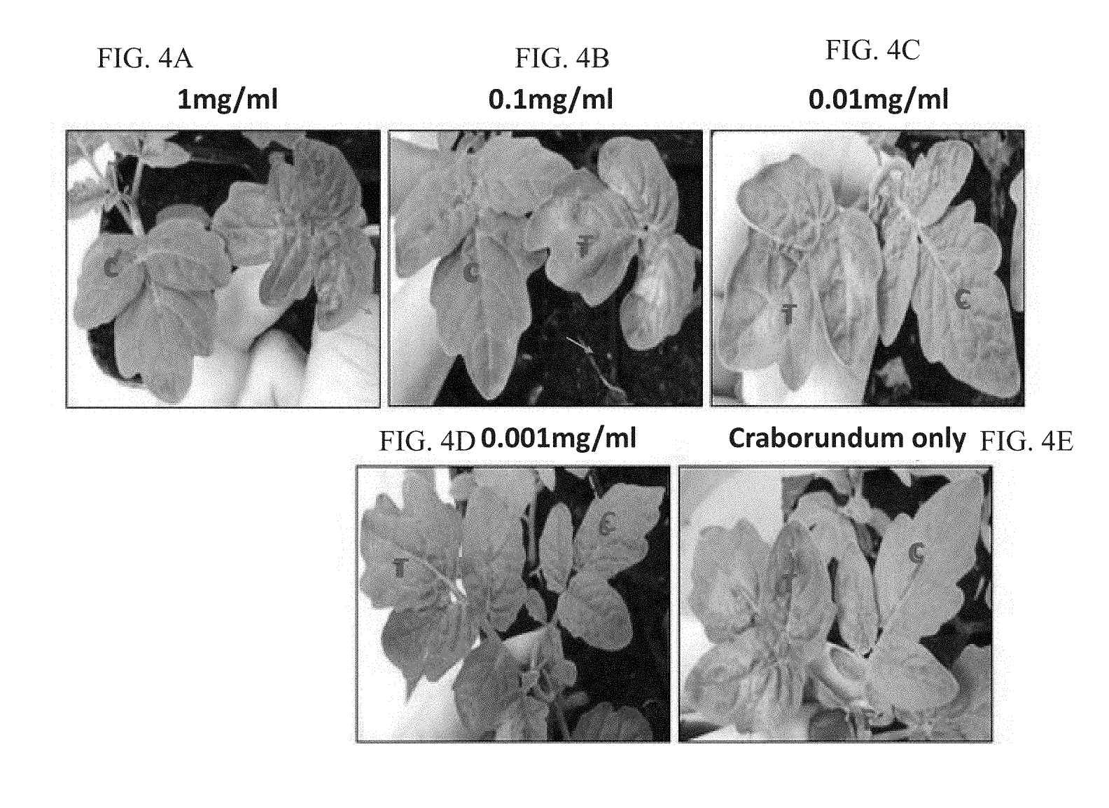

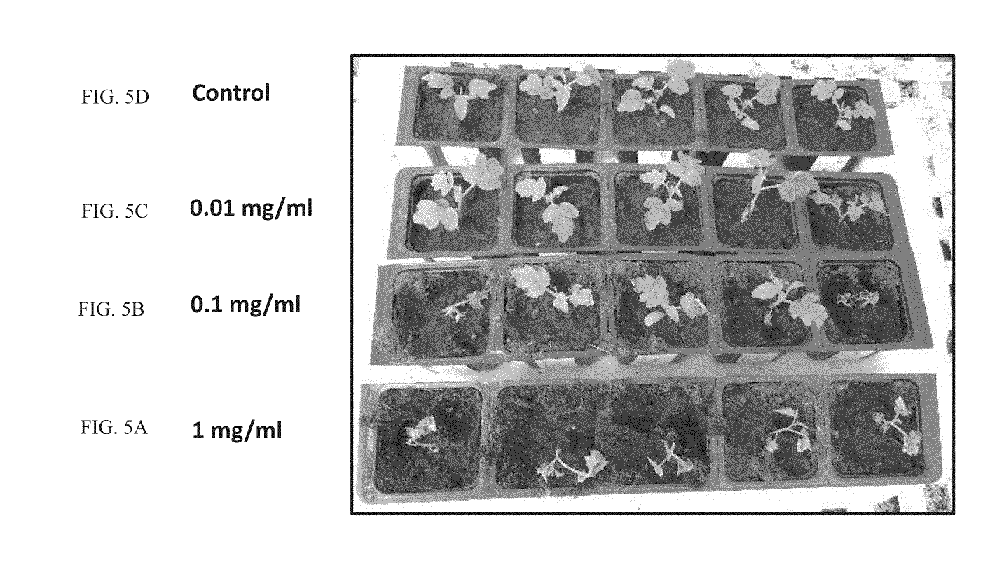

To select the CWDE concentration and solvent in which no severe toxic effects can be detected on tomato plants through topical application following topical application following spraying (FIGS. 4A-4E) or irrigation (FIGS. 5A-5D), increasing concentrations of CWDE in ddH2O were applied to plants which were monitored for toxic effects for 5 days. It can be seen, that at concentrations of 1-0.1 mg/ml, toxicity of the CWDE can be detected as scorching of the leaves in treated (T) leaves compared to the untreated control (C) (FIGS. 4A and 4B). When applied via irrigation, these high CWDE concentrations caused plant mortality (FIGS. 5A and 5B).

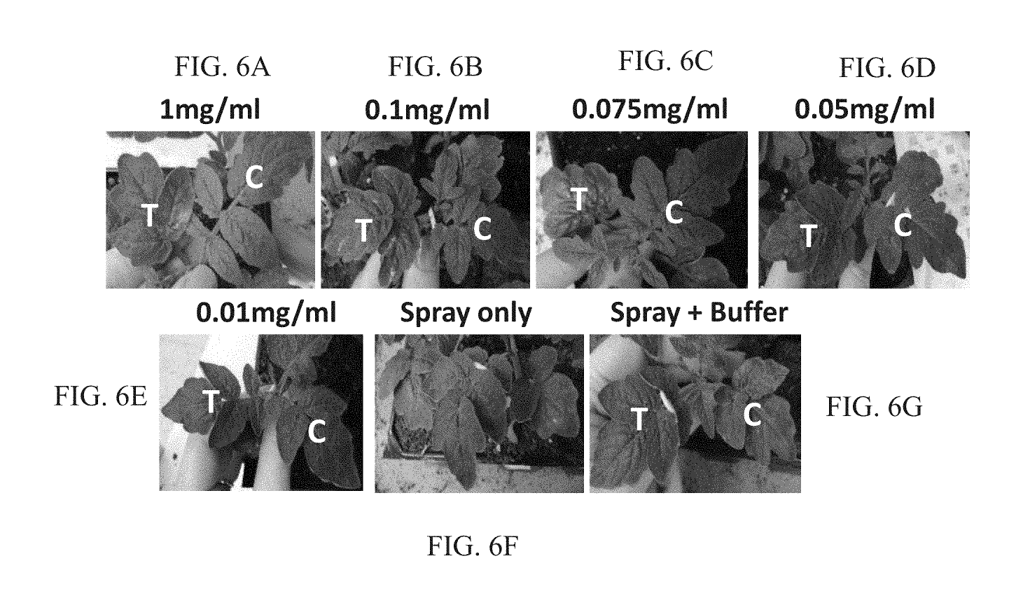

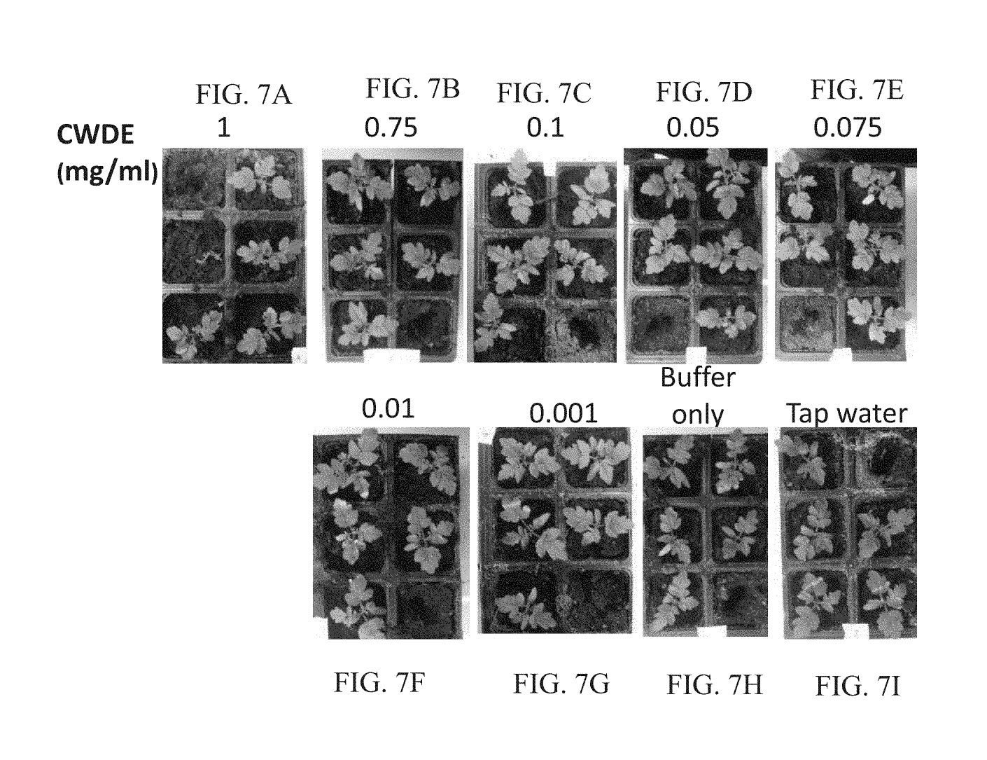

The effect of CWDE dissolved in a sodium phosphate buffer was assessed in the same manner. When the CDWE in sodium phosphate buffer was applied by topical administration following spraying (FIGS. 6A-6G), no severe toxic effects were detected in any of the treatment groups. When applied by irrigation (FIGS. 7A-7I), toxic effects of the CWDE were detected only at a concentration of 1 mg/ml (FIG. 7A).

### Example III: Stability of Complexes with CWDE

To test whether the peptide:dsRNA complexes were stable in the presence of the CWDE, complex stability was tested in various solvents (ddH2O, PBS, Sodium phosphate buffer) on a gel. Stable complexes are expected to appear as a band in the well, while when disassembly of the complexes occurs, migration of the dsRNA on the gel will be observed.

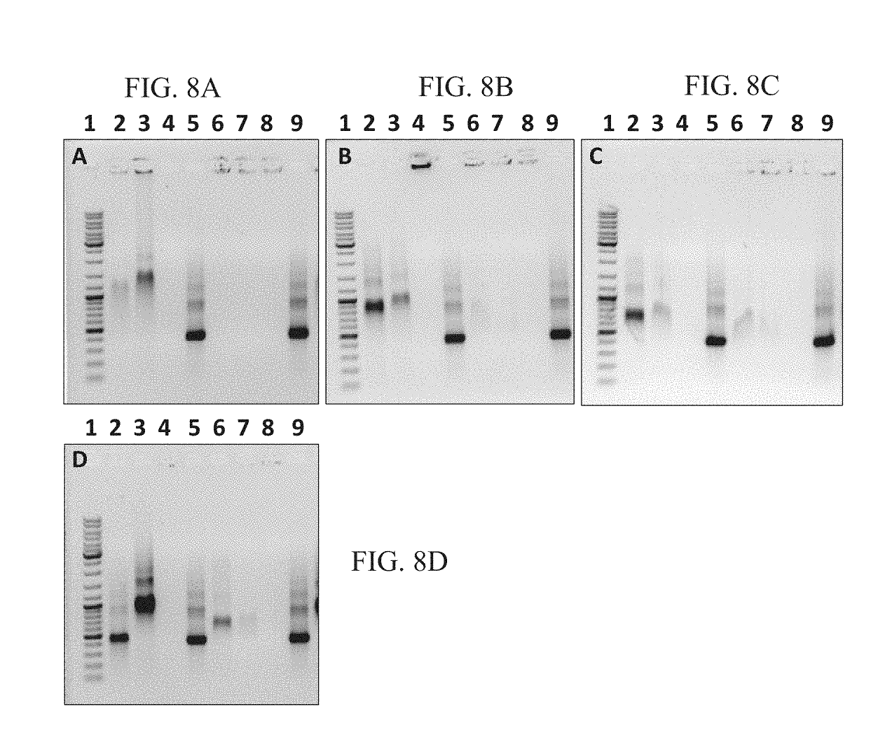

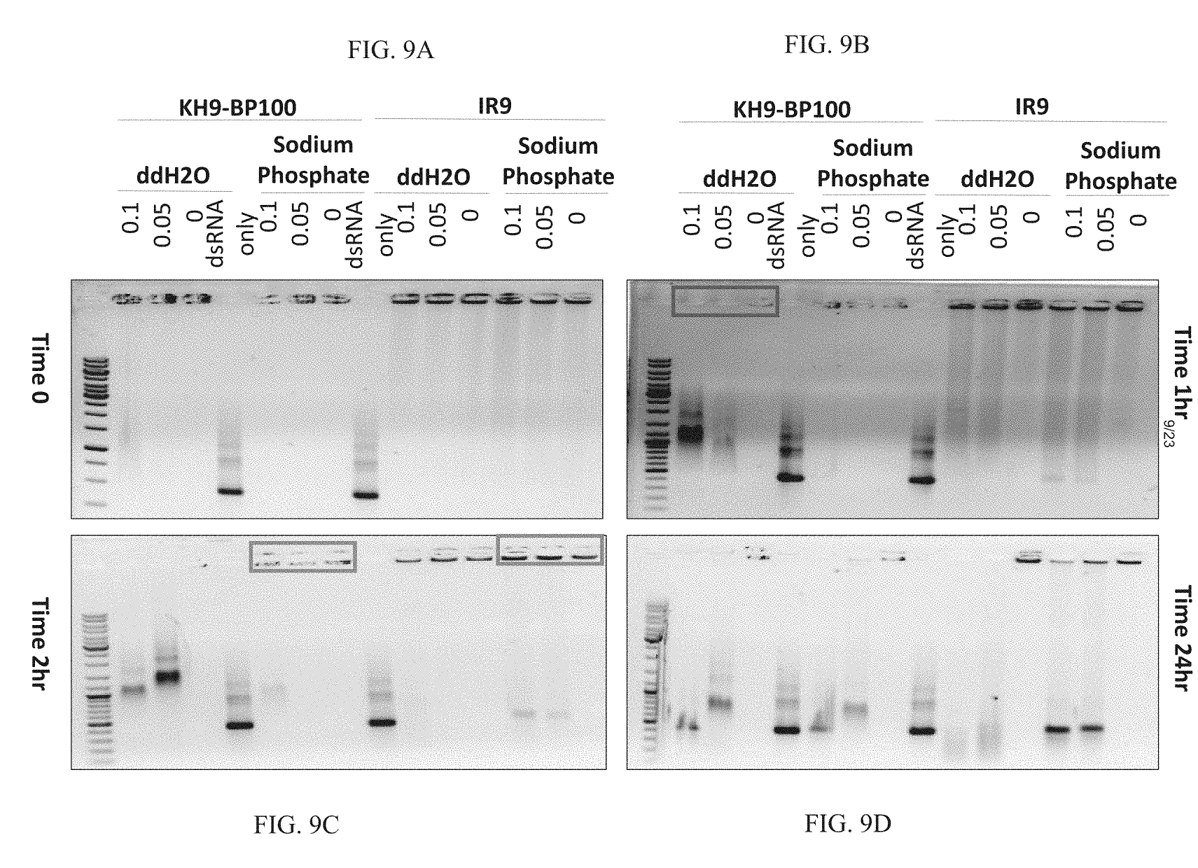

Surprisingly, the lowest stability was detected in ddH2O, as complexes were degraded as soon as CWDE was added (time 0, FIG. 8A, lanes 2-4 and 6-8), as can be seen in migration of the dsRNA bands from the wells. In PBS (FIG. 8C, lanes 2-4 and 6-8) or sodium phosphate (FIG. 9C, sodium phosphate), stability of the complexes was detected up to 2 hr after the addition of the CWDE.

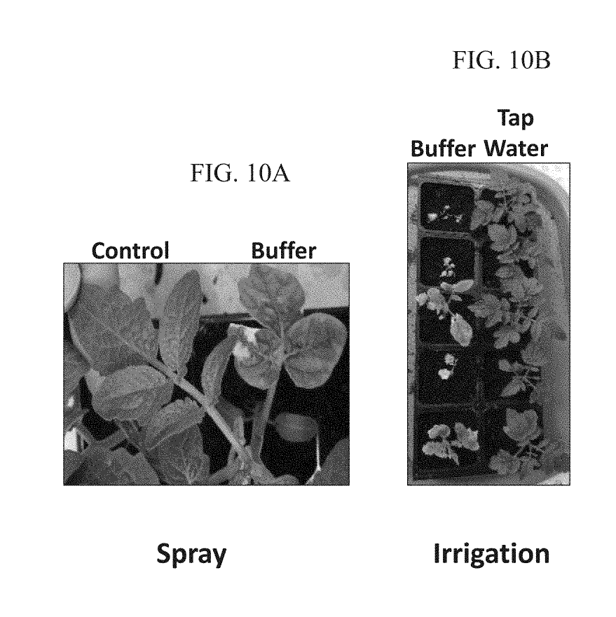

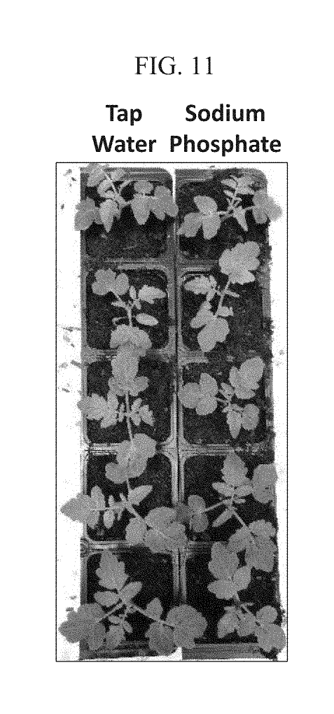

However, PBS proved to be toxic to tomato plants when applied by topical application following carborundum spraying or by irrigation (FIGS. 10A-10B), while no toxic effects were observed when sodium phosphate served as buffer for irrigation application (FIG. 11) or by topical application following carborundum spraying (results not shown).

### Example IV: CWDE Activity in Sodium Phosphate Buffer

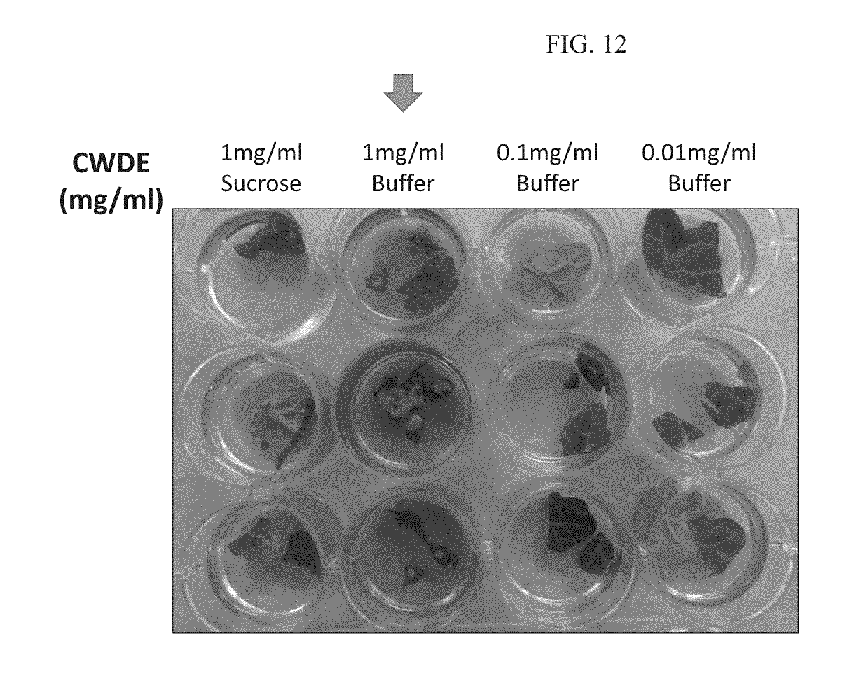

The activity of the CWDE in sodium phosphate buffer was verified through a protoplast assay (FIG. 12). Active CWDEs degrade the cell wall and release protoplasts (cells without a cell wall) into the media. These protoplasts can be detected using a microscope and by the change of color of the media. In sodium phosphate, CWDE activity was detected down to 0.1 mg/ml (FIG. 12).

Further, the activity of the CWDE was examined in the presence of the KH9-BP100 peptide (SEQ ID NO: 21):dsRNA complexes in three molar ratios (20, 200, 2000) (results not shown) and the enzymes were still found active at 1 and 0.1 mg/ml CWDE.

A secondary toxicity assay, using both CWDE at the selected concentration of 0.1 mg/ml and dsRNA:peptide complexes in different molar ratios, was performed to evaluate any toxic effects of the combination (complexes and CWDE) on the tomato plants through topical application following spraying and no severe toxic effects were detected in any of the treatment groups (results not shown).

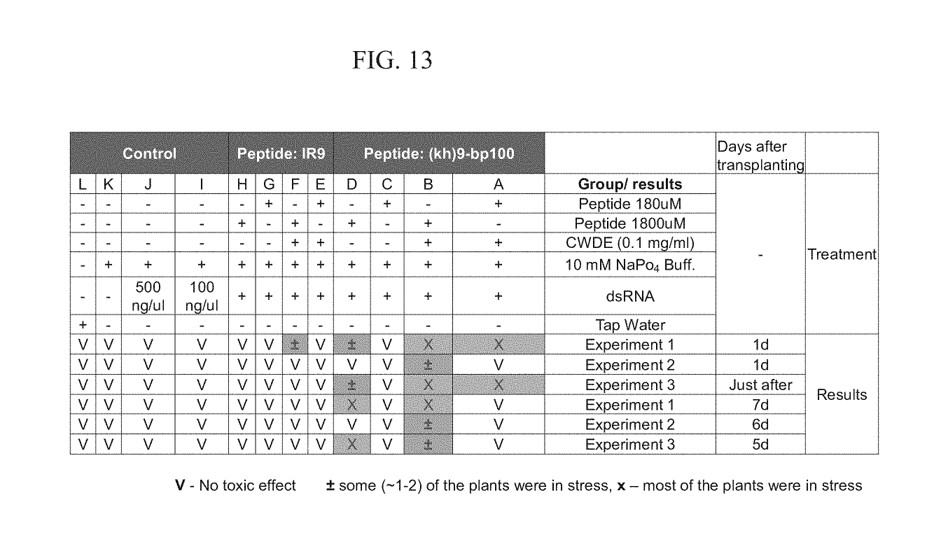

This secondary toxicity assay was also performed through irrigation (FIG. 13) where some toxic effects were detected only when using the KH9-BP100 peptide (SEQ ID NO: 21).

### Example V: Gene Down Regulation in Response to Topical Application of dsRNA-Peptide-CWDE Following Spraying

After determining a suitable CWDE concentration (0.1 mg/ml) and peptide:dsRNA molar ratio (200/2000) in the selected buffer (sodium phosphate) (i.e. where stability of complexes has been demonstrated and no severe toxic effects were detected), the effect of application of topical application of the full formulation (following spraying) on tomatoes and on citrus plants was evaluated using qPCR.

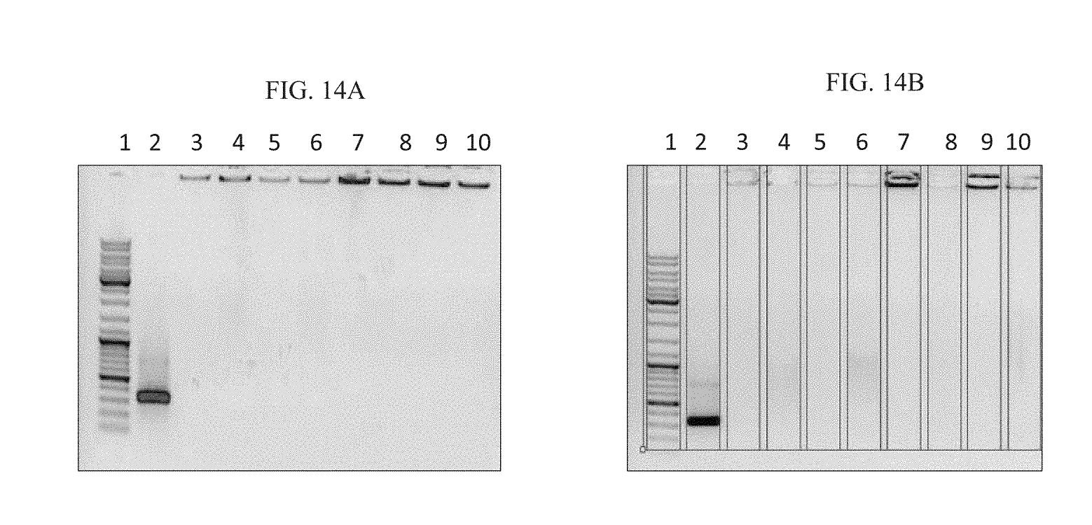

Two different methods to penetrate the cuticle were tested: either an abrasive element in the form of carborundum spraying or the use of a narrow range mineral oil (e.g. SK EnSpray 99). Stability of the complexes in the presence of the oil was verified on a gel (FIGS. 14A-14B lanes 5, 6 and 9, 10), and to ensure that the CWDE are not inactivated by the presence of the oil, the oil and complexes were applied separately onto the leaves with the oil being applied first.

In FIG. 15A, it can be seen that 24 hr post treatment about 40% reduction in PDS mRNA levels were detected in response to application of the PDS-specific dsRNA-peptide-CWDE formulation after carborundum spray. This reduction in PDS mRNA levels lasted for at least 24 hr more (FIG. 15B).

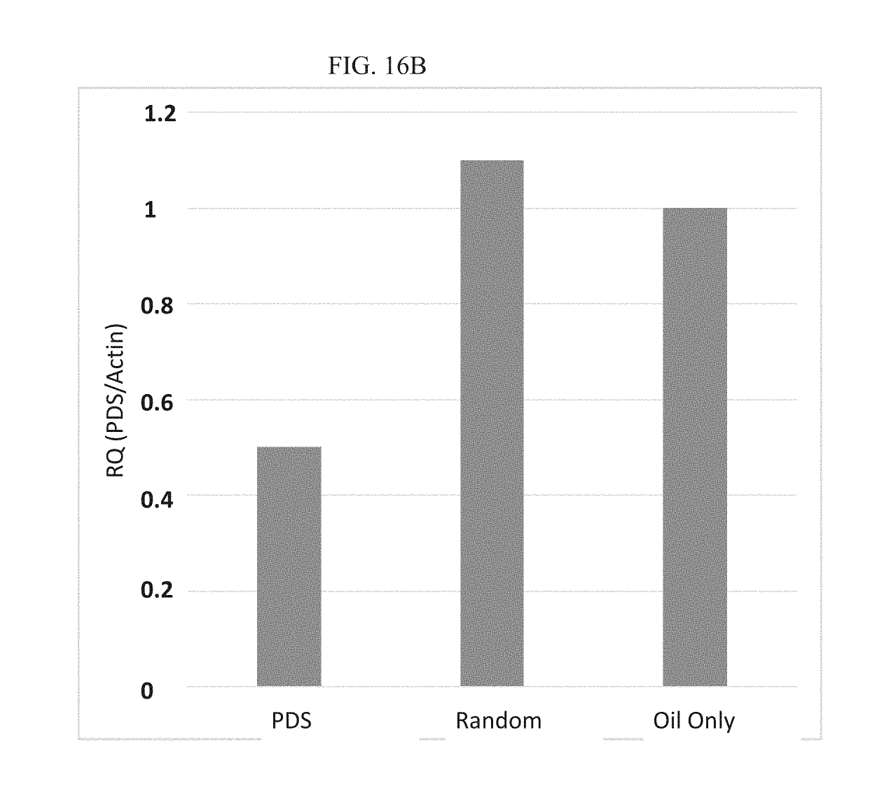

Reduction in AGPase and PDS mRNA levels were also detected in response to application of AGPase- and PDS-specific dsRNA-peptide-CWDE formulation after spray with mineral oil (e.g. SK EnSpray 99 oil) (FIGS. 16A-16B).

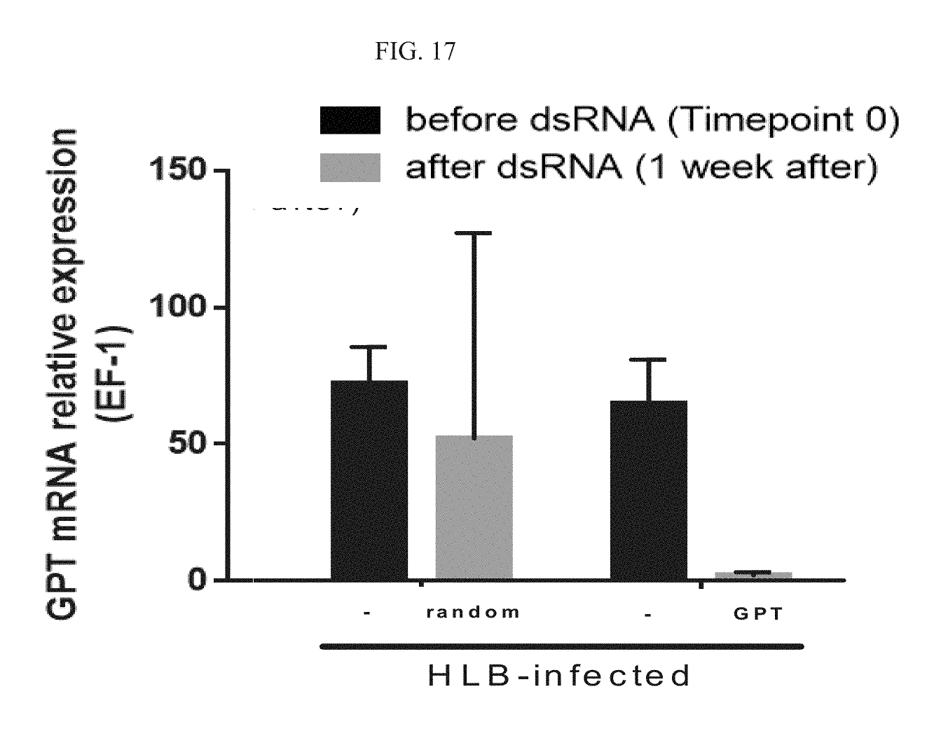

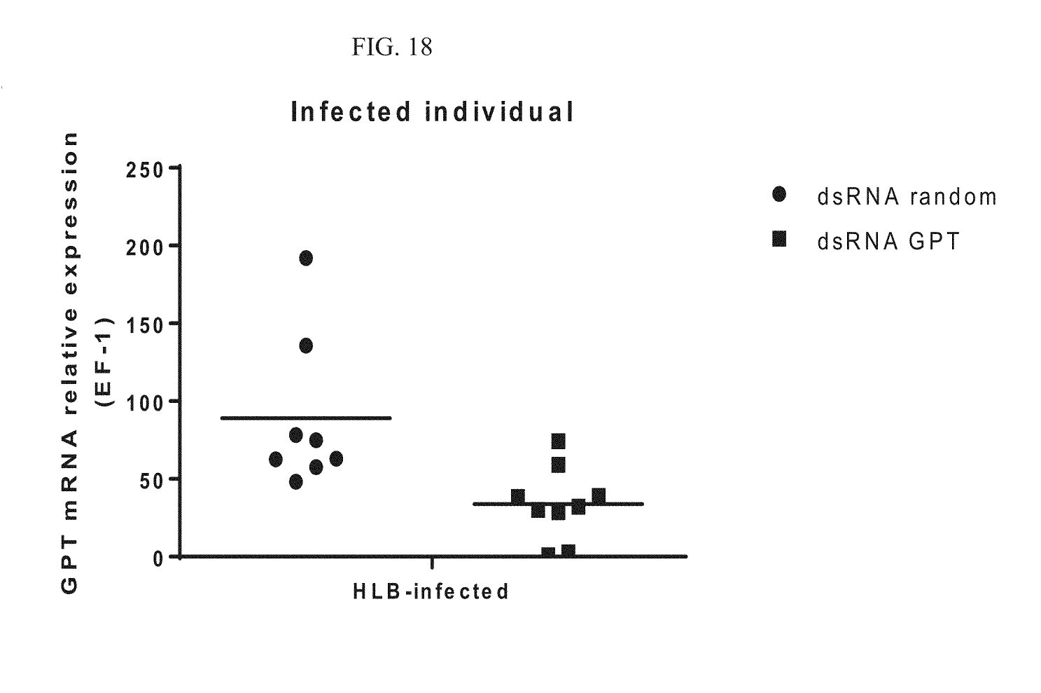

To further test the efficacy of the complexes in gene down regulation, GPT-specific dsRNA-peptide-CWDE complexes (FIG. 17) or 200 fold more naked GPT-specific dsRNA (FIG. 18) were administered by injection into the tree. Despite the huge difference in amount of dsRNA delivered (0.5% delivered as a complex, compared to the naked dsRNA), a greater degree of gene down regulation (orders of magnitude greater) was detected with injection of the complexes (about 50 times less relative expression), compared to the downregulation achieved naked dsRNA injection (approx two times less relative expression) in a disease model (HLB) which causes upregulation of GPT compared to uninfected trees.

Taken together, these results indicate that complexing dsRNA with polycationic peptides such as the peptides KH9-Bp100 (SEQ ID NO: 21) and IR9 (SEQ ID NO: 22) used here, and cell wall degrading enzymes, in a suitable buffer, applied topically in conjunction with additional preparation (e.g. carborundum and oil spraying, in the presence of surfactant) or when provided by injection, can significantly enhance the efficacy of delivery of bioactive polynucleotides to the cells of plants.

### Example VI: Downregulation of CalS Expression for Treating Candidatus (Ca.) Liberibacter solanacearum” (LSO) Infection in Tomato

Materials and Methods

Infection of Tomato Plants with LSO

Tomato plants inoculated at 25° C.±1 were gently wrapped at the base of the petiole of the lowest leaf with a small amount of cotton fiber (taken from a cotton ball) in order to create a flexible seal. The opening of a nylon mesh organza bag was placed over the leaf and closed over the cotton by pulling on the drawstrings.
15 adult psyllids were aspirated. The other end of the aspirator was inserted into the opening of the bag, pulling the drawstrings to cinch up snugly. The psyllids were gently blown into the bag. The aspirator tube was removed and further cinched by pulling the drawstrings snugly in order to prevent escape of psyllids.
Test and matching control plants were placed back under lights at normal photoperiod for 72 hours in order to allow the psyllids to feed on the leaf (the presence of live feeding psyllids was confirmed). Thereafter, the leaf was snipped off with the organza bag at the base of the petiole and the bag was discarded. Control plants were treated similarly.

Gene Expression Level in LSO Infected Tomatoes

100 tomato seeds were planted. All the plants were transplanted after 10 days post planting. 21 days after planting, 50 plants, were infected with LSO. Leaf samples were taken at 0, 2, 4, 6, 8, days post LSO infection. RNA extraction, cDNA synthesis and qPCR analysis were performed on all samples to measure CalS expression levels.

TABLE 4
Primers used for qPCR analysis

| | |
|---|---|
| CalS_qPCR_F | TCTCAGAAGACGAAGCGTGA/SEQ ID NO: 23 |
| CalS_qPCR_R | CGGAGAGCCTCATTGTCCT/SEQ ID NO: 24 |
| Actin_qPCR_F | TTGCTGACCGTATGAGCAAG/SEQ ID NO: 25 |
| Actin_qPCR R | GGACAATGGATGGACCAGAC/SEQ ID NO: 26 |

Treatment of Tomato Plants with Peptide-dsRNA Complexes

Two weeks old Tiny Tim tomato plants were transferred to bigger pots, 10 days prior to treatment. Four days after psyllid infection, treated leaves were sprayed at two consecutive days with 1% Eco oil spray (EOS) (ADAMA SK EnSpray 99) oil at 10 PSI using a hair brush sprayer. One and a half hours later, oil was washed from the leaves with water and leaves were dried. Then, about 50 μl from the relevant formulation, (final dsRNA concentration 100 μg/ul, molar ratio 8400) was smeared on the selected leaves (2 treated leaves per plant). The different treatment groups are described in Table 5 below:

To prepare peptide-dsRNA complexes, 5 mM peptide solution (produced by centrifuging the peptide vial at maximum speed for 2 min then dissolving 100 mg peptide vial in 2.5 ml UP water in 1 ml aliquots) was added to dsRNA solution while vortexing in sodium phosphate buffer pH 6.8 to a final molar ratio of 8800. Complexes were then incubated at room temperature (RT) for 15 min. 1 ml aliquots were prepared and store at −20° C.

Formation of complexes was verified using a retardation assay. A complex solution containing 500 ng of dsRNA was run on 1% agarose gel for 45 min at 80V. When dsRNA is completely bound by the peptide, it does not migrate on the gel.

Preparation of Cell Wall Degrading Enzymes (CWDE) as Described Herein.

When cell wall degrading enzymes were used (SIGMA, cat. # D9515), they were dissolved in 30 mM sodium phosphate buffer pH 6.8 to a final concentration of 1 mg/ml and allowed to stand at RT for 30 min, to let insolubilized material sediment. CWDE was added just before application on leaves. CWDE supernatant was added to the peptide-dsRNA complexes to a final concentration of 0.1 mg/ml just before smearing on the leaf.

TABLE 5
Treatment Outline

| | A | B | C | D | E |
|---|---|---|---|---|---|
| LSO | + | + | + | + | + |
| Buffer | + | + | + | − | − |
| Peptide | − | + | + | + | − |
| dsCalS | − | − | + | + | − |
| dsRandom | − | + | − | − | − |
| CWDE | − | + | + | − | − |

Plants were kept under red light and 16:8 D:L cycle at 21 C. Disease progression is monitored using DSI scoring system.

DSI Measurements

The following parameters were examined in relevant plants up to 42 days post treatment:

### 1. Height

2. Leaf stiffness
3. Number of flowers
4. Water uptake
5. photosynthesis

Each parameter is scored from 1-5 and an average DSI score is given blindly to each plant, where DSI of 5 is for a plant showing the worst disease symptoms.

Results

CalS Expression Level in LSO Infected Tomatoes

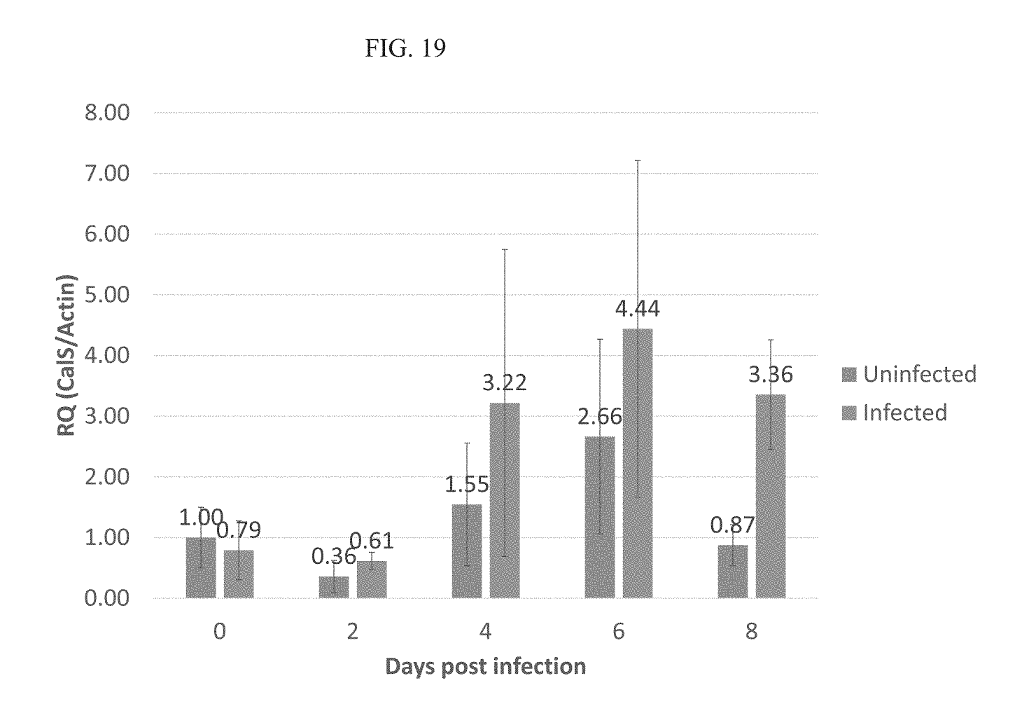

Callose synthase expression (CalS) GenBank Accession Number LOC101249601 is increased in tomato plants in response to infection with LSO. To test CalS expression level in tomatoes, tomato plants were infected with LSO and CalS expression was determined in sampled leaves using qPCR analysis (FIG. 19).

Elevated expression levels of Cals were seen at 4 and 6 days post infection. Therefore, 4 days post infection was elected as the time point for dsRNA treatment.

Effect of CWDE on the Symptom Improvement of LSO Infected Tomatoes

In order to prove the benefit of CWDE in the delivery of dsRNA into the plants and into the target cells, tomato plants were treated with peptide-dsRNA complexes either with or without CWDE. Then, the effect was evaluated using the DSI scoring compared to non-treated plants or plants treated with irrelevant dsRNA sequence (B2) over a period of 42 days.

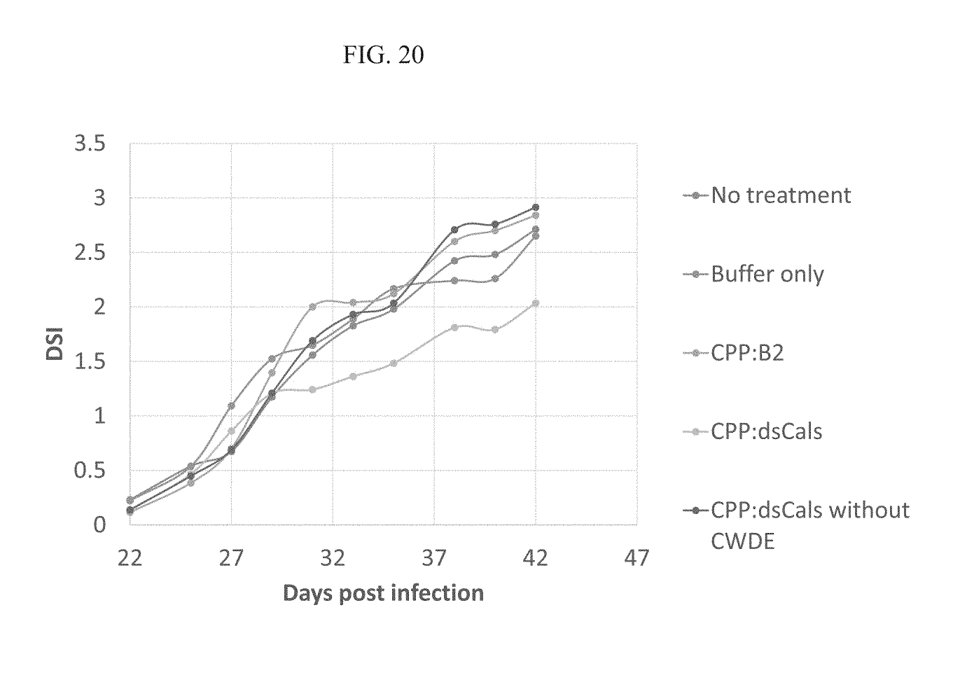

As can be seen from FIG. 20 the DSI of plants treated with peptide, dsRNA and CWDE (in yellow) is lower than the DSI of all other groups, including the group with the dsRNA and peptide but no CWDE (dark blue bar). This implies that only in the presence of CWDE, improvement of phenotype can be seen in the tomato disease model.

Visual representation of the DSI values can be seen in FIG. 22, which represents the plants at 42 days post infection. On the left picture, a representative plant from each group can be seen, where the middle plants treated with peptide, CalS dsRNA and CWDE show less disease symptoms than any other group. This is substantiated when comparing the plant from group D (without CWDE) with group C (with CWDE).

1. A method of delivering a polynucleotide to a plant cell comprising contacting the plant cell with said polynucleotide and at least one cell wall degrading enzyme, and at least one of a nucleic acid condensing agent, a transfection reagent, a surfactant, and a cuticle penetrating agent.

2. (canceled)

3. The method of claim 1, wherein said polynucleotide is a dsRNA.

4. (canceled)

5. The method of claim 3, wherein said dsRNA comprises a nucleotide sequence complementary to a sequence of an mRNA selected from the group consisting of Citrus sinensis magnesium-chelatase subunit ChlI, chloroplastic mRNA (SEQ ID NO: 9) Tomato GPT (tomato Glucose phosphate transporter mRNA (SEQ ID NO: 8), Citrus AGPase (citrus glucose-1-phosphate adenylyltransferase large subunit) mRNA (SEQ ID NO: 7) and Citrus CalS Solanum lycopersicum callose synthase mRNA (SEQ ID NO: 6).

6. The method of claim 1, wherein said cell wall degrading enzyme is selected from the group consisting of cellulases, hemicellulases, lignin-modifying enzymes, cinnamoyl ester hydrolases and pectin-degrading enzymes.

7. The method of claim 1, wherein said at least one cell wall degrading enzyme comprises a combination of cellulases, xylases and laminarinases.

8. The method of claim 1, wherein said nucleic acid condensing agent is selected from the group consisting of protamine, spermidine3+, spermine4+, hexamine cobalt, polycationic peptides such as polylysine and polyarginine, histones H1 and H5 and polymers such as PEG, polyaspartate and polyglutamate.

9. The method of claim 1, wherein said transfection reagent is selected from the group consisting of cationic and polycationic polymers, particles and nanoparticles, and cationic and polycationic lipids.

10. The method of claim 1, wherein said surfactant is selected from the group consisting of an anionic surfactants, cationic surfactants, amphoteric surfactants and non-ionic surfactants.

11. The method of claim 1, wherein said cuticle penetrating agent is selected from the group consisting of an oil, an abrasive, a fatty acid, a wax, a soap and a grease.

12. (canceled)

13. The method of claim 1, wherein said contacting is effected via spraying, dusting, aerosol application or particle bombardment, the method comprising:
contacting a plant or organ thereof comprising the plant cell with said surfactant or cuticle penetrating agent or both, and
subsequently contacting said plant or organ thereof with said polynucleotide and said cell wall degrading enzyme and said at least one of said nucleic acid, said condensing agent, said transfection reagent and said surfactant,
thereby delivering said polynucleotide to said plant cell.

14. The method of claim 1, wherein said contacting is effected via injection, the method comprising injecting a plant or organ thereof comprising the plant cell with said polynucleotide and said cell wall degrading enzyme and at least one of a said nucleic acid condensing agent, said transfection reagent and said surfactant,
thereby delivering said polynucleotide to said plant cell.

15. The method of claim 1, wherein said contacting is effected via irrigation, the method comprising contacting said a plant or organ thereof comprising the plant cell with said polynucleotide and said cell wall degrading enzyme and at least one of a said nucleic acid condensing agent, said transfection reagent and said surfactant, thereby delivering said polynucleotide to said plant cell.

16. The method of claim 1, wherein said plant cell comprises a cell wall.

17. (canceled)

18. A method of expressing a nucleic acid sequence in a plant cell, the method comprising delivering a polynucleotide to cells of said plant according to the method of claim 1, wherein said polynucleotide comprises a nucleic acid construct comprising said nucleic acid sequence transcriptionally connected to a plant expressible promoter.

19. A method of increasing vigor, yield and/or tolerance of a plant to biotic and abiotic stress, the method comprising:
delivering a polynucleotide to cells of said plant according to the method of claim 1, wherein expression of said polynucleotide in said plant increases vigor, yield and/or tolerance of a plant to biotic and abiotic stress of said plant.

20. A method of delivering an agrochemical molecule to a host organism comprising: delivering the agrochemical molecule to a plant comprising:
(a) contacting the plant cell with the agrochemical molecule and a cell wall degrading enzyme and at least one of a nucleic acid condensing agent, a transfection reagent, a surfactant, and a cuticle penetrating agent, thereby delivering the agrochemical molecule to the plant, and
(b) contacting said host organism with said plant,
wherein said host organism ingests cells, tissue or cell contents of said plant.

21. A composition of matter comprising a polynucleotide, a cell wall degrading enzyme and at least one of a nucleic acid condensing agent, a transfection reagent, a surfactant, and a cuticle penetrating agent.

22. The composition of claim 21, wherein said polynucleotide is an RNA or DNA.

23. The composition of claim 22, wherein said polynucleotide is a dsRNA.

24. The composition of claim 23, wherein said dsRNA is selected from the group consisting of siRNA, shRNA and miRNA.

25. The composition of claim 24, wherein said dsRNA comprises a nucleotide sequence complementary to sequence selected from the group consisting of Citrus sinensis magnesium-chelatase subunit ChlI, chloroplastic mRNA (SEQ ID NO: 9) Tomato GPT (tomato Glucose phosphate transporter mRNA (SEQ ID NO: 8), Citrus AGPase (citrus glucose-1-phosphate adenylyltransferase large subunit) mRNA (SEQ ID NO: 7) and Citrus CalS Solanum lycopersicum callose synthase mRNA (SEQ ID NO: 6).

26. The composition of claim 21, wherein said cell wall degrading enzyme is selected from the group consisting of cellulases, hemicellulases, lignin-modifying enzymes, cinnamoyl ester hydrolases and pectin-degrading enzymes.

27. The composition of claim 21, wherein said nucleic acid condensing agent is selected from the group consisting of protamine, spermidine3+, spermine4+, hexamine cobalt, polycationic peptides such as polylysine and polyarginine, histones H1 and H5 and polymers such as PEG, polyaspartate and polyglutamate.

28. The composition of claim 21, wherein said transfection reagent is selected from the group consisting of cationic and polycationic polymers, particles and nanoparticles, and cationic and polycationic lipids.

29. The composition of claim 21, wherein said surfactant is selected from the group consisting of anionic surfactants, cationic surfactants, amphoteric surfactants and non-ionic surfactants.

30. The composition of claim 21, wherein said cuticle penetrating agent is selected from the group consisting of an oil, an abrasive, a fatty acid, a wax, a soap and a grease.

31. (canceled)

32. The composition of claim 21, formulated for spraying or topical administration, comprising said polynucleotide, said cell wall degrading enzyme and at least one of a nucleic acid condensing agent, a transfection reagent, a surfactant, and a cuticle penetrating agent.

33. The composition of claim 21, formulated for irrigation, comprising said polynucleotide, said cell wall degrading enzyme and at least one of a nucleic acid condensing agent, a transfection reagent, a surfactant, and a cuticle penetrating agent.

34. The composition of claim 21, further comprising an agrochemical molecule.

35. The composition of claim 34, wherein said agrochemical molecule is selected from the group consisting of fertilizers, pesticides, fungicides and antibiotics.
